(19) United States
(12) Patent Application Publication
(10) Pub. No.: US 2026/0131029 A1
(43) Pub. Date: May 14, 2026

(54) ENGINEERED NUCLEIC ACID REGULATORY ELEMENTS AND METHODS AND USES THEREOF

(71) Applicant: REGENXBIO INC., Rockville, MD (US)

(72) Inventors: Randolph Qian, Rockville, MD (US); Devin McDougald, Boston, MA (US)

(21) Appl. No.: 19/119,083

(22) PCT Filed: Oct. 11, 2023
(86) PCT No.: PCT/US2023/076622

§ 371 Date: Apr. 7, 2025

Related U.S. Application Data
(60) Provisional application No. 63/415,251, filed on Oct. 11, 2022.

(51) Int. Cl. A61K 48/00; C07K 14/47; C12N 15/864

(52) U.S. Cl. CPC A61K 48/0058; C07K 14/4708; C07K 14/4716; C12N 15/8645; C12N 2750/14143; C12N 2750/14145; C12N 2830/008; C12N 2830/34

(57) ABSTRACT

The present invention relates to nucleic acid expression cassettes that are engineered to enhance gene expression. Vectors and methods employing the expression cassettes containing novel chimeric regulatory elements are provided. The invention is particularly useful for delivery of transgenes to target cells and confers desirable properties for muscle¬gene therapy. Moreover, the invention relates to gene therapy methods of delivery of therapeutics for treating various disorders.

### REFERENCE TO SEQUENCE LISTING

The Sequence Listing submitted Oct. 11, 2023 as an xml file named 38013_0032P1_SEQLIST.xml, created on Oct. 11, 2023, and having a size of 105,583 bytes is hereby incorporated by reference pursuant to 37 C.F.R. §§ 1.831-1.835.

### 1. FIELD OF THE INVENTION

The present invention relates to nucleic acid regulatory elements engineered to enhance gene expression, methods of employing the regulatory elements and uses thereof. Use of the engineered regulatory elements upstream of a transgene delivered to target cells confers desirable properties, and in some cases confers desirable properties for gene therapy. In particular, the invention provides nucleic acid regulatory elements operably linked to a heterologous gene (transgene) inserted into an expression cassette, such that the regulatory elements drive expression of the transgene in specific cells. As such, the invention also provides a method to target tissues, in particular, expression cassettes comprising the engineered regulatory elements improve expression of the transgene in muscle, as well as deliver therapeutics systemically for the treatment of various disorders.

### 2. BACKGROUND

The use of regulatory elements to drive gene expression is highly complex. Both naturally occurring and synthetic regulatory elements, such as enhancers and promoters, have been reported in the art. It is not known whether multiple elements engineered for heterologous gene expression will produce various aberrant, unstable and/or competing transcripts in a given tissue environment.

Gene expression vectors that are highly productive and stable may be suitable for gene therapy. Transgenes delivered with AAV or other viral vectors aim to provide long-term gene expression and thus may boost systemic expression levels or serum half-life of a biotherapeutic transgene. As such, improved gene expression systems for gene therapy would greatly benefit patients compared to direct injection of a biologic drug, such as in enzyme replacement therapy. Although AAV capsid proteins that carry genome DNA can confer a particular tissue tropism to deliver DNA into target cells, it is desirable to express greater amounts of the gene of interest in liver, due to its low immunogenicity (Pastore, et al. Human Gene Therapy Vol. 10(11):1773-81, July 1999).

Thus, expression of a biotherapeutic in muscle well as systemic delivery would be desirable. Muscle is frequently targeted by gene therapies; however, muscle diseases vary widely in terms of pathological etiology and the specific tissues affected. Consequently, expanding the toolbox of available muscle promoters with unique properties is desirable to improve the efficacy of the current generation of muscle-directed gene therapies. Notably, as muscle-targeted gene therapies typically utilize high AAV doses, more potent muscle promoters with low off-target activities are extremely valuable as means of reducing clinically effective doses. There remains a need for tissue-targeted gene expression and vectors that are highly productive in skeletal muscle.

### 3. SUMMARY OF THE INVENTION

Provided are recombinant expression cassettes comprising a composite nucleic acid regulatory element for enhancing or controlling gene expression in the liver and in skeletal muscle, or in skeletal muscle or in cardiac muscle. In embodiments, the liver and/or heart tissue is detargeted. The regulatory element is a composite comprising at least one enhancer and at least one promoter, operably linked to a transgene.

Provided are recombinant expression cassettes comprising a composite nucleic acid regulatory element which comprises one or more promoters arranged in tandem, including in embodiments, comprising two or more promoters (that is, a composite promoter), where the composite promoters are a muscle-specific promoter, including promoters in Table 1. In embodiments, the composite nucleic acid regulatory element comprises a CK promoter. In embodiments, the composite nucleic acid regulatory element comprises an ACTA core promoter (any one of the nucleotide sequences of SEQ ID Nos: 10-13), as in Table 1.

The composite nucleic acid regulatory element comprise one or more enhancer elements, including, in embodiments, two or more enhancer elements. At least one of the enhancer elements may also be muscle specific. In embodiments, the composite nucleic acid regulatory element comprises one or two muscle specific cis regulatory elements (CREs) or Mus CREs. In embodiments the Mus CRE is Mus022 (SEQ ID NO: 10) or may be Mus007 (SEQ ID NO: 11), Mus011 (SEQ ID NO: 12), or Mus035 (SEQ ID NO: 13) (see Table 1). In embodiments, the composite nucleic acid regulatory element comprises one or two muscle specific regulatory elements or enhancers comprising or consisting of a nucleotide sequence of SEQ ID NO: 4, 5, 6, or 7.

In embodiments, the composite nucleic acid regulatory element comprises or consists of a Mus CRE and a CK promoter, for example with the Mus CRE 5′ to the CK promoter. In embodiments, the composite regulatory elements are any one of the nucleotide sequences comprising or consisting of SEQ ID NO: 1, SEQ ID NO: 9, SEQ ID NO: 68, SEQ ID NO: 69 or SEQ ID Nos: 27-54). In embodiments, the composite nucleic acid regulatory element comprises or consists of any one of the nucleotide sequences of SEQ ID NO: 1, 9, 27-54, 68 or 69.

In some embodiments, provided are expression cassettes comprising the composite nucleic acid regulatory element, which is operably linked to a transgene. The transgene may be any one of the genes or nucleic acids encoding the therapeutic proteins listed in, but not limited to, Tables 4A, 4B and 4C. In certain embodiments, the transgene encodes a therapeutic antibody, either having full length heavy and light chains or an antigen binding fragment, such as a Fab fragment or an scFv. In embodiments, the expression cassette is flanked by AAV ITR sequences and may be within a cis plasmid construct for AAV particle production or an artificial genome within an AAV capsid.

In some embodiments, provided are vectors comprising a transgene operably linked to a composite nucleic acid regulatory element comprising or consisting of a nucleic acid sequence which has a muscle CRE (for example, see Table 1) 5′ of a muscle-specific promoter or two promoters in tandem. In embodiments, the muscle specific promoter is a CK promoter, an mSvn100 promoter, an SPc5.12 promoter, or variant thereof, such as SPc5v2 (See WO 2023/17(053), or an ACTA core promoter. In embodiments, the composite nucleic acid regulatory element is Mus022.CK, including an element with a nucleotide sequence comprising or consisting of SEQ ID NO: 9, wherein the transgene is expressed in skeletal muscle. In certain embodiments, the transgene has reduced expression in cardiac muscle or heart tissue when compared to expression in skeletal muscle. In certain embodiments, the transgene has increased expression in cardiac muscle or heart tissue when compared to expression in skeletal muscle. In other embodiments, the transgene has reduced expression in liver when compared to expression in muscle, including skeletal muscle.

Provided are methods for enhancing expression of a transgene, comprising delivery of viral vectors comprising nucleic acid constructs comprising or consisting of the recombinant expression cassettes provided herein. Also provided are methods for enhancing expression of a transgene, comprising delivery of viral vectors comprising nucleic acid constructs having the following components: (1) AAV inverted terminal repeats (ITRs) that flank the expression cassette; (2) control elements, which include a) one or more regulatory elements, including embodiments where the control element comprises or consists of at least one or more of the enhancers of any one of SEQ ID NOs: 10-13, in combination with one or more promoters, b) a poly A signal, and c) optionally an intron; and (3) transgene providing (e.g., coding for) one or more RNA or protein products of interest.

Provided are methods of using nucleic acid constructs comprising or consisting of the recombinant expression cassettes provided herein. Also provided are methods of using nucleic acid constructs having the following components: (1) AAV inverted terminal repeats (ITRs) that flank the expression cassette; (2) control elements, which include a) one or more regulatory elements comprising at least one or more of the enhancers of any one of SEQ ID NOs: 10-13, in combination with a CK promoter, b) a poly A signal, and c) optionally an intron; and (3) a transgene providing (e.g., coding for) one or more RNA or protein products of interest, such as those in Tables 4A, 4B, and 4C. Also provided are methods using nucleic acid constructs having the following components: (1) AAV inverted terminal repeats (ITRs) that flank the expression cassette; (2) control elements, which include a) one or more regulatory elements comprising a Mus022 enhancer (SEQ ID NO: 10), in combination with a CK promoter (SEQ ID NO: 8), b) a poly A signal, and c) optionally an intron; and (3) a transgene providing (e.g., coding for) one or more RNA or protein products of interest, such as those in Tables 4A, 4B, and 4C.

Also provided are methods using nucleic acid constructs having the following components: (1) AAV inverted terminal repeats (ITRs) that flank the expression cassette; (2) control elements, which include a) one or more regulatory elements comprising one or more of the promoters comprising or consisting of any one of SEQ ID NO:s 14-26, alone or in combination with one or more enhancers, b) a poly A signal, and c) optionally an intron; (3) transgene providing (e.g., coding for) one or more RNA or protein products of interest. Also provided are methods using nucleic acid constructs having the following components: (1) AAV inverted terminal repeats (ITRs) that flank the expression cassette; (2) control elements, which include a) one or more regulatory elements at least including one or more of the ACTA core promoters of any one of SEQ ID NOs: 14-26, alone or in combination with one or more enhancers of any one of SEQ ID NOs: 4-7, b) a poly A signal, and c) optionally an intron; (3) transgene providing (e.g., coding for) one or more RNA or protein products of interest, such as those in Tables 4A, 4B, and 4C.

In some embodiments, provided are viral vectors incorporating the recombinant expression cassettes described herein, including recombinant AAVs (rAAVs).

In another aspect, methods of treatment by delivery of rAAVs comprising the nucleic acid expression cassettes described herein are also provided. Provided are methods for treating a disease or disorder, including but not limited to those outlined in Tables 4A and 4B, in a subject in need thereof comprising administration of recombinant AAV particles comprising an expression cassette comprising composite nucleic acid regulatory sequences of any one of SEQ ID NO: 1, SEQ ID NO: 9, SEQ ID NO: 68, SEQ ID NO: 69 or SEQ ID NOs: 27-54.

Also provided are methods of producing recombinant AAV vectors comprising 1) culturing a host cell comprising a) an artificial genome flanked by AAV ITRs and comprising an expression cassette comprising any one of the composite regulatory elements in Table 1 such as SEQ ID NO: 9, or SEQ ID NOs: 27-54, or SEQ ID NOs: 68-69, operably linked to a transgene and b) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans; and c) sufficient adenovirus helper functions to permit replication and packaging of the artificial genome by the AAV capsid protein; and 2) recovering recombinant AAV encapsidating the artificial genome from the cell culture. Host cells for production of recombinant AAV comprising an artificial genome comprising an expression cassette as described herein are also provided.

The invention is illustrated by way of examples infra describing the construction and function of gene cassettes engineered with composite regulatory elements designed on the basis of several liver-specific enhancers and promoters, in tandem with muscle specific enhancers and promoters, whereas the downstream elements are modified at their translation start sites.

### 3.1 EMBODIMENTS

Embodiment 1. A recombinant expression cassette comprising a composite nucleic acid regulatory element comprising a) Muscle cis regulatory element (CRE), and b) a CK promoter, a Spc5-12 promoter, or variant thereof, or an ACTA core promoter, operably linked to a transgene.

Embodiment 2. The recombinant expression cassette of embodiment 1, wherein the muscle CRE is Mus022 (SEQ ID NO: 10), Mus077 (SEQ ID NO: 11), Mus011 (SEQ ID NO: 12) or Mus035 (SEQ ID NO: 13).

Embodiment 3. The recombinant expression cassette of embodiment 2, wherein the muscle CRE is Mus022 (SEQ ID NO: 10).

Embodiment 4. The recombinant expression cassette of any one of embodiments 1 to 3, wherein the composite nucleic acid regulatory element is Mus022.CK (SEQ ID NO: 9).

Embodiment 5. The recombinant expression cassette of embodiment 1, wherein the muscle CRE is eMCK (SEQ ID NO: 4), seMCK (SEQ ID NO: 5), mSYN100E (SEQ ID NO: 6) or SPc5v2 (SEQ ID NO: 7).

Embodiment 6. The recombinant expression cassette of any one of embodiments 1 to 3 or 5, wherein the ACTA promoter is mmACTA1 (SEQ ID NO: 14), mmACTA2 (SEQ ID NO: 15), mmACTA3 (SEQ ID NO: 16), mmACTA4 (SEQ ID NO: 17), huACTA1 (SEQ ID NO: 18), huACTA2 (SEQ ID NO: 19), huACTA3 (SEQ ID NO: 20), mmACTA2-shortUTR (SEQ ID NO: 21), mmACTA2-midUTR (SEQ ID NO: 22), huACTA2-shortUTR (SEQ ID NO: 23), huACTA2-midUTR (SEQ ID NO: 24), mmACTA3-shortUTR (SEQ ID NO: 25) or huACTA3-midUTR (SEQ ID NO: 26).

Embodiment 7. A recombinant expression cassette comprising a composite nucleic acid regulatory element comprising or consisting of a nucleic acid sequence having a) 99%, 95%, 90%, 85% or 80% sequence identity with any one of SEQ ID NOs: 9, 27-54, 68 and 69 and/or having b) 1 to 10 nucleotide substitutions compared to any one of SEQ ID NOs: 9, 27-54, 68 and 69, wherein the composite nucleic acid regulatory element retains biological activity of any one of SEQ ID NOs: 9, 27-54, 68 and 69.

Embodiment 8. The recombinant expression cassette of embodiment 7, wherein the composite nucleic acid regulatory element comprises any one of the nucleotide sequences of SEQ ID NO: 9, 27-54, 68, or 69.

Embodiment 9. The recombinant expression cassette of embodiment 8, wherein the composite nucleic acid regulatory element consists of any one of the nucleotide sequences of SEQ ID NO: 9, 27-54, 68, or 69

Embodiment 10. The recombinant expression cassette of any one of embodiments 1 to 9, further comprising an intron sequence between the composite nucleic acid regulatory element and the transgene.

Embodiment 11. The recombinant expression cassette of any one of embodiments 1 to 10, wherein the transgene is a gene or nucleic acid encoding a therapeutic listed in Tables 4A-4C.

Embodiment 12. The recombinant expression cassette of embodiment 11, wherein the transgene is a muscle-derived protein.

Embodiment 13. The recombinant expression cassette of embodiment 11, wherein the transgene is a minidystrophin gene or a microdystrophin gene.

Embodiment 14. The recombinant expression cassette of embodiment 13, wherein microdystrophin is Dys1 (SEQ ID NO: 73), Dys3 (SEQ ID NO: 74), Dys5 (SEQ ID NO: 75), human MD1 (R4-R23/ACT) (SEQ ID NO: 76), Human microdystrophin (SEQ ID NO: 77), Dys3978 (SEQ ID NO: 78), Human MD3 (SEQ ID NO: 79) or Human MD4 (SEQ ID NO: 80).

Embodiment 15. The recombinant expression cassette of any one of embodiments 1 to 10, wherein the transgene is a gene or nucleic acid encoding a therapeutic antibody listed in Table 4B, or antigen binding fragment thereof.

Embodiment 16. A vector comprising the recombinant expression cassette of any one of embodiments 1 to 15.

Embodiment 17. The vector of embodiment 16, further comprising AAV ITRs flanking the expression cassette.

Embodiment 18. The vector of embodiment 16 or embodiment 17 wherein the recombinant expression cassette is suitable for packaging in an ssAAV or scAAV vector.

Embodiment 19. An rAAV particle comprising the vector of any one of embodiments 16 to 18, and a capsid protein from an AAV capsid serotype selected from AAV 1. AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37. AAV.hu32, AAV.Anc80, AAV.Anc80L65, AAV.7m8, AAV.PHP.B, AAV2.5, AAV2tYF. AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

Embodiment 20. A method for enhancing expression of a transgene in a subject, said method comprising delivering a viral vector comprising a recombinant expression cassette comprising a composite nucleic acid regulatory element comprising in a 5′ to 3′ arrangement a) a Mus022 sequence; b) at least one muscle-specific promoter; c) a transgene; and d) a polyadenylation signal sequence.

Embodiment 21. The method of embodiment 20 wherein the muscle specific promoter is CK promoter (SEQ ID NO: 8).

Embodiment 22. The method of embodiment 20 or embodiment 21, wherein the viral vector is administered intravenously or intramuscularly.

Embodiment 23. The method of any one of embodiments 20 to 22, wherein transgene expression is enhanced in circulation or systemically.

Embodiment 24. The method of any one of embodiments 20 to 23, wherein transgene expression is enhanced in liver or skeletal muscle.

Embodiment 25. A method of treatment comprising delivering a rAAV comprising the recombinant expression cassette of any one of embodiments 1-15, or the vector of any one of embodiments 16 to 18, or delivering the rAAV of embodiment 19.

### 4. BRIEF DESCRIPTION OF THE FIGURES

FIG. 1 Depiction of an AAV genome cassette, arranged 5′ to 3′: 5′-ITR, muscle CRE (Mus CRE), CK promoter, optional intron, gene of interest, poly adenylation (polyA) sequence, 5′-ITR.

FIGS. 2A-2D: FIG. 2A: Depiction of a reporter gene cassette, arranged 5′ to 3′: 5′-ITR, muscle CRE (Mus CRE), CK promoter, intron, eGFP transgene, barcode sequence, poly adenylation (polyA) sequence, 3′-ITR. FIG. 2B: Representative micrographs for each CRE candidate cassette as transfected into differentiated C2C12 cells and exhibiting eGFP expression (fluorescence). FIG. 2C: Data graph depicting fold-change of CRE activity for each plasmid normalized to a control plasmid (CK promoter with no upstream CRE). FIG. 2D depicts relative promoter activity, e.g. transgene RNA transcripts relative abundance over DNA relative abundance in C2C12 cells.

FIG. 3. depicts normalized promoter activity in mouse tissues: liver, tibialis anterior (TA), gastrocnemius (GAS), and heart.

FIGS. 4A-D. FIG. 4A: depicts microdystrophin transcript copies per copies of TBP in mouse tissues: gastrocnemius (GAS), tibialis anterior (TA), liver and heart. FIG. 4B: Representative DNA biodistribution of CK7 compared to Mus022.CK vector. FIG. 4C: Representative immunofluorescence depicting transgene expression in heart tissues. FIG. 4D: Representative immunofluorescence depicting transgene expression in GAS tissues.

FIG. 5. Representative AAV genome cassettes depicting various composite nucleic acid regulatory element (enhancer/promoter) combinations, as in Table 1.

FIG. 6. Representative AAV genome cassettes depicting various composite nucleic acid regulatory element (enhancer/promoter) combinations, as in Table 1, and their approximate promoter length in base pairs (bp).

FIG. 7. Representative AAV genome cassettes depicting various composite nucleic acid regulatory element (enhancer/promoter) combinations, as in Table 1.

FIGS. 8A-C. Representative bar graphs depicting promoter activity of the eMCK.mmACTA2shortUTR (EMP1), SPC5v2.mmACTA2shortUTR (EMP2) and eMCK.SPC5v2.mmACTA2shortUTR (EMP3) promoters compared to CK7 and spc5-12 (FIG. 8A), or normalized data compared to CK7 (FIGS. 8B and 8C).

FIGS. 9A-F. Representative bar graphs depicting promoter activity in mouse tissues determined as the relative abundance of RNA versus DNA gastrocnemius (GAS; FIG. 9A), quadricep (Quad; FIG. 9B), tibialis anterior (TA; FIG. 9C), diaphragm (DIA;FIG. 9D), heart (FIG. 9E), and liver (FIG. 9F) for the promoters listed in Table 6.

FIGS. 10A-F. Representative bar graphs depicting promoter activity in monkey tissues determined as the relative abundance of RNA versus DNA in gastrocnemius (GAS; FIG. 10A), quadricep (Quad; FIG. 10B), tibialis anterior (TA; FIG. 10C), diaphragm (DIA;FIG. 10D), heart (FIG. 10E), and liver (FIG. 10F) for the promoters listed in Table 6.

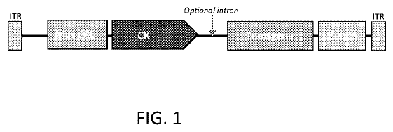

### 5. DETAILED DESCRIPTION

Provided are unique combinations of promoter and enhancer sequences in expression cassettes suitable for improvement of transgene expression while maintaining or conferring tissue specificity. Provided are vectors, such as viral vectors, incorporating the recombinant expression cassettes described herein, including rAAVs, for use in therapy, and methods and host cells for producing same. The novel regulatory element nucleic acids were generated to improve and target transgene expression. Ultimately, these designs may improve the therapeutic efficacy of gene transfer by providing more robust levels of transgene expression, improved stability/persistence, and reduced expression to undesirable tissues which may enable lower dosing. Provided are composite regulatory elements which promote expression in skeletal muscle while having minimal expression in heart or cardiac muscle tissue or liver tissue or, in embodiments, increased expression in cardiac muscle tissue.

### 5.1. Definitions

The term “regulatory element” or “nucleic acid regulatory element” are non-coding nucleic acid sequences that control the transcription of neighboring genes. Cis regulatory elements typically regulate gene transcription by binding to transcription factors. This includes “composite nucleic acid regulatory elements” comprising more than one enhancer or promoter elements as described herein.

The term “expression cassette” or “nucleic acid expression cassette” refers to nucleic acid molecules that include one or more transcriptional control elements including, but not limited to promoters, enhancers and/or regulatory elements, introns and polyadenylation sequences. The enhancers and promoters typically function to direct (trans)gene expression in one or more desired cell types, tissues or organs.

The term “operably linked” and “operably linked to” refers to nucleic acid sequences being linked and typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked and still be functional while not directly contiguous with a downstream promoter and transgene.

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into or modification of the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

The terms “nucleic acids” and “nucleotide sequences” include DNA molecules (e.g., cDNA or genomic DNA). RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

The terms “subject”, “host”, and “patient” are used interchangeably. As used herein, a subject is preferably a mammal such as a non-primate (e.g., cows, pigs, horses, cats, dogs, rats etc.) or a primate (e.g., monkey and human), most preferably a human.

The terms “therapeutic agent” or “biotherapeutic agent” refer to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

The phrase “muscle-specific” or “muscle-directed” refers to nucleic acid elements that have adapted their activity in muscle cells or tissue due to the interaction of such elements with the intracellular environment of the muscle cells. Muscle cells include skeletal muscle as well as cardiac muscle. Secretion of transgene product into the muscle, and/or bloodstream may also be enhanced following various routes of administration, such as intravenous or intramuscular administration, due to intramuscular expression where muscle-specific promoters are present. Various therapeutics benefit from muscle-specific expression of the transgene, or from both muscle-specific and liver-specific expression of the transgene. Muscle production of a biotherapeutic agent (such as produced by the delivered transgene) may provide also provide the host with increased immunotolerance to the agent, as compared to direct injection of an equivalent protein agent to the host.

### 5.2. Regulatory Elements

One aspect relates to nucleic acid regulatory elements that are chimeric with respect to arrangements of elements in tandem in the expression cassette. Regulatory elements, in general, have multiple functions as recognition sites for transcription initiation or regulation, coordination with cell-specific machinery to drive expression upon signaling, and to enhance expression of the downstream gene.

Provided are arrangements of combinations of nucleic acid regulatory elements that promote transgene expression in muscle (including skeletal muscle) tissue. In particular, certain elements are arranged with one or more copies of the individual enhancer and promoter elements arranged in tandem and operably linked to a transgene to promote expression, particularly tissue specific expression. Exemplary nucleotide sequences of the individual promoter and enhancer elements are provided in Table 1. Also provided in Table 1 are exemplary composite nucleic acid regulatory elements comprising promoter and enhancer elements, including one or two promoters and/or one or two enhancer elements. In certain embodiments the downstream promoter is an ACTA core promoter (for example, SEQ ID NO: 14-26) or a CK promoter (SEQ ID NO: 8).

Accordingly, with respect to muscle specific expression, provided are nucleic acid regulatory elements that comprise or consist of promoters and other nucleic acid elements, such as enhancers. In embodiments, the enhancers muscle specific expression, such muscle CREs, including Mus022 (SEQ ID NO: 10), and also including, Mus007 (SEQ ID NO: 11), Mus011 (SEQ ID NO: 12) and Mus035 (SEQ ID NO: 13). These may be present as single copies or with two or more copies (or two different promoters) in tandem.

The recombinant expression cassettes provided herein comprise i) a composite nucleic acid regulatory element comprising a) a muscle specific enhancer region, for example, a Mus CRE, including Mus022 (SEQ ID NO: 10), b) a muscle-specific promoter, including a CK promoter (SEQ ID NO: 8), and c) optionally an intron, and ii) a transgene, to which the composite nucleic acid regulatory element is operably linked, and other regulatory elements, such as polyadenylation signals. In some embodiments, the composite nucleic acid regulatory element comprises mus022.CK (SEQ ID NO: 9) of Table 1. In some embodiments, the composite nucleic acid regulatory element is operably linked to a transgene. The transgene may be any one of the genes or nucleic acids encoding the therapeutic proteins listed in, but not limited to, Tables 4A, 4B and 4C. The transgene may also encode a therapeutic antibody, including a full length antibody or an antigen binding fragment, such as a Fab fragment.

Also provided are nucleic acid regulatory elements which comprise a Muscle CRE (including Mus022 (SEQ ID NO: 10). Mus007 (SEQ ID NO: 11), Mus011 (SEQ ID NO: 12), or Mus035 (SEQ ID NO: 13)) upstream (5′) of a muscle-specific promoter, including CK promoter or any other muscle specific promoter (see Table 1), for example, Spc5-12 which, in embodiments, are operably linked to a transgene.

Provided are composite regulatory elements that enhance gene expression in the skeletal muscle and/or cardiac muscle and which have 99%. 95%, 90%, 85% or 80% sequence identity with SEQ ID NO: 9 (Mus022.CK) and/or comprise 1 to 10 nucleic acid substitutions while retaining biological activity of the composite regulatory element. Also provided are composite regulatory elements that enhance gene expression in the skeletal muscle and/or cardiac muscle and that comprise or consist of SEQ ID NO: 9.

Provided are composite regulatory elements that enhance gene expression in the skeletal muscle and/or cardiac muscle and which have 99%, 95%, 90%, 85% or 80% sequence identity with any one of SEQ ID NOs: 27-54, 68 and 69 (composite enhancer/ACTA core promoters) and/or comprise 1 to 10 nucleic acid substitutions while retaining biological activity of the composite regulatory element. Also provided are composite regulatory elements that enhance gene expression in the skeletal muscle and/or cardiac muscle and that comprise or consist of any one of SEQ ID NOs: 27-54, 69 and 69.

The terms “sequence identity”, “percent sequence identity” or “percent identical” in the context of nucleic acid sequences refers to the residues in the two sequences which are the same when aligned for maximum correspondence. The length of sequence identity comparison may be over the full-length of a gene sequence or component as provided in the sequence listing or claimed, e.g. an enhancer or promoter or composite promoter sequence, or a fragment or portion thereof, for example over a nucleotide sequence encoding a composite enhancer/ACTA core promoter. However, identity among smaller fragments, e.g. of at least about ten nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment or portion thereof, such as for transgene protein products or capsid proteins.

Alignments are performed using any of a variety of publicly or commercially available Multiple Sequence Alignment Programs, such as Clustal W, accessible through web servers on the internet. There are a number of algorithms known in the art which can be used to measure nucleotide sequence identity, including those contained in the programs described herein. Similar programs are available for amino acid sequences, e.g., the Clustal X program (Soding, J. (2005) Bioinformatics 21, 951-960; Thompson J D et al., (1994) Nucleic Acids Res. 22(22): 4673-4680; Larkin, M A et al. (2007) Bioinformatics, 23, 2947-2948). Generally, any of these programs can be used at default settings, although one of skill in the art can alter these settings as needed. Alternatively, one of skill in the art can utilize another algorithm or computer program which provides at least the level of identity or alignment as that provided by the referenced algorithms and programs (Higgins D G et al., (2005) PNAS USA; 102(30): 10411-10412; Raghava and Barton (2006) BMC Bioinformatics, 7:415).

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. In some instances, the homology is over a full-length sequence, or an open reading frame thereof, e.g., a promoter, an anhancer, a cap sequence, a rep sequence, a transgene, or another suitable fragment which is at least 15 nucleotides in length. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. In some instances, the homology is over a full-length sequence, or a protein thereof, e.g., a cap protein, a rep protein, a therapeutic protein (transgene product), or a fragment or portion thereof which is at least 8 amino acids, or at least 15 amino acids in length. Examples of suitable fragments are described herein.

In an aspect of the invention, various regulatory elements and combinations of elements were utilized to design and generate nucleic acid expression cassettes, and are listed in Table 1.

TABLE 1
Engineered Muscle Promoters (EMPs) and EMP Elements

| | SEQ | |
|---|---|---|
| | ID | |
| Name | NO: | Nucleic Acid Sequence |
| CK7 | 1 | ccactacgggtttaggctgcccatgtaaggaggcaaggcctggggacac |
| (modified | | ccgagatgcctggttataattaacccagacatgtggctgcccccccccc |
| MCK enhancer | | ccccaacacctgctgcctctaaaaataaccctgtccctggtggatcggt |
| (eMCK) + CK | | acccgggCCCTGCATGCGAAGATCTTCGAACAAGGCTGTGGGGGACTGA |
| promoter) | | GGGCAGGCTGTAACAGGCTTGGGGGCCAGGGCTTATACGTGCCTGGGAC |
| | | TCCCAAAGTATTACTGTTCCATGTTCCCGGCGAAGGGCCAGCTGTCCCC |
| | | CGCCAGCTAGACTCAGCACTTAGTTTAGGAACCAGTGAGCAAGTCAGCC |
| | | CTTGGGGCAGCCCATACAAGGCCATGGGGCTGGGCAAGCTGCACGCCTG |
| | | GGTCCGGGGTGGGCACGGTGCCCGGGCAACGAGCTGAAAGCTCATCTGC |
| | | TCTCAGGGGCCCCTCCCTGGGGACAGCCCCTCCTGGCTAGTCACACCCT |
| | | GTAGGCTCCTCTATATAACCCAGGGGCACAGGGGCTGCCCTCATTCTAC |
| | | CACCACCTCCACAGCACAGACAGACACTCAGGAGCCAGCCAGCGTCGA |
| mSyn100 | 2 | cggccgtccgccctcggcaccattcctcacgacaccCaaatatggcgac |
| Promoter | | gggtgaggaatggtggggagttatttttagagcggtgaggaatggtggg |
| | | caggcagcaggtgttgggggagttatttttagagcggggagttattttt |
| | | agagcggtgaggaatggtggacaccCaaatatggcgacgggtgaggaat |
| | | ggtgccgtcgccatatttgggtgtcccgtccgccctcggccggggccgc |
| | | attcctgggggccgggcggtgctcccgcccgcctcgataaaaggctccg |
| | | gggccggcggcggcccacgagctacccggaggagcgggaggcgtctctg |
| | | ccagcggtccgacgcgcagtcagcaccaggtaggtgggcaccgcgccgt |
| | | gccgtgcc |
| SPc5.12 | 3 | ggccgtccgccctcggcaccatcctcacgacacccaaatatggcgacgg |
| Promoter | | gtgaggaatggtggggagttatttttagagcggtgaggaaggtgggcag |
| (Li, et al. | | gcagcaggtgttggcgctctaaaaataactcccgggagttatttttaga |
| Nature | | gcggaggaatggtggacacccaaatatggcgacggttcctcacccgtcg |
| Biotechnology | | ccatatttgggtgtccgccctcggccggggccgcattcctgggggccgg |
| 17: 241-245 | | gcggtgctcccgcccgcctcgataaaaggctccggggccggcggcggcc |
| (1999)) | | cacgagctacccggaggagcgggaggcgccaagc |
| eMCK | 4 | ccactacgggtttaggctgcccatgtaaggaggcaaggcctggggacac |
| modified MCK | | ccgagatgcctggttataattaacccagacatgtggctgcccccccccc |
| enhancer | | ccccaacacctgctgcctctaaaaataaccctgtccctggtggatc |
| (eMCK) | | |
| seMCK | 5 | tgcctggttataattaacccagacatgtggctgccccccccccccccaa |
| short modified | | cacctgctgcctctaaaaataaccctgtccctggtggatc |
| MCK enhancer | | |
| (seMCK) | | |
| mSYN100E | 6 | cggccgtccgccctcggcaccattcctcacgacaccCaaatatggcgac |
| Enhancer | | gggtgaggaatggtggggagttatttttagagcggtgaggaatggtggg |
| | | caggcagcaggtgttgggggagttatttttagagcggggagttattttt |
| | | agagcggtgaggaatggtggacaccCaaatatggcgacgggtgaggaat |
| | | ggtgccgtcgccatatttgggtgtcccgtccgccctc |
| SPc5v2 | 7 | GGCCGTCCGCCCTCGGCACCATCCTCACGACACCCAAATATGGCGACGG |
| Promoter | | GTGAGGAATGGTGGGGAGTTATTTTTAGAGCGGTGAGGAAGGTGGGCAG |
| Portion (See | | GCAGCAGGTGTTGGGGGAGTTATTTTTAGAGCGGGGAGTTATTTTTAGA |
| WO | | GCGGAGGAATGGTGGACACCCAAATATGGCGACGGTTCCTCACGGACAC |
| 2023/178053) | | CCAAATATGGCGACGGGCCCT |
| CK Promoter | 8 | ccctgcatgcgaagatcttcgaacaaggctgtgggggactgagggcagg |
| | | ctgtaacaggcttgggggccagggcttatacgtgcctgggactcccaaa |
| | | gtattactgttccatgttcccggcgaagggccagctgtcccccgccagc |
| | | tagactcagcacttagtttaggaaccagtgagcaagtcagcccttgggg |
| | | cagcccatacaaggccatggggctgggcaagctgcacgcctgggtccgg |
| | | ggtgggcacggtgcccgggcaacgagctgaaagctcatctgctctcagg |
| | | ggcccctccctggggacagcccctcctggctagtcacaccctgtaggct |
| | | cctctatataacccaggggcacaggggctgccctcattctaccaccacc |
| | | tccacagcacagacagacactcaggagccagccagcgtcga |
| Mus022.CK | 9 | CTCCCGTTTGACCCAAGCTTCCTGAGCTCTCCTCCCATTCCCCTTTTTG |
| | | GAGTCCTCCTCCTCTCCCAGAACCCAGTAATAAGTGGGCTCCTCCCTGG |
| | | CCTGGACCCCCGTGGTAACCCTATAAGGCGAGGCAGCTGCTGTCTGAGG |
| | | CAGGGAGGGGCTGGTGTGGGAGGCTAAGGGCAGCTGCTAAGTTTAGGGT |
| | | GGCTCCTTCTCTCTTCTTAGAGACAACAGGTGGCTGGGGCCTCAGTGCC |
| | | CAGAAAAGAAAATGTCTTAGAGGTATCGGCATGGGCCTGGAGGAGGGGG |
| | | GACAGGGCAGGGGGAGGCATCTTCCTCAGGACATCGGGTCCTAGAGGGA |
| | | GCGGAGAGGGGTACCCGGGCCCTGCATGCGAAGATCTTCGAACAAGGCT |
| | | GTGGGGGACTGAGGGCAGGCTGTAACAGGCTTGGGGGCCAGGGCTTATA |
| | | CGTGCCTGGGACTCCCAAAGTATTACTGTTCCATGTTCCCGGCGAAGGG |
| | | CCAGCTGTCCCCCGCCAGCTAGACTCAGCACTTAGTTTAGGAACCAGTG |
| | | AGCAAGTCAGCCCTTGGGGCAGCCCATACAAGGCCATGGGGCTGGGCAA |
| | | GCTGCACGCCTGGGTCCGGGGTGGGCACGGTGCCCGGGCAACGAGCTGA |
| | | AAGCTCATCTGCTCTCAGGGGCCCCTCCCTGGGGACAGCCCCTCCTGGC |
| | | TAGTCACACCCTGTAGGCTCCTCTATATAACCCAGGGGCACAGGGGCTG |
| | | CCCTCATTCTACCACCACCTCCACAGCACAGACAGACACTCAGGAGCCA |
| | | GCCAGCGTCGA |
| Mus022 | 10 | ctcccgtttgacccaagcttcctgagctctcctcccattcccctttttg |
| Enhancer | | gagtcctcctcctctcccagaacccagtaataagtgggctcctccctgg |
| | | cctggacccccgtggtaaccctataaggcgaggcagctgctgtctgagg |
| | | cagggaggggctggtgtgggaggctaagggcagctgctaagtttagggt |
| | | ggctccttctctcttcttagagacaacaggtggctggggcctcagtgcc |
| | | cagaaaagaaaatgtcttagaggtatcggcatgggcctggaggaggggg |
| | | gacagggcagggggaggcatcttcctcaggacatcgggtcctagaggga |
| | | gcgg |
| Mus007 | 11 | AACCATTGTGGGCAGCCAGCCTGGCGGGAGAGGCTAGGGCACGAAGCGC |
| Enhancer | | CGCCCAGTTACCATGGTAACACTCCCTCGTCCCAACCCCGTCCCAGTTT |
| | | ACTGGCCCAAGGTAAGGCCGGGAGCCGTACTCCAGGGCGACTGGGAGGA |
| | | TCCACTCCCTGCCGCGGCTCTCCCTCCAGTGGACTGCCTTCCCGCCTGC |
| | | CTGGCGGTGGGAGGGCGCGGCAGAGGGACGTCACATCCGGGCGGGTTGG |
| | | TGAGTTCCGGTATTTCAGGGCGTAGCAGGCGGAAGTAAGGGTGAGAGGA |
| | | GGCTGCAACGCCGAGCGGAGGAGGCAGGAACCGGAGCGCGAGCAGTAGC |
| | | TGGGTGGG |
| Mus011 | 12 | CTCCCCTCTTCCTCTTCTTTTAAAACATCCATAGAGCTGCCCAGAATGA |
| Enhancer | | GTGGAAAGTTTCAGGGTCAGGGAGAACCTCTTCCCTCCCCCACTTTCCA |
| | | TTGTCTAAAATCAGCCTCAGCTGTTGCTAGATATTTTTTTTGCTGGTGA |
| | | TGTCAGTTCATCAAAGCGTCTCACCCTGTGTTCCCCCAGGAGAACAATG |
| | | ATGGGGTGGAGGGAGCTGGAAAGACCATATTAGGAACAAAAGGTGGAGC |
| | | TATGAGCACAGATAAAGACTCAAGTCTGGGGACCTCCTGGTCACTCAGG |
| | | CAGCAGCCCCTTCTTTCTTGCCCCAGTCTCCAGTTCTCCAGTGTTCACA |
| | | GGT |
| Mus035 | 13 | GGCACTGGGGTGTGTCTCCTTAGGACACCCCTGCCCATTACTCTCTCCT |
| Enhancer | | TCTCCAAATGGGGAGCATGGCTGGGGCTCCCTAACCTCCTGCTTGCGAG |
| | | GCCTCTCTCTGGCCTCTGAGAGGGTCAGTGTCCTGCCCCAACCCATGAG |
| | | ATGACAGACTATAATAGCCACAGGATTAACATAGCAGGCATTGTCTTTC |
| | | TCTGACTATAGGGTGGGTATTATGTGTTCATCAACCATCCTAAAAATAC |
| | | CCGGTAAACAGGTGCAGCCCCTGTGGCTCCAGTCCCCTGGGATCTGTTG |
| | | GCTTCTGGCTGGAGATGAAGATTAGGGCAGAGGAGA |
| mmACTA1 | 14 | GAAGGGCAGCAACATTCCTTCGGGGCGGTGTGGGGAGAGCTCCCGGGAC |
| Promoter | | TATATAAAAACCTGTGCAAGGGGACAGGCGGTCACACGGACGTGAAGCC |
| | | TCACTTCCTACCCTCGGCACCCAGGGCCAGAGTCAGAGCAGCACTTAGG |
| | | GTGGAGGTGGGGAGGGTGACCTCGAGACCCAGCAAAGAAAG |
| mmACTA2 | 15 | AGTGCCCGACACCCAAATATGGCTTGGGAAGGGCAGCAACATTCCTTCG |
| Promoter | | GGGCGGTGTGGGGAGAGCTCCCGGGACTATATAAAAACCTGTGCAAGGG |
| | | GACAGGCGGTCACACGGACGTGAAGCCTCACTTCCTACCCTCGGCACCC |
| | | AGGGCCAGAGTCAGAGCAGCACTTAGGGTGGAGGTGGGGAGGGTGACCT |
| | | CGAGACCCAGCAAAGAAAG |
| mmACTA3 | 16 | AGTTGGGAGGGGCAGACAGCTGGGGATACTCTCCATATACGGCCTGGTC |
| Promoter | | CGGTCCTAGCTACCTGGGCCAGGGCCAGTCCTCTCCTTCTTTGGTCAGT |
| | | GCAGGAGACCCGGGCGGGGACCCAGGCTGAGAACCAGCCGAAGGAAGGG |
| | | ACTCTAGTGCCCGACACCCAAATATGGCTTGGGAAGGGCAGCAACATTC |
| | | CTTCGGGGCGGTGTGGGGAGAGCTCCCGGGACTATATAAAAACCTGTGC |
| | | AAGGGGACAGGCGGTCACACGGACGTGAAGCCTCACTTCCTACCCTCGG |
| | | CACCCAGGGCCAGAGTCAGAGCAGCACTTAGGGTGGAGGTGGGGAGGGT |
| | | GACCTCGAGACCCAGCAAAGAAAG |
| mmACTA4 | 17 | AGTTGGGAGGGGCAGACAGCTGGGGATACTCTCCATATACGGCCTGGTC |
| Promoter | | CGGTCCAGTGCCCGACACCCAAATATGGCTTGGGAAGGGCAGCAACATT |
| | | CCTTCGGGGCGGTGTGGGGAGAGCTCCCGGGACTATATAAAAACCTGTG |
| | | CAAGGGGACAGGCGGTCACACGGACGTGAAGCCT |
| huACTA1 | 18 | GAAGGGCAGCGACATTCCTGCGGGGTGGCGCGGAGGGAATGCCCGCGGG |
| Promoter | | CTATATAAAACCTGAGCAGAGGGACAAGCGGCCACCGCAGCGGACAGCG |
| | | CCAAGTGAAGCCTCGCTTCCCCTCCGCGGCGACCAGGGCCCGAGCCGAG |
| | | AGTAGCAGTTGTAGCTACCCGCC |
| huACTA2 | 19 | GTGCCCAACACCCAAATATGGCTCGAGAAGGGCAGCGACATTCCTGCGG |
| Promoter | | GGTGGCGCGGAGGGAATGCCCGCGGGCTATATAAAACCTGAGCAGAGGG |
| | | ACAAGCGGCCACCGCAGCGGACAGCGCCAAGTGAAGCCTCGCTTCCCCT |
| | | CCGCGGCGACCAGGGCCCGAGCCGAGAGTAGCAGTTGTAGCTACCCGCC |
| huACTA3 | 20 | AGGGGCAAACCCGCTAGGGAGACACTCCATATACGGCCCGGCCCGCGTT |
| Promoter | | ACCTGGGACCGGGCCAACCCGCTCCTTCTTTGGTCAACGCAGGGGACCC |
| | | GGGGGGGGCCCAGGCCGCGAACCGGCCGAGGGAGGGGGCTCTAGTGCC |
| | | CAACACCCAAATATGGCTCGAGAAGGGCAGCGACATTCCTGCGGGGTGG |
| | | CGCGGAGGGAATGCCCGCGGGCTATATAAAACCTGAGCAGAGGGACAAG |
| | | CGGCCACCGCAGCGGACAGCGCCAAGTGAAGCCTCGCTTCCCCTCCGCG |
| | | GCGACCAGGGCCCGAGCCGAGAGTAGCAGTTGTAGCTACCCGCC |
| mmACTA2- | 21 | AGTGCCCGACACCCAAATATGGCTTGGGAAGGGCAGCAACATTCCTTCG |
| shortUTR | | GGGCGGTGTGGGGAGAGCTCCCGGGACTATATAAAAACCTGTGCAAGGG |
| Promoter | | GACAGGCGGTCACACGGACGTGAAGCCT |
| mmACTA2- | 22 | AGTGCCCGACACCCAAATATGGCTTGGGAAGGGCAGCAACATTCCTTCG |
| midUTR | | GGGCGGTGTGGGGAGAGCTCCCGGGACTATATAAAAACCTGTGCAAGGG |
| Promoter | | GACAGGCGGTCACACGGACGTGAAGCCTCACTTCCTACCCTCGGCACCC |
| | | AGGGCCAGAGTCAGAGCA |
| huACTA2- | 23 | GTGCCCAACACCCAAATATGGCTCGAGAAGGGCAGCGACATTCCTGCGG |
| shortUTR | | GGTGGCGCGGAGGGAATGCCCGCGGGCTATATAAAACCTGAGCAGAGGG |
| Promoter | | ACAAGCGGCCACCGCAGCGGACAG |
| huACTA2- | 24 | GTGCCCAACACCCAAATATGGCTCGAGAAGGGCAGCGACATTCCTGCGG |
| midUTR | | GGTGGCGCGGAGGGAATGCCCGCGGGCTATATAAAACCTGAGCAGAGGG |
| Promoter | | ACAAGCGGCCACCGCAGCGGACAGCGCCAAGTGAAGCCTCGCTTCCCCT |
| | | CCGCGGCGACCA |
| mmACTA3- | 25 | AGTTGGGAGGGGCAGACAGCTGGGGATACTCTCCATATACGGCCTGGTC |
| shortUTR | | CGGTCCTAGCTACCTGGGCCAGGGCCAGTCCTCTCCTTCTTTGGTCAGT |
| Promoter | | GCAGGAGACCCGGGCGGGGACCCAGGCTGAGAACCAGCCGAAGGAAGGG |
| | | ACTCTAGTGCCCGACACCCAAATATGGCTTGGGAAGGGCAGCAACATTC |
| | | CTTCGGGGCGGTGTGGGGAGAGCTCCCGGGACTATATAAAAACCTGTGC |
| | | AAGGGGACAGGCGGTCACACGGACGTGAAGCCT |
| huACTA3- | 26 | AGGGGCAAACCCGCTAGGGAGACACTCCATATACGGCCCGGCCCGCGTT |
| midUTR | | ACCTGGGACCGGGCCAACCCGCTCCTTCTTTGGTCAACGCAGGGGACCC |
| Promoter | | GGGCGGGGGCCCAGGCCGCGAACCGGCCGAGGGAGGGGGCTCTAGTGCC |
| | | CAACACCCAAATATGGCTCGAGAAGGGCAGCGACATTCCTGCGGGGTGG |
| | | CGCGGAGGGAATGCCCGCGGGCTATATAAAACCTGAGCAGAGGGACAAG |
| | | CGGCCACCGCAGCGGACAGCGCCAAGTGAAGCCTCGCTTCCCCTCCGCG |
| | | GCGACCA |
| eMCK.mmAC | 27 | ccactacgggtttaggctgcccatgtaaggaggcaaggcctggggacac |
| TA1 | | ccgagatgcctggttataattaacccagacatgtggctgcccccccccc |
| | | ccccaacacctgctgcctctaaaaataaccctgtccctggtggatcggt |
| | | acccgggGAAGGGCAGCAACATTCCTTCGGGGCGGTGTGGGGAGAGCTC |
| | | CCGGGACTATATAAAAACCTGTGCAAGGGGACAGGCGGTCACACGGACG |
| | | TGAAGCCTCACTTCCTACCCTCGGCACCCAGGGCCAGAGTCAGAGCAGC |
| | | ACTTAGGGTGGAGGTGGGGAGGGTGACCTCGAGACCCAGCAAAGAAAG |
| eMCK.mmAC | 28 | ccactacgggtttaggctgcccatgtaaggaggcaaggcctggggacac |
| TA2 | | ccgagatgcctggttataattaacccagacatgtggctgcccccccccc |
| | | ccccaacacctgctgcctctaaaaataaccctgtccctggtggatcggt |
| | | acccgggAGTGCCCGACACCCAAATATGGCTTGGGAAGGGCAGCAACAT |
| | | TCCTTCGGGGCGGTGTGGGGAGAGCTCCCGGGACTATATAAAAACCTGT |
| | | GCAAGGGGACAGGCGGTCACACGGACGTGAAGCCTCACTTCCTACCCTC |
| | | GGCACCCAGGGCCAGAGTCAGAGCAGCACTTAGGGTGGAGGTGGGGAGG |
| | | GTGACCTCGAGACCCAGCAAAGAAAG |
| eMCK.mmAC | 29 | ccactacgggtttaggctgcccatgtaaggaggcaaggcctggggacac |
| TA3 | | ccgagatgcctggttataattaacccagacatgtggctgcccccccccc |
| | | ccccaacacctgctgcctctaaaaataaccctgtccctggtggatcggt |
| | | acccgggAGTTGGGAGGGGCAGACAGCTGGGGATACTCTCCATATACGG |
| | | CCTGGTCCGGTCCTAGCTACCTGGGCCAGGGCCAGTCCTCTCCTTCTTT |
| | | GGTCAGTGCAGGAGACCCGGGCGGGGACCCAGGCTGAGAACCAGCCGAA |
| | | GGAAGGGACTCTAGTGCCCGACACCCAAATATGGCTTGGGAAGGGCAGC |
| | | AACATTCCTTCGGGGCGGTGTGGGGAGAGCTCCCGGGACTATATAAAAA |
| | | CCTGTGCAAGGGGACAGGCGGTCACACGGACGTGAAGCCTCACTTCCTA |
| | | CCCTCGGCACCCAGGGCCAGAGTCAGAGCAGCACTTAGGGTGGAGGTGG |
| | | GGAGGGTGACCTCGAGACCCAGCAAAGAAAG |
| eMCK.huACT | 30 | Ccactacgggtttaggctgcccatgtaaggaggcaaggcctggggacac |
| A1 | | ccgagatgcctggttataattaacccagacatgtggctgcccccccccc |
| | | ccccaacacctgctgcctctaaaaataaccctgtccctggtggatcggt |
| | | acccgggGAAGGGCAGCGACATTCCTGCGGGGTGGCGCGGAGGGAATGC |
| | | CCGCGGGCTATATAAAACCTGAGCAGAGGGACAAGCGGCCACCGCAGCG |
| | | GACAGCGCCAAGTGAAGCCTCGCTTCCCCTCCGCGGCGACCAGGGCCCG |
| | | AGCCGAGAGTAGCAGTTGTAGCTACCCGCC |
| eMCK.huACT | 31 | ccactacgggtttaggctgcccatgtaaggaggcaaggcctggggacac |
| A2 | | ccgagatgcctggttataattaacccagacatgtggctgcccccccccc |
| | | ccccaacacctgctgcctctaaaaataaccctgtccctggtggatcggt |
| | | acccgggGTGCCCAACACCCAAATATGGCTCGAGAAGGGCAGCGACATT |
| | | CCTGCGGGGTGGCGCGGAGGGAATGCCCGCGGGCTATATAAAACCTGAG |
| | | CAGAGGGACAAGCGGCCACCGCAGCGGACAGCGCCAAGTGAAGCCTCGC |
| | | TTCCCCTCCGCGGCGACCAGGGCCCGAGCCGAGAGTAGCAGTTGTAGCT |
| | | ACCCGCC |
| eMCK.huACT | 32 | ccactacgggtttaggctgcccatgtaaggaggcaaggcctggggacac |
| A3 | | ccgagatgcctggttataattaacccagacatgtggctgcccccccccc |
| | | ccccaacacctgctgcctctaaaaataaccctgtccctggtggatcggt |
| | | acccgggAGGGGCAAACCCGCTAGGGAGACACTCCATATACGGCCCGGC |
| | | CCGCGTTACCTGGGACCGGGCCAACCCGCTCCTTCTTTGGTCAACGCAG |
| | | GGGACCCGGGCGGGGGCCCAGGCCGCGAACCGGCCGAGGGAGGGGGCTC |
| | | TAGTGCCCAACACCCAAATATGGCTCGAGAAGGGCAGCGACATTCCTGC |
| | | GGGGTGGCGCGGAGGGAATGCCCGCGGGCTATATAAAACCTGAGCAGAG |
| | | GGACAAGCGGCCACCGCAGCGGACAGCGCCAAGTGAAGCCTCGCTTCCC |
| | | CTCCGCGGCGACCAGGGCCCGAGCCGAGAGTAGCAGTTGTAGCTACCCG |
| | | CC |
| mSYN100E.m | 33 | cggccgtccgccctcggcaccattcctcacgacaccCaaatatggcgac |
| mACTA1 | | gggtgaggaatggtggggagttatttttagagcggtgaggaatggtggg |
| | | caggcagcaggtgttgggggagttatttttagagcggggagttattttt |
| | | agagcggtgaggaatggtggacaccCaaatatggcgacgggtgaggaat |
| | | ggtgccgtcgccatatttgggtgtcccgtccgccctcggtacccgggGA |
| | | AGGGCAGCAACATTCCTTCGGGGCGGTGTGGGGAGAGCTCCCGGGACTA |
| | | TATAAAAACCTGTGCAAGGGGACAGGCGGTCACACGGACGTGAAGCCTC |
| | | ACTTCCTACCCTCGGCACCCAGGGCCAGAGTCAGAGCAGCACTTAGGGT |
| | | GGAGGTGGGGAGGGTGACCTCGAGACCCAGCAAAGAAAG |
| mSYN100E.m | 34 | cggccgtccgccctcggcaccattcctcacgacaccCaaatatggcgac |
| mACTA2 | | gggtgaggaatggtggggagttatttttagagcggtgaggaatggtggg |
| | | caggcagcaggtgttgggggagttatttttagagcggggagttattttt |
| | | agagcggtgaggaatggtggacaccCaaatatggcgacgggtgaggaat |
| | | ggtgccgtcgccatatttgggtgtcccgtccgccctcggtacccgggAG |
| | | TGCCCGACACCCAAATATGGCTTGGGAAGGGCAGCAACATTCCTTCGGG |
| | | GCGGTGTGGGGAGAGCTCCCGGGACTATATAAAAACCTGTGCAAGGGGA |
| | | CAGGCGGTCACACGGACGTGAAGCCTCACTTCCTACCCTCGGCACCCAG |
| | | GGCCAGAGTCAGAGCAGCACTTAGGGTGGAGGTGGGGAGGGTGACCTCG |
| | | AGACCCAGCAAAGAAAG |
| mSYN100E.m | 35 | cggccgtccgccctcggcaccattcctcacgacaccCaaatatggcgac |
| mACTA3 | | gggtgaggaatggtggggagttatttttagagcggtgaggaatggtggg |
| | | caggcagcaggtgttgggggagttatttttagagcggggagttattttt |
| | | agagcggtgaggaatggtggacaccCaaatatggcgacgggtgaggaat |
| | | ggtgccgtcgccatatttgggtgtcccgtccgccctcggtacccgggAG |
| | | TTGGGAGGGGCAGACAGCTGGGGATACTCTCCATATACGGCCTGGTCCG |
| | | GTCCTAGCTACCTGGGCCAGGGCCAGTCCTCTCCTTCTTTGGTCAGTGC |
| | | AGGAGACCCGGGCGGGGACCCAGGCTGAGAACCAGCCGAAGGAAGGGAC |
| | | TCTAGTGCCCGACACCCAAATATGGCTTGGGAAGGGCAGCAACATTCCT |
| | | TCGGGGCGGTGTGGGGAGAGCTCCCGGGACTATATAAAAACCTGTGCAA |
| | | GGGGACAGGCGGTCACACGGACGTGAAGCCTCACTTCCTACCCTCGGCA |
| | | CCCAGGGCCAGAGTCAGAGCAGCACTTAGGGTGGAGGTGGGGAGGGTGA |
| | | CCTCGAGACCCAGCAAAGAAAG |
| mSYN100E.hu | 36 | cggccgtccgccctcggcaccattcctcacgacaccCaaatatggcgac |
| ACTA1 | | gggtgaggaatggtggggagttatttttagagcggtgaggaatggtggg |
| | | caggcagcaggtgttgggggagttatttttagagcggggagttattttt |
| | | agagcggtgaggaatggtggacaccCaaatatggcgacgggtgaggaat |
| | | ggtgccgtcgccatatttgggtgtcccgtccgccctcggtacccgggGA |
| | | AGGGCAGCGACATTCCTGCGGGGTGGCGCGGAGGGAATGCCCGCGGGCT |
| | | ATATAAAACCTGAGCAGAGGGACAAGCGGCCACCGCAGCGGACAGCGCC |
| | | AAGTGAAGCCTCGCTTCCCCTCCGCGGCGACCAGGGCCCGAGCCGAGAG |
| | | TAGCAGTTGTAGCTACCCGCC |
| mSYN100E.hu | 37 | cggccgtccgccctcggcaccattcctcacgacaccCaaatatggcgac |
| ACTA2 | | gggtgaggaatggtggggagttatttttagagcggtgaggaatggtggg |
| | | caggcagcaggtgttgggggagttatttttagagcggggagttattttt |
| | | agagcggtgaggaatggtggacaccCaaatatggcgacgggtgaggaat |
| | | ggtgccgtcgccatatttgggtgtcccgtccgccctcggtacccgggGT |
| | | GCCCAACACCCAAATATGGCTCGAGAAGGGCAGCGACATTCCTGCGGGG |
| | | TGGCGCGGAGGGAATGCCCGCGGGCTATATAAAACCTGAGCAGAGGGAC |
| | | AAGCGGCCACCGCAGCGGACAGCGCCAAGTGAAGCCTCGCTTCCCCTCC |
| | | GCGGCGACCAGGGCCCGAGCCGAGAGTAGCAGTTGTAGCTACCCGCC |
| mSYN100E.hu | 38 | cggccgtccgccctcggcaccattcctcacgacaccCaaatatggcgac |
| ACTA3 | | gggtgaggaatggtggggagttatttttagagcggtgaggaatggtggg |
| | | caggcagcaggtgttgggggagttatttttagagcggggagttattttt |
| | | agagcggtgaggaatggtggacaccCaaatatggcgacgggtgaggaat |
| | | ggtgccgtcgccatatttgggtgtcccgtccgccctcggtacccgggAG |
| | | GGGCAAACCCGCTAGGGAGACACTCCATATACGGCCCGGCCCGCGTTAC |
| | | CTGGGACCGGGCCAACCCGCTCCTTCTTTGGTCAACGCAGGGGACCCGG |
| | | GCGGGGGCCCAGGCCGCGAACCGGCCGAGGGAGGGGGCTCTAGTGCCCA |
| | | ACACCCAAATATGGCTCGAGAAGGGCAGCGACATTCCTGCGGGGTGGCG |
| | | CGGAGGGAATGCCCGCGGGCTATATAAAACCTGAGCAGAGGGACAAGCG |
| | | GCCACCGCAGCGGACAGCGCCAAGTGAAGCCTCGCTTCCCCTCCGCGGC |
| | | GACCAGGGCCCGAGCCGAGAGTAGCAGTTGTAGCTACCCGCC |
| eMCK.mmAC | 39 | ccactacgggtttaggctgcccatgtaaggaggcaaggcctggggacac |
| TA2shortUTR | | ccgagatgcctggttataattaacccagacatgtggctgcccccccccc |
| | | ccccaacacctgctgcctctaaaaataaccctgtccctggtggatcggt |
| | | acccgggAGTGCCCGACACCCAAATATGGCTTGGGAAGGGCAGCAACAT |
| | | TCCTTCGGGGCGGTGTGGGGAGAGCTCCCGGGACTATATAAAAACCTGT |
| | | GCAAGGGGACAGGCGGTCACACGGACGTGAAGCCT |
| eMCK.huACT | 40 | ccactacgggtttaggctgcccatgtaaggaggcaaggcctggggacac |
| A2shortUTR | | ccgagatgcctggttataattaacccagacatgtggctgcccccccccc |
| | | ccccaacacctgctgcctctaaaaataaccctgtccctggtggatcggt |
| | | acccgggGTGCCCAACACCCAAATATGGCTCGAGAAGGGCAGCGACATT |
| | | CCTGCGGGGTGGCGCGGAGGGAATGCCCGCGGGCTATATAAAACCTGAG |
| | | CAGAGGGACAAGCGGCCACCGCAGCGGACAG |
| SPC5v2.mmA | 41 | GGCCGTCCGCCCTCGGCACCATCCTCACGACACCCAAATATGGCGACGG |
| CTA2shortUT | | GTGAGGAATGGTGGGGAGTTATTTTTAGAGCGGTGAGGAAGGTGGGCAG |
| R | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGTGCCCGACACCCAAAT |
| | | ATGGCTTGGGAAGGGCAGCAACATTCCTTCGGGGCGGTGTGGGGAGAGC |
| | | TCCCGGGACTATATAAAAACCTGTGCAAGGGGACAGGCGGTCACACGGA |
| | | CGTGAAGCCT |
| eMCK.SPC5v | 42 | ccactacgggtttaggctgcccatgtaaggaggcaaggcctggggacac |
| 2.mmACTA2s | | ccgagatgcctggttataattaacccagacatgtggctgcccccccccc |
| hortUTR | | ccccaacacctgctgcctctaaaaataaccctgtccctggtggatcGTC |
| | | GGCCGTCCGCCCTCGGCACCATCCTCACGACACCCAAATATGGCGACGG |
| | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGTGCCCGACACCCAAAT |
| | | ATGGCTTGGGAAGGGCAGCAACATTCCTTCGGGGCGGTGTGGGGAGAGC |
| | | TCCCGGGACTATATAAAAACCTGTGCAAGGGGACAGGCGGTCACACGGA |
| | | CGTGAAGCCT |
| seMCK.SPC5v | 68 | tgcctggttataattaacccagacatgtggctgccccccccccccccaa |
| 2.mmACTA2s | | cacctgctgcctctaaaaataaccctgtccctggtggatcGTCGGCCGT |
| hortUTR | | CCGCCCTCGGCACCATCCTCACGACACCCAAATATGGCGACGGGTGAGG |
| | | AATGGTGGGGAGTTATTTTTAGAGCGGTGAGGAAGGTGGGCAGGCAGCA |
| | | GGTGTTGGGGGAGTTATTTTTAGAGCGGGGAGTTATTTTTAGAGCGGAG |
| | | GAATGGTGGACACCCAAATATGGCGACGGTTCCTCACGGACACCCAAAT |
| | | ATGGCGACGGGCCCTggtacccgggAGTGCCCGACACCCAAATATGGCT |
| | | TGGGAAGGGCAGCAACATTCCTTCGGGGCGGTGTGGGGAGAGCTCCCGG |
| | | GACTATATAAAAACCTGTGCAAGGGGACAGGCGGTCACACGGACGTGAA |
| | | GCCT |
| eMCK.SPC5v | 43 | ccactacgggtttaggctgcccatgtaaggaggcaaggcctggggacac |
| 2.mmACTA2 | | ccgagatgcctggttataattaacccagacatgtggctgcccccccccc |
| midUTR | | ccccaacacctgctgcctctaaaaataaccctgtccctggtggatcGTC |
| | | GGCCGTCCGCCCTCGGCACCATCCTCACGACACCCAAATATGGCGACGG |
| | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGTGCCCGACACCCAAAT |
| | | ATGGCTTGGGAAGGGCAGCAACATTCCTTCGGGGCGGTGTGGGGAGAGC |
| | | TCCCGGGACTATATAAAAACCTGTGCAAGGGGACAGGCGGTCACACGGA |
| | | CGTGAAGCCTCACTTCCTACCCTCGGCACCCAGGGCCAGAGTCAGAGCA |
| spc5v2.mmAC | 44 | GGCCGTCCGCCCTCGGCACCATCCTCACGACACCCAAATATGGCGACGG |
| TA3shortUTR | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGTTGGGAGGGGCAGACA |
| | | GCTGGGGATACTCTCCATATACGGCCTGGTCCGGTCCTAGCTACCTGGG |
| | | CCAGGGCCAGTCCTCTCCTTCTTTGGTCAGTGCAGGAGACCCGGGCGGG |
| | | GACCCAGGCTGAGAACCAGCCGAAGGAAGGGACTCTAGTGCCCGACACC |
| | | CAAATATGGCTTGGGAAGGGCAGCAACATTCCTTCGGGGCGGTGTGGGG |
| | | AGAGCTCCCGGGACTATATAAAAACCTGTGCAAGGGGACAGGCGGTCAC |
| | | ACGGACGTGAAGCCT |
| spc5v2.mmAC | 45 | GGCCGTCCGCCCTCGGCACCATCCTCACGACACCCAAATATGGCGACGG |
| TA4 | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGTTGGGAGGGGCAGACA |
| | | GCTGGGGATACTCTCCATATACGGCCTGGTCCGGTCCAGTGCCCGACAC |
| | | CCAAATATGGCTTGGGAAGGGCAGCAACATTCCTTCGGGGCGGTGTGGG |
| | | GAGAGCTCCCGGGACTATATAAAAACCTGTGCAAGGGGACAGGCGGTCA |
| | | CACGGACGTGAAGCCT |
| eMCK.spc5v2. | 46 | GGCCGTCCGCCCTCGGCACCATCCTCACGACACCCAAATATGGCGACGG |
| mmACTA4 | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGTTGGGAGGGGCAGACA |
| | | GCTGGGGATACTCTCCATATACGGCCTGGTCCGGTCCAGTGCCCGACAC |
| | | CCAAATATGGCTTGGGAAGGGCAGCAACATTCCTTCGGGGCGGTGTGGG |
| | | GAGAGCTCCCGGGACTATATAAAAACCTGTGCAAGGGGACAGGCGGTCA |
| | | CACGGACGTGAAGCCT |
| SPc5v2.huAC | 47 | GGCCGTCCGCCCTCGGCACCATCCTCACGACACCCAAATATGGCGACGG |
| TA3shortUTR | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGGGGCAAACCCGCTAGG |
| | | GAGACACTCCATATACGGCCCGGCCCGCGTTACCTGGGACCGGGCCAAC |
| | | CCGCTCCTTCTTTGGTCAACGCAGGGGACCCGGGCGGGGGCCCAGGCCG |
| | | CGAACCGGCCGAGGGAGGGGGCTCTAGTGCCCAACACCCAAATATGGCT |
| | | CGAGAAGGGCAGCGACATTCCTGCGGGGTGGCGCGGAGGGAATGCCCGC |
| | | GGGCTATATAAAACCTGAGCAGAGGGACAAGCGGCCACCGCAGCGGACA |
| | | GCGCCAAGTGAAG |
| SPc5v2.huAC | 48 | GGCCGTCCGCCCTCGGCACCATCCTCACGACACCCAAATATGGCGACGG |
| TA3midUTR | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGGGGCAAACCCGCTAGG |
| | | GAGACACTCCATATACGGCCCGGCCCGCGTTACCTGGGACCGGGCCAAC |
| | | CCGCTCCTTCTTTGGTCAACGCAGGGGACCCGGGGGGGGGCCCAGGCCG |
| | | CGAACCGGCCGAGGGAGGGGGCTCTAGTGCCCAACACCCAAATATGGCT |
| | | CGAGAAGGGCAGCGACATTCCTGCGGGGTGGCGCGGAGGGAATGCCCGC |
| | | GGGCTATATAAAACCTGAGCAGAGGGACAAGCGGCCACCGCAGCGGACA |
| | | GCGCCAAGTGAAGCCTCGCTTCCCCTCCGCGGCGACCA |
| SPc5v2.huAC | 49 | GGCCGTCCGCCCTCGGCACCATCCTCACGACACCCAAATATGGCGACGG |
| TA2midUTR | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggGTGCCCAACACCCAAATA |
| | | TGGCTCGAGAAGGGCAGCGACATTCCTGCGGGGTGGCGCGGAGGGAATG |
| | | CCCGCGGGCTATATAAAACCTGAGCAGAGGGACAAGCGGCCACCGCAGC |
| | | GGACAGCGCCAAGTGAAGCCTCGCTTCCCCTCCGCGGCGACCA |
| eMCK.SPC5v | 50 | ccactacgggtttaggctgcccatgtaaggaggcaaggcctggggacac |
| 2.huACTA3mi | | ccgagatgcctggttataattaacccagacatgtggctgcccccccccc |
| dUTR | | ccccaacacctgctgcctctaaaaataaccctgtccctggtggatcGTC |
| | | GGCCGTCCGCCCTCGGCACCATCCTCACGACACCCAAATATGGCGACGG |
| | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGGGGCAAACCCGCTAGG |
| | | GAGACACTCCATATACGGCCCGGCCCGCGTTACCTGGGACCGGGCCAAC |
| | | CCGCTCCTTCTTTGGTCAACGCAGGGGACCCGGGCGGGGGCCCAGGCCG |
| | | CGAACCGGCCGAGGGAGGGGGCTCTAGTGCCCAACACCCAAATATGGCT |
| | | CGAGAAGGGCAGCGACATTCCTGCGGGGTGGCGCGGAGGGAATGCCCGC |
| | | GGGCTATATAAAACCTGAGCAGAGGGACAAGCGGCCACCGCAGCGGACA |
| | | GCGCCAAGTGAAGCCTCGCTTCCCCTCCGCGGCGACCA |
| eMCK.SPC5v | 51 | ccactacgggtttaggctgcccatgtaaggaggcaaggcctggggacac |
| 2.huACTA2mi | | ccgagatgcctggttataattaacccagacatgtggctgcccccccccc |
| dUTR | | ccccaacacctgctgcctctaaaaataaccctgtccctggtggatcGTC |
| | | GGCCGTCCGCCCTCGGCACCATCCTCACGACACCCAAATATGGCGACGG |
| | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggGTGCCCAACACCCAAATA |
| | | TGGCTCGAGAAGGGCAGCGACATTCCTGCGGGGTGGCGCGGAGGGAATG |
| | | CCCGCGGGCTATATAAAACCTGAGCAGAGGGACAAGCGGCCACCGCAGC |
| | | GGACAGCGCCAAGTGAAGCCTCGCTTCCCCTCCGCGGCGACCA |
| SPc5v2.huAC | 52 | GGCCGTCCGCCCTCGGCACCATCCTCACGACACCCAAATATGGCGACGG |
| TA4 | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGGGGCAAACCCGCTAGG |
| | | GAGACACTCCATATACGGCCCGGCCCGCGTTACCTGGGTGCCCAACACC |
| | | CAAATATGGCTCGAGAAGGGCAGCGACATTCCTGCGGGGTGGCGCGGAG |
| | | GGAATGCCCGCGGGCTATATAAAACCTGAGCAGAGGGACAAGCGGCCAC |
| | | CGCAGCGGACAGCGCCAAGTGAAGCCTCGCTTCCCCTCCGCGGCGACCA |
| SPc5v2.huAC | 53 | GGCCGTCCGCCCTCGGCACCATCCTCACGACACCCAAATATGGCGACGG |
| TA3 | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGGGGCAAACCCGCTAGG |
| | | GAGACACTCCATATACGGCCCGGCCCGCGTTACCTGGGACCGGGCCAAC |
| | | CCGCTCCTTCTTTGGTCAACGCAGGGGACCCGGGCGGGGGCCCAGGCCG |
| | | CGAACCGGCCGAGGGAGGGGGCTCTAGTGCCCAACACCCAAATATGGCT |
| | | CGAGAAGGGCAGCGACATTCCTGCGGGGTGGCGCGGAGGGAATGCCCGC |
| | | GGGCTATATAAAACCTGAGCAGAGGGACAAGCGGCCACCGCAGCGGACA |
| | | GCGCCAAGTGAAGCCTCGCTTCCCCTCCGCGGCGACCAGGGCCCGAGCC |
| | | GAGAGTAGCAGTTGTAGCTACCCGCC |
| SPc5v2.mmA | 54 | GGCCGTCCGCCCTCGGCACCATCCTCACGACACCCAAATATGGCGACGG |
| CTA3 | | GTGAGGAATGGTGGGGAGTTATTTTTAGAGCGGTGAGGAAGGTGGGCAG |
| | | GCAGCAGGTGTTGGGGGAGTTATTTTTAGAGCGGGGAGTTATTTTTAGA |
| | | GCGGAGGAATGGTGGACACCCAAATATGGCGACGGTTCCTCACGGACAC |
| | | CCAAATATGGCGACGGGCCCTggtacccgggAGTTGGGAGGGGCAGACA |
| | | GCTGGGGATACTCTCCATATACGGCCTGGTCCGGTCCTAGCTACCTGGG |
| | | CCAGGGCCAGTCCTCTCCTTCTTTGGTCAGTGCAGGAGACCCGGGCGGG |
| | | GACCCAGGCTGAGAACCAGCCGAAGGAAGGGACTCTAGTGCCCGACACC |
| | | CAAATATGGCTTGGGAAGGGCAGCAACATTCCTTCGGGGCGGTGTGGGG |
| | | AGAGCTCCCGGGACTATATAAAAACCTGTGCAAGGGGACAGGCGGTCAC |
| | | ACGGACGTGAAGCCTCACTTCCTACCCTCGGCACCCAGGGCCAGAGTCA |
| | | GAGCAGCACTTAGGGTGGAGGTGGGGAGGGTGACCTCGAGACCCAGCAA |
| | | AGAAAG |
| Alpha-Mic/Bik | 55 | aggttaatttttaaaaagcagtcaaaagtccaagtggcccttggcagca |
| Enhancer | | tttactctctctgtttgctctggttaataatctcaggagcacaaacatt |
| (Mic/BikE) | | CC |
| Tandem (2) | 56 | aggttaatttttaaaaagcagtcaaaagtccaagtggcccttggcagca |
| alpha-Mic/Bik | | tttactctctctgtttgctctggttaataatctcaggagcacaaacatt |
| Enhancers | | ccaggttaatttttaaaaagcagtcaaaagtccaagtggcccttggcag |
| (2 Mic/BikE) | | catttactctctctgtttgctctggttaataatctcaggagcacaaaca |
| | | ttcc |
| Tandem (2) | 57 | ccactacgggtttaggctgcccatgtaaggaggcaaggcctggggacac |
| Mck | | ccgagatgcctggttataattaacccagacatgtggctgcccccccccc |
| Enhancers | | ccccaacacctgctgcctctaaaaataaccctgtccctggtggatccca |
| | | ctacgggtttaggctgcccatgtaaggaggcaaggcctggggacacccg |
| | | agatgcctggttataattaacccagacatgtggctgccccccccccccc |
| | | caacacctgctgcctctaaaaataaccctgtccctggtggatc |
| Tandem Mck | 58 | ccactacgggtttaggctgcccatgtaaggaggcaaggcctggggacac |
| (3) Enhancers | | ccgagatgcctggttataattaacccagacatgtggctgcccccccccc |
| | | ccccaacacctgctgcctctaaaaataaccctgtccctggtggatccca |
| | | ctacgggtttaggctgcccatgtaaggaggcaaggcctggggacacccg |
| | | agatgcctggttataattaacccagacatgtggctgccccccccccccc |
| | | caacacctgctgcctctaaaaataaccctgtccctggtggatcccacta |
| | | cgggtttaggctgcccatgtaaggaggcaaggcctggggacacccgaga |
| | | tgcctggttataattaacccagacatgtggctgccccccccccccccaa |
| | | cacctgctgcctctaaaaataaccctgtccctggtggatc |
| Myosin heavy | 59 | cccttcagattaaaaataactgaggtaagggcctgggtaggggaggtgg |
| chain enhancer | | tgtgagacgctcctgtctctcctctatctgcccatcggccctttgggga |
| (MhcE) | | ggaggaatgtgcccaaggactaaaaaaaggccatggagccagaggggcg |
| | | agggcaacagacctttcatgggcaaaccttggggccctgctgaagcttt |
| | | ggc |
| CK8 Promoter | 60 | ccctgcatgcgaagatcttcgaacaaggctgtgggggactgagggcagg |
| | | ctgtaacaggcttgggggccagggcttatacgtgcctgggactcccaaa |
| | | gtattactgttccatgttcccggcgaagggccagctgtcccccgccagc |
| | | tagactcagcacttagtttaggaaccagtgagcaagtcagcccttgggg |
| | | cagcccatacaaggccatggggctgggcaagctgcacgcctgggtccgg |
| | | ggtgggcacggtgcccgggcaacgagctgaaagctcatctgctctcagg |
| | | ggcccctccctggggacagcccctcctggctagtcacaccctgtaggct |
| | | cctctatataacccaggggcacaggggctgccctcattctaccaccacc |
| | | tccacagcacagacagacactcaggagccagccagcgtcga |
| Minimal | 61 | gaatggtggacacccaaatatggcgacggttcctcacccgtcgccatat |
| SPc5.12 | | ttgggtgtccgccctcggccggggccgcattcctgggggccgggcggtg |
| | | ctcccgcccgcctcgataaaaggctccggggccggcggcggcccacgag |
| | | ctacccggaggagcgggaggcgccaag |
| Promoter | 62 | gacattgattattgactagttattaatagtaatcaattacggggtcatt |
| CAG Promoter | | agttcatagcccatatatggagttccgcgttacataacttacggtaaat |
| | | ggcccgcctggctgaccgcccaacgacccccgcccattgacgtcaataa |
| | | tgacgtatgttcccatagtaacgccaatagggactttccattgacgtca |
| | | atgggtggagtatttacggtaaactgcccacttggcagtacatcaagtg |
| | | tatcatatgccaagtacgccccctattgacgtcaatgacggtaaatggc |
| | | ccgcctggcattatgcccagtacatgaccttatgggactttcctacttg |
| | | gcagtacatctacgtattagtcatcgctattaccatggtcgaggtgagc |
| | | cccacgttctgcttcactctccccatctcccccccctccccacccccaa |
| | | ttttgtatttatttattttttaattattttgtgcagcgatgggggcggg |
| | | ggcgggggggggggcgcgcgccaggcggggggggcggggcgaggggcgg |
| | | ggcggggcgaggcggagaggtgcggcggcagccaatcagagcggcgcgc |
| | | tccgaaagtttccttttatggcgaggcggcggcggcggcggccctataa |
| | | aaagcgaagcgcgcggcgggcgggagtcgctgcgcgctgccttcgcccc |
| | | gtgccccgctccgccgccgcc-cgcgccgcccgccccggctctgactga |
| | | ccgcgttactcccacaggtgagcgggcgggacggcccttctcctccggg |
| | | ctgtaattagcgcttggtttaatgacggcttgtttcttttctgtggctg |
| | | cgtgaaagccttgaggggctccgggagggccctttgtgcggggggagcg |
| | | gctcggggggtgcgtgcgtgtgtgtgtgcgtggggagcgccgcgtgcgg |
| | | ctccgcgctgcccggcggctg gagcgctgcgggcgcggcgcggggctt |
| | | tgtgcgctccgcagtgtgcgcgaggggagcgcggccgggggcggtgccc |
| | | cgcggtgcggggggggctgcgaggggaacaaaggctgcgtgcggggtgt |
| | | gtgcgtgggggggtgagcagggggtgtgggcgcgtcggtcgggctgcaa |
| | | ccccccctgcacccccctccccgagttgctgagcacggcccggcttcgg |
| | | gtgcggggctccgtacggggcgtggcgcggggctcgccgtgccgggcgg |
| | | ggggtggcggcaggtgggggtgccgggcggggcggggccgcctcgggcc |
| | | ggggagggctcgggggaggggcgcggcggcccccggagcgccggcggct |
| | | gtcgaggcgcggcgagccgcagccattgccttttatggtaatcgtgcga |
| | | gagggcgcagggacttcctttgtcccaaatctgtgcggagccgaaatct |
| | | gggaggcgccgccgcaccccctctagcgggcgcggggcgaagcggtgcg |
| | | gcgccggcaggaaggaaatgggcggggagggccttcgtgcgtcgccgcg |
| | | ccgccgtccccttctccctctccagcctcggggctgtccgcggggggac |
| | | ggctgccttcgggggggacggggcagggcggggttcggcttctggcgtg |
| | | tgaccggcggctctagagcctctgctaaccatgttcatgccttcttctt |
| | | tttcctacag |
| eMCK.fSPC5v | 69 | ccactacgggtttaggctgcccatgtaaggaggcaaggcctggggacac |
| 2.MMACTA2s | | ccgagatgcctggttataattaacccagacatgtggctgcccccccccc |
| hortUTR | | ccccaacacctgctgcctctaaaaataaccctgtccctggtggatcTGA |
| | | GGAAGGTGGGCAGGCAGCAGGTGTTGGGGGAGTTATTTTTAGAGCGGGG |
| | | AGTTATTTTTAGAGCGGAGGAATGGTGGACACCCAAATATGGCGACGGT |
| | | TCCTCACGGACACCCAAATATGGCGACGGGCCCTggtacccgggAGTGC |
| | | CCGACACCCAAATATGGCTTGGGAAGGGCAGCAACATTCCTTCGGGGCG |
| | | GTGTGGGGAGAGCTCCCGGGACTATATAAAAACCTGTGCAAGGGGACAG |
| | | GCGGTCACACGGACGTGAAGCCT |
| fSPC5v2 | 70 | TGAGGAAGGTGGGCAGGCAGCAGGTGTTGGGGGAGTTATTTTTAGAGCG |
| (fragmented | | GGGAGTTATTTTTAGAGCGGAGGAATGGTGGACACCCAAATATGGCGAC |
| SPC5v2) | | GGTTCCTCACGGACACCCAAATATGGCGACGGGCCCT |

### 5.2.1 Enhancers

The present inventors have surprisingly discovered multiple enhancers are amenable to tandem positioning while operably linked to one or more promoters. These enhancers when arranged in tandem and operably linked to promoters and a transgene promote tissue specific expression of the transgenes.

Accordingly, provided are muscle creatine kinase (MCK) enhancers, particularly an eMCK Enhancer (modified MCK enhancer) or seMCK Enhancer (short modified MCK enhancer), as in SEQ ID NO: 4 or 5. Also provided is mSYN100E (SEQ ID NO: 6). Also provided are muscle specific enhancers, such as, Mus022, (SEQ ID NO: 10). In embodiments, enhancers increase muscle specific expression, such muscle CREs, including Mus022 (SEQ ID NO: 10), and also including. Mus007 (SEQ ID NO: 11), Mus011 (SEQ ID NO: 12) and Mus035 (SEQ ID NO: 13).

In embodiments, the enhancer comprises the nucleic acid sequence of SEQ ID NO: 4, or a sequence that is 99%, 95%, 90%, 85% or 80% identical to SEQ ID NO: 4 and/or comprises 1 to 10 nucleic acid substitutions while retaining biological activity of the enhancer. In embodiments, the enhancer comprises the nucleic acid sequence of SEQ ID NO: 5, or a sequence that is 99%, 95%, 90%, 85% or 80% identical to SEQ ID NO: 5 and/or comprises 1 to 10 nucleic acid substitutions while retaining biological activity of the enhancer. In embodiments, the enhancer comprises the nucleic acid sequence of SEQ ID NO: 6, or a sequence that is 99%, 95%, 90%, 85% or 80% identical to SEQ ID NO: 6 and/or comprises 1 to 10 nucleic acid substitutions while retaining biological activity of the enhancer. In embodiments, the enhancer comprises the nucleic acid sequence of SEQ ID NO: 10, or a sequence that is 99%, 95%, 90%, 85% or 80% identical to SEQ ID NO: 10 and/or comprises 1 to 10 nucleic acid substitutions while retaining biological activity of the enhancer. In embodiments, the enhancer comprises the nucleic acid sequence of SEQ ID NO: 11, or a sequence that is 99%, 95%, 90%, 85% or 80% identical to SEQ ID NO: 11 and/or comprises 1 to 10 nucleic acid substitutions while retaining biological activity of the enhancer. In embodiments, the enhancer comprises the nucleic acid sequence of SEQ ID NO: 12, or a sequence that is 99%, 95%, 90%, 85% or 80% identical to SEQ ID NO: 12 and/or comprises 1 to 10 nucleic acid substitutions while retaining biological activity of the enhancer. In embodiments, the enhancer comprises the nucleic acid sequence of SEQ ID NO: 13, or a sequence that is 99%, 95%, 90%, 85% or 80% identical to SEQ ID NO: 13 and/or comprises 1 to 10 nucleic acid substitutions while retaining biological activity of the enhancer.

In embodiments, the enhancer element comprises or consists of one or more of SEQ ID NOs: 4, 5, 6, 10, 11, 12 or 13.

Other enhancers are well known to the skilled person in the art.

### 5.2.2 Promoters

Another aspect of the present invention relates to nucleic acid expression cassettes comprising chimeric regulatory elements designed to confer or enhance liver-specific and muscle-specific expression (including skeletal o muscle specific expression). The invention involves engineering regulatory elements in tandem, including promoter elements, enhancer elements, and optionally introns. Examples include but are not limited to Spc5-12, or variant thereof, such as SPc5v2 (See WO 2023/178053, which is incorporated herein by reference in its entirety), or syn100 promoters (such as SEQ ID NO: 2, 3, 7 and 61), CK promoter (SEQ ID NO: 8), and ACTA core promoters (SEQ ID NOs: 14-26).

Provided are promoter elements that enhance gene expression in the skeletal muscle which have 99%, 95%, 90%, 85% or 80% sequence identity with any one of SEQ ID NOs: 2, 3, 7, 14-26 and 61 and/or comprise 1 to 10 nucleic acid substitutions while retaining biological activity of the promoter. In embodiments, the promtoer element comprises or consists of one or more of SEQ ID NOs: 2, 3, 7, 14-26 and 61.

The unique combinations of promoter and enhancer sequences provided herein improve transgene expression while maintaining tissue specificity. The novel regulatory element nucleic acids were generated using a method to improve transgene expression from tandem enhancer/promoter combinations while reducing expression in undesirable cells or tissues and reducing the size of the promoter in the gene cassette. Ultimately, these designs aim to improve the therapeutic efficacy of gene transfer by providing more robust levels of transgene expression, improved stability/persistence, and lower dosing via AAV delivery.

The CAG promoter (SEQ ID NO: 17) refers to a chimeric promoter constructed from the following sequences: the cytomegalovirus (CMV) early enhancer element (C), the chicken beta-actin promoter (the first exon and the first intron of chicken beta-actin gene) (A), and the splice acceptor of the rabbit beta-globin gene (G).The CAG promoter is frequently utilized in the art to drive high levels of expression in mammalian cells, and is non-preferential with respect to tissue specificity, therefore is typically utilized as a universal promoter. Other promoters that may be used in combination with the regulatory elements herein are well known to the skilled person in the art.

### 5.2.3 Introns

Another aspect of the present invention relates to nucleic acid expression cassettes comprising an intron within the regulatory cassette. In some embodiments, the intron nucleic acid is a chimeric intron derived from human β-globin and Ig heavy chain (also known as β-globin splice donor/immunoglobulin heavy chain splice acceptor intron, or β-globin/IgG chimeric intron, Reed. R., et al. Genes and Development. 1989). Use of an intron may further induce efficient splicing in eukaryotic cells. Although use of an intron may not indicate increases in expression to an already strong promoter, the presence of an intron may increase the expression level of transgene and can also increase the duration of expression in vivo.

In some embodiments, the intron is a VH4 intron. The VH4 intron nucleic acid can comprise SEQ ID NO: 64 as shown in Table 2 below. The VH4 intron 5′ of the coding sequence may enhance proper splicing and, thus, transgene expression. Accordingly, in some embodiments, an intron is coupled to the 5′ end of a transgene sequence. In other embodiments, the intron is less than 100 nucleotides in length.

TABLE 2
for different introns

| | SEQ | |
|---|---|---|
| Structure | ID | Sequence |
| Chimeric | 63 | GTAAGTATCAAGGTTACAAGAC |
| intron | | AGGTTTAAGGAGACCAATAGAA |
| (β-globin/ | | ACTGGGCTTGTCGAGACAGAGA |
| Ig | | AGACTCTTGCGTTTCTGATAGG |
| Intron) | | CACCTATTGGTCTTACTGACAT |
| | | CCACTTTGCCTTTCTCTCCACA |
| | | G |
| VH4 intron | 64 | GTGAGTATCTCAGGGATCCAGA |
| | | CATGGGGATATGGGAGGTGCCT |
| | | CTGATCCCAGGGCTCACTGTGG |
| | | GTCTCTCTGTTCACAG |
| SV40 intron | 65 | GTAAGTTTAGTCTTTTTGTCTT |
| | | TTATTTCAGGTCCCGGATCCGG |
| | | TGGTGGTGCAAATCAAAGAACT |
| | | GCTCCTCAGTGGATGTTGCCTT |
| | | TACTTCTAG |

In other embodiments, the intron is a chimeric intron derived from human β-globin and Ig heavy chain (also known as β-globin splice donor/immunoglobulin heavy chain splice acceptor intron, or β-globin/IgG chimeric intron) (Table 2, SEQ ID NO: 63). Other introns well known to the skilled person may be employed, such as the chicken β-actin intron, minute virus of mice (MVM) intron, human factor IX intron (e.g., FIX truncated intron 1), β-globin splice donor/immunoglobulin heavy chain splice acceptor intron, adenovirus splice donor/immunoglobulin splice acceptor intron, SV40 late splice donor/splice acceptor (19S/16S) intron (Table 2, SEQ ID NO: 65).

Other introns well known to the skilled person may be employed.

### 5.2.4 Other Regulatory Elements

5.2.4.1 polyA

Another aspect of the present disclosure relates to expression cassettes comprising a polyadenylation (polyA) site downstream of the coding region of the transgene. Any polyA site that signals termination of transcription and directs the synthesis of a polvA tail is suitable for use in AAV vectors of the present disclosure. Exemplary polyA signals are derived from, but not limited to, the following: the SV40 late gene, the rabbit β-globin gene (SEQ ID NO: 67), the bovine growth hormone (BPH) gene, the human growth hormone (hGH) gene, the synthetic polyA (SPA) site, and the bovine growth hormone (bGH) gene. See, e.g., Powell and Rivera-Soto, 2015, Discov. Med., 19(102):49-57. In one embodiment, the polyA signal comprises SEQ ID NO: 66 as shown in Table 3.

TABLE 3
Nucleotide Sequence of the PolyA Signal

| | SEQ ID | |
|---|---|---|
| Structure | NO: | Sequence |
| β-globin | 66 | ataaaggaaatttattttcatt |
| Poly A | | gcaatagtgtgttggaattttt |
| signal | | tgtgtctctca |
| Rabbit | 67 | gatctttttccctctgccaaaa |
| β-globin | | attatggggacatcatgaagcc |
| poly A | | ccttgagcatctgacttctggc |
| | | taataaaggaaatttattttca |
| | | ttgcaatagtgtgttggaattt |
| | | tttgtgtctctcactcg |

### 5.3. Vectors for Gene Delivery

Another aspect of the present invention relates to the genetic engineering of tandem nucleic acid regulatory elements and incorporating these nucleic acid sequences in a vector expression system. In one embodiment, the vector is a viral vector, including but not limited to recombinant adeno-associated viral (rAAV) vectors (e.g. Gao G., et al 2003 Proc. Natl. Acad. Sci. U.S.A. 100(10):6081-6086), lentiviral vectors (e.g. Matrai, J, et al. 2011. Hepatology 53, 1696-707), retroviral vectors (e.g. Axelrod, J H, et al. 1990. Proc Natl Acad Sci USA; 87, 5173-7), adenoviral vectors (e.g. Brown et al., 2004 Blood 103, 804-10), herpes-simplex viral vectors (Marconi, P. et al. Proc Natl Acad Sci USA. 1996 93(21): 11319-11320; Baez, M V, et al. Chapter 19—Using Herpes Simplex Virus Type 1-Based Amplicon Vectors for Neuroscience Research and Gene Therapy of Neurologic Diseases, Ed.: Robert T. Gerlai, Molecular-Genetic and Statistical Techniques for Behavioral and Neural Research, Academic Press, 2018:Pages 445-477), and retrotransposon-based vector systems (e.g. Soifer, 2004, Current Gene Therapy 4(4):373-384). In another embodiment, the vector is a non-viral vector. rAAV vectors have limited packaging capacity of the vector particles (i.e. approximately 4.7 kb), constraining the size of the transgene expression cassette to obtain functional vectors (Jiang et al., 2006 Blood. 108:107-15). The length of the transgene and the length of the regulatory nucleic acid sequences comprising tandem enhancer(s) and promoter(s) are taken into consideration when selecting a regulatory region suitable for a particular transgene and target tissue.

Another aspect of the present invention relates to a viral vector comprising an expression cassette comprising a nucleic acid regulatory element described herein, operably linked to a transgene. In some embodiments, the expression cassette comprises a nucleic acid regulatory element comprising the nucleic acid sequence of SEQ ID NO: 9, or a sequence that is 99%, 95%, 90%, 85% or 80% identical to SEQ ID NO: 9 and enhances expression of the transgene in skeletal muscle (with, in embodiments, minimal or reduced expression in cardiac tissue).

In another aspect, the expression cassettes are suitable for packaging in an AAV capsid, as such the cassette comprises (1) AAV inverted terminal repeats (ITRs) flank the expression cassette; (2) regulatory control elements, a) promoter/enhancers, such as any one of the promoters described in Table 1, b) a poly A signal, and c) optionally an intron; and (3) a transgene providing (e.g., coding for) one or more RNA or protein products of interest. In certain embodiments, the transgene is from Tables 4A, 4B or 4C. In a further aspect, the expression cassettes are suitable for packaging as an ssAAV vector or an scAAV vector.

### 5.3.1 AAV

Another aspect of the present invention relates to expression cassettes suitable for packaging in an AAV capsid, as such the cassette comprises (1) AAV inverted terminal repeats (ITRs) flank the expression cassette; (2) regulatory control elements, consisting essentially of one or more enhancers and one or more promoters, particularly one of the muscle-specific regulatory elements provided herein, including a regulatory element or composite enhancer/promoter of Table 1, d) a poly A signal, and e) optionally, an intron; and (3) a transgene providing (e.g., coding for) one or more RNA or protein products of interest.

The provided nucleic acids and methods are suitable for use in the production of any isolated recombinant AAV particles, in the production of a composition comprising any isolated recombinant AAV particles, or in the method for treating a disease or disorder in a subject in need thereof comprising the administration of any isolated recombinant AAV particles. As such, the rAAV may be of any serotype, modification, or derivative, known in the art, or any combination thereof (e.g., a population of rAAV particles that comprises two or more serotypes, e.g., comprising two or more of rAAV2, rAAV8, and rAAV9 particles) known in the art. In some embodiments, the rAAV particles are AAV1, AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6. AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof.

In some embodiments, rAAV particles have a capsid protein from an AAV serotype selected from AAV1, AAV1, AAV2, rAAV3. AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV2.5, AAV2tYF, AAV3B, AAV.LK03, AAV.HSC1, AAV.HSC2. AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.hu32, AAV.Anc80, rAAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise a capsid protein from an AAV capsid serotype selected from AAV1. AAV1. AAV2, rAAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, rAAV particles comprise a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8. AAV9, AAV10, AAV-11, AAV-12, AAV-13, AAV-14, AAV-15 and AAV-16. AAV.rh8, AAV.rh10, AAV.rh20. AAV.rh39, AAV.Rh74, AAV.RHM4-1, AAV.hu37, AAV.hu32, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12. AAV.HSC13, AAV.HSC14, AAV.HSC15, or AAV.HSC16.

In some embodiments, rAAV particles comprise the capsid of Anc80 or Anc80L65, as described in Zinn et al., 2015, Cell Rep. 12(6): 1056-1068, which is incorporated by reference in its entirety. In certain embodiments, the rAAV particles comprise the capsid with one of the following amino acid insertions: LGETTRP (SEQ ID NO: 71) or LALGETTRP (SEQ ID NO: 72), as described in U.S. Pat. Nos. 9,193,956; 9,458,517; and 9,587,282 and US patent application publication no. 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise any AAV capsid disclosed in U.S. Pat. No. 9,585,971, such as AAV-PHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1. HSC2, HSC3, HSC4, HSC5, HSC6. HSC7, HSC8, HSC9, HSC10, HSC11, HSC12, HSC13, HSC14, HSC15, or HSC16, each of which is incorporated by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; U.S. Pat. No. 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, rAAV particles have a capsid protein at least 80% or more identical, e.g., 85%, 86%, 87%, 88%, 89%, 90%, 91%, 92%, 93%, 94% 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2 in '051 publication), WO 2005/033321 (see, e.g., SEQ ID NOs: 123 and 88 in '321 publication), WO 03/042397 (see, e.g., SEQ ID NOs: 2, 81, 85, and 97 in '397 publication), WO 2006/068888 (see, e.g., SEQ ID NOs: 1 and 3-6 in '888 publication), WO 2006/110689, (see, e.g., SEQ ID NOs: 5-38 in '689 publication) WO2009/104964 (see, e.g., SEQ ID NOs: 1-5, 7, 9, 20, 22, 24 and 31 in '964 publication), WO2010/127097 (see, e.g., SEQ ID NOs: 5-38 in '097 publication), and WO 2015/191508 (see, e.g., SEQ ID NOs: 80-294 in '508 publication), and U.S. Appl. Publ. No. 20150023924 (see, e.g., SEQ ID NOs: 1, 5-10 in '924 publication), the contents of each of which is herein incorporated by reference in its entirety. In some embodiments, rAAV particles have a capsid protein at least 80% or more identical, e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 in '051 publication), WO 2005/033321 (see, e.g., SEQ ID NOs: 123 and 88 in '321 publication), WO 03/042397 (see, e.g., SEQ ID NOs: 2, 81, 85, and 97 in '397 publication), WO 2006/068888 (see, e.g., SEQ ID NOs: 1 and 3-6 in '888 publication), WO 2006/110689 (see, e.g., SEQ ID NOs: 5-38 in '689 publication) WO2009/104964 (see, e.g., SEQ ID NOs: 1-5, 7, 9, 20, 22, 24 and 31 in '964 publication). WO 2010/127097 (see, e.g., SEQ ID NOs: 5-38 in '097 publication), and WO 2015/191508 (see, e.g., SEQ ID NOs: 80-294 ofin '508 publication), and U.S. Appl. Publ. No. 20150023924 (see, e.g., SEQ ID NOs: 1, 5-10 in '924 publication).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

The provided methods are suitable for used in the production of recombinant AAV encoding a transgene. In some embodiments, provided herein are rAAV viral vectors encoding an anti-VEGF Fab. In some embodiments, provided herein are rAAV8-based viral vectors encoding an anti-VEGF Fab. In more embodiments, provided herein are rAAV8-based viral vectors encoding ranibizumab. In some embodiments, provided herein are rAAV viral vectors encoding Iduronidase (IDUA). In some embodiments, provided herein are rAAV9-based viral vectors encoding IDUA. In some embodiments, provided herein are rAAV viral vectors encoding Iduronate 2-Sulfatase (IDS). In some embodiments, provided herein are rAAV9-based viral vectors encoding IDS. In some embodiments, provided herein are rAAV viral vectors encoding a low-density lipoprotein receptor (LDLR). In some embodiments, provided herein are rAAV8-based viral vectors encoding LDLR. In some embodiments, provided herein are rAAV viral vectors encoding tripeptidyl peptidase 1 (TPP1) protein. In some embodiments, provided herein are rAAV9-based viral vectors encoding TPP. In some embodiments, provided herein are rAAV viral vectors encoding anti-kallikrein (anti-pKal) antibody. In some embodiments, provided herein are rAAV8-based or rAAV9-based viral vectors encoding a pKal antibody Fab or full-length antibody.

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In some embodiments, the capsid protein is a chimeric of 2 or more AAV capsid proteins from AAV serotypes selected from AAV1, AAV1, AAV2, AAV3, AAV4, AAV5, AAV6, AAV7, AAV8, AAV9, AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV2.5, AAV2tYF, AAV3B, AAV.LK03, AAV.HSC1. AAV.HSC2, AAV.HSC3, AAV.HSC4, AAV.HSC5. AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty, D M, et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

“Self-complementary AAV” refers a plasmid or vector having an expression cassette in which a coding region carried by a recombinant AAV nucleic acid sequence has been designed to form an intra-molecular double-stranded DNA template. Unlike ssDNA genomes, the scAAV genome is not subject to host-cell DNA polymerase and does not require synthesis of a complementary strand. Upon infection, rather than waiting for cell mediated synthesis of the second strand, the two complementary halves of scAAV will associate to form one double stranded DNA (dsDNA) unit that is ready for immediate replication and transcription. See, e.g., McCarty, D M, et al, 2001, supra. Self-complementary AAVs are described in, e.g., U.S. Pat. Nos. 6,596,535; 7,125,717; and 7,456,683, each of which is incorporated herein by reference in its entirety. Genomes that are 2500 kb or less in size may benefit from packaging in self-complimentary AAV vectors.

Single-stranded AAV (ssAAV) vectors, wherein the coding sequence and complementary sequence of the transgene expression cassette are on separate strands, are packaged in separate viral capsids. For ssAAV, after transduction occurs and genome enters the nucleus, the single-to-double stranded conversion of the DNA undergoes inter-molecular annealing or second-strand synthesis. In certain embodiments, a single-stranded AAV (ssAAV) can be used. For self-complementary AAV (scAAV) vectors, both the coding and complementary sequence of the transgene expression cassette are present on each plus- and minus-strand genome. In contrast, a scAAV vector with half the size of the ssAAV genome has a mutation in the terminal resolution site (TRS) to form a vector genome with wild-type ITRs at both ends and mutated ITR at the center of symmetry. After uncoating in the target cell nucleus, this DNA structure can readily fold into transcriptionally active double-stranded form through intra-molecular annealing. In certain embodiments, a self-complementary vector, e.g., scAAV, can be used (see, e.g., Wu, 2007, supra; McCarty et al, 2001, supra). In some embodiments, the expression cassette comprises one ITR (5′-) mutated to form a self-complementary AAV (scAAV) genome or with two wild-type ITRs (5′- and 3′-) to form a single-stranded AAV (ssAAV) genome. Alternative ITR sequences are known in the art.

In some embodiments, rAAV particles comprise a capsid protein from an AAV capsid serotype selected from AAV-8 or AAV-9. In some embodiments, the rAAV particles have an AAV capsid serotype of AAV-1 or a derivative, modification, or pseudotype thereof. In some embodiments, the rAAV particles have an AAV capsid serotype of AAV-4 or a derivative, modification, or pseudotype thereof. In some embodiments, the rAAV particles have an AAV capsid serotype of AAV-5 or a derivative, modification, or pseudotype thereof. In some embodiments, the rAAV particles have an AAV capsid serotype of AAV-8 or a derivative, modification, or pseudotype thereof. In some embodiments, the rAAV particles have an AAV capsid serotype of AAV-9 or a derivative, modification, or pseudotype thereof.

In some embodiments, rAAV particles comprise a capsid protein that is a derivative, modification, or pseudotype of AAV-8 or AAV-9 capsid protein. In some embodiments, rAAV particles comprise a capsid protein that has an AAV-8 capsid protein at least 80% or more identical. e.g., 85%, 86%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV-8 capsid protein.

In some embodiments, rAAV particles comprise a capsid protein that is a derivative, modification, or pseudotype of AAV-9 capsid protein. In some embodiments, rAAV particles in the clarified feed comprise a capsid protein that has an AAV-8 capsid protein at least 80% or more identical, e.g., 85%, 86%, 87%, 88%, 89%, 90%, 91%, 92%, 93%, 94%, 95%, 96%, 97%, 98%, 99%. 99.5%, etc., i.e. up to 100% identical, to the VP1, VP2 and/or VP3 sequence of AAV-9 capsid protein.

In additional embodiments, rAAV particles comprise a mosaic capsid. Mosaic AAV particles are composed of a mixture of viral capsid proteins from different serotypes of AAV. In some embodiments, rAAV particles comprise a mosaic capsid containing capsid proteins of a serotype selected from AAV1, AAV2, AAV3, AAV4, AAV5, AAV6, AAV7, AAV8, AAV9. AAV10, AAV-11, AAV-12, AAV-13, AAV-14, AAV-15 and AAV-16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV2.5, AAV2tYF, AAV3B, AAV.LK03, AAV.HSC1, AAV.HSC2. AAV.HSC3. AAV.HSC4. AAV.HSC5. AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, rAAV particles comprise a mosaic capsid containing capsid proteins of a serotype selected from AAV-1, AAV-2, AAV-5, AAV-6, AAV-7, AAV-8, AAV-9, AAV-10, AAVrh.8, and AAVrh.10. In additional embodiments, rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV-1, AAV-3, AAV-4, AAV-5, AAV-6, AAV-7, AAV-8, AAV-9, AAV-10 AAV-11, AAV-12. AAV-13, AAV-14. AAV-15 and AAV-16). In additional embodiments, rAAV particles comprise a pseudotyped rAAV particle comprised of a capsid protein of an AAV serotype selected from AAV1, AAV2, AAV3, AAV4, AAV5, AAV6, AAV7, AAV8. AAV9, AAV10, AAV-11, AAV-12, AAV-13, AAV-14, AAV-15 and AAV-16, AAV.rh8, AAV.rh10. AAV.rh20, AAV.rh39, AAV.Rh74, AAV.RHM4-1, AAV.hu37, AAV.hu32, AAV.Anc80, AAV.Anc80L65, AAV.7m8, AAV.PHP.B. AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8. AAV.HSC9, AAV.HSC10, AAV.HSC11, AAV.HSC12, AAV.HSC13, AAV.HSC14, AAV.HSC15, and AAV.HSC16. In additional embodiments, rAAV particles comprise a pseudotyped rAAV particle containing AAV-8 capsid protein. In additional embodiments, rAAV particles comprise a pseudotyped rAAV particle is comprised of AAV-9 capsid protein. In some embodiments, the pseudotyped rAAV8 or rAAV9 particles are rAAV2/8 or rAAV2/9 pseudotyped particles. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In further embodiments, the capsid protein is a chimeric of 2 or more AAV capsid proteins from AAV serotypes selected from AAV1, AAV2, rAAV3. AAV4, AAV5, AAV6, AAV7, AAV8, AAV9, AAV10, AAV11, AAV12, AAV 13, AAV14. AAV 15 and AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV2.5, AAV2tYF, AAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12. AAV.HSC13, AAV.HSC14, AAV.HSC15, and AAV.HSC16. In further embodiments, the capsid protein is a chimeric of 2 or more AAV capsid proteins from AAV serotypes selected from AAV1, AAV2, AAV5, AAV6, AAV7, AAV8, AAV9, AAV10, AAVrh.8, and AAVrh.10.

In some embodiments, the rAAV particles comprise an AAV capsid protein chimeric of AAV-8 capsid protein and one or more AAV capsid proteins from an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8. AAV.PHP.B, AAV2.5, AAV2tYF, AAV3B, AAV.LK03. AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV-8 capsid protein and one or more AAV capsid proteins from an AAV serotype selected from AAV1, AAV2, AAV5, AAV6, AAV7, AAV9, AAV10, AAVrh.8, and AAVrh.10.

In some embodiments, the rAAV particles comprise an AAV capsid protein chimeric of AAV-9 capsid protein the capsid protein of one or more AAV capsid serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, the rAAV particles comprise an AAV capsid protein chimeric of AAV-9 capsid protein the capsid protein of one or more AAV capsid serotypes selected from AAV1, AAV2, AAV3, AAV4, AAV5, AA6, AAV7, AAV8, AAV9, AAVrh.8, and AAVrh.10.

In embodiments, rAAV comprising a recombinant expression cassette comprising a composite nucleic acid regulatory element comprising or consisting of Mus022.CK and Mus035.CK, have reduced activity in cardiac muscle compared to CK7 and Spc5-12 promoters, despite having similar skeletal muscle-specific activity, rAAV comprising a recombinant expression cassette comprising a composite nucleic acid regulatory element comprising or consisting of Mus022.CK and Mus035.CK exhibited significantly less RNA expression in heart providing a gene expression profile that is advantageous for linking operably the Mus022.CK promoter to therapeutic genes delivered preferentially to skeletal muscle relative to cardiac tissue.

Methods of Making rAAV Vectors

Another aspect of the present invention involves making molecules disclosed herein. In some embodiments, a molecule according to the invention is made by providing a nucleotide comprising the nucleic acid sequence encoding an AAV capsid protein; and using a packaging cell system to prepare corresponding rAAV particles with capsid coats made up of the capsid protein. In some embodiments, the nucleic acid sequence encodes a sequence having at least 60%, 70%, 80%, 85%, 90%, or 95%, 96%, 97%, 98%, 99% or 99.9%, identity to the sequence of a capsid protein molecule described herein, and retains (or substantially retains) biological function of the capsid protein and the inserted peptide from a heterologous protein or domain thereof. In some embodiments, the nucleic acid encodes a sequence having at least 60%, 70%, 80%, 85%, 90%, or 95%, 96%, 97%, 98%, 99% or 99.9%, identity to a particular sequence of the AAV capsid protein, while retaining (or substantially retaining) biological function of the AAV capsid protein.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In certain embodiments, the cap and rep genes are provided by a packaging cell and not present in the viral genome.

In some embodiments, the nucleic acid encoding the capsid protein is cloned into an AAV Rep-Cap helper plasmid in place of the existing capsid gene. When introduced together into host cells, this plasmid helps package an rAAV genome into the capsid protein as the capsid coat. Packaging cells can be any cell type possessing the genes necessary to promote AAV genome replication, capsid assembly, and packaging. Nonlimiting examples include 293 cells or derivatives thereof, HELA cells, or insect cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In preferred embodiments, the rAAVs provide transgene delivery vectors that can be used in therapeutic and prophylactic applications, as discussed in more detail below. The rAAV vector also includes the regulatory control elements discussed supra to influence the expression of the RNA and/or protein products encoded by nucleic acids (transgenes) within target cells of the subject.

Provided in particular embodiments are AAV vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the AAV capsid protein.

The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

The rAAV vector for delivering the transgene to target tissues, cells, or organs, may also have a tropism for that particular target tissue, cell, or organ, e.g. liver and/or muscle, in conjunction with the use of tissue-specific promoters as described herein. The construct can further include additional expression control elements such as introns that enhance expression of the transgene (e.g., introns such as the chicken β-actin intron, minute virus of mice (MVM) intron, human factor IX intron (e.g., FIX truncated intron 1). β-globin splice donor/immunoglobulin heavy chain splice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit D3-globin polyA signal, human growth hormone (hGH) polyA signal, SV40 late polyA signal, synthetic polyA (SPA) signal, and bovine growth hormone (bGH) polyA signal. See, e.g., Powell and Rivera-Soto, 2015, Discov. Med., 19(102):49-57.

In certain embodiments, nucleic acids sequences disclosed herein may be codon-optimized, for example, via any codon-optimization technique known to one of skill in the art (see, e.g., review by Quax et al., 2015, Mol Cell 59:149-161).

In one embodiment, the constructs described herein comprise the following components: (1) AAV inverted terminal repeats (ITRs) that flank the expression cassette; (2) control elements, which include a) one or more regulatory elements at least including one or more of the enhancers of any one of SEQ ID NOs: 10-13, alone or in combination with one or more promoters, b) a poly A signal, and c) optionally an intron; (3) transgene providing (e.g., coding for) one or more RNA or protein products of interest. In another embodiment, the constructs described herein comprise the following components: (1) AAV inverted terminal repeats (ITRs) that flank the expression cassette; (2) control elements, which include a) one or more regulatory elements at least including one or more of the enhancers of any one of SEQ ID NO:s 10-13, alone or in combination with one or more a CK promoter, b) a poly A signal, and c) optionally an intron; (3) transgene providing (e.g., coding for) one or more RNA or protein products of interest. In yet another embodiment, the constructs described herein comprise the following components: (1) AAV inverted terminal repeats (ITRs) that flank the expression cassette; (2) control elements, which include a) one or more regulatory elements at least including a Mus022 enhancers (SEQ ID NO: 10), in combination with a CK promoter (SEQ ID NO: 8), b) a poly A signal, and c) optionally an intron; (3) transgene providing (e.g., coding for) one or more RNA or protein products of interest, such as those in Tables 4A, 4B or 4C.

In a certain embodiment, the constructs described herein comprise the following components: (1) AAV inverted terminal repeats (ITRs) that flank the expression cassette; (2) control elements, which include a) one or more regulatory elements at least including one or more of the promoters of any one of SEQ ID NO:s 14-26, alone or in combination with one or more enhancers, b) a poly A signal, and c) optionally an intron; (3) transgene providing (e.g., coding for) one or more RNA or protein products of interest. In another embodiment, the constructs described herein comprise the following components: (1) AAV inverted terminal repeats (ITRs) that flank the expression cassette; (2) control elements, which include a) one or more regulatory elements at least including one or more of the ACTA core promoters of any one of SEQ ID NOs: 14-26, alone or in combination with one or more enhancers of any one of SEQ ID NOs: 4-7, b) a poly A signal, and c) optionally an intron; (3) transgene providing (e.g., coding for) one or more RNA or protein products of interest, such as those in Tables 4A, 4B or 4C.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCL sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or muscle or other cell types may be used, for example, but not limited, to HuH-7, HEK293, fibrosarcoma HT-1080, HKB-11, C2C12 myoblasts, and CAP cells. Once expressed, characteristics of the expressed product (transgene product) can also be determined, including serum half-life, functional activity of the protein (e.g. enzymatic activity or binding to a target), determination of the glycosylation and tyrosine sulfation patterns, and other assays known in the art for determining protein characteristics.

Provided are methods of manufacturing a recombinant AAV comprising culturing a host cell capable of producing a recombinant AAV described herein under conditions appropriate for production of the recombinant AAV comprising an artificial genome with an expression cassette comprising a synthetic promoter operably linked to a transgene. In particular, the method provides (1) culturing a host cell containing (i) an artificial genome comprising AAV ITRs flanking a recombinant cis expression cassette which comprises a nucleic acid regulatory element comprising a composite nucleic acid regulatory element as disclosed herein operably linked to a transgene; (ii) a trans expression cassette lacking AAV ITRs which encodes an AAV rep and an AAV capsid protein operably linked to expression control elements that drive expression of the AAV rep and the AAV capsid protein in the host cell in culture and supply the AAV rep and the AAV capsid protein in trans; and (iii) sufficient adenovirus helper functions to permit replication and packaging of the artificial genome by the AAV capsid protein; and (2) recovering recombinant AAV encapsidating the artificial genome from the cell culture. Also provided are host cells containing (i) an artificial genome comprising AAV ITRs flanking a recombinant cis expression cassette which comprises a composite nucleic acid regulatory element disclosed herein operably linked to a transgene; (ii) a trans expression cassette lacking AAV ITRs which encodes an AAV rep and an AAV capsid protein operably linked to expression control elements that drive expression of the AAV rep and the AAV capsid protein in the host cell in culture and supply the AAV rep and the AAV capsid protein in trans; and, optionally, (iii) sufficient adenovirus helper functions to permit replication and packaging of the artificial genome by the AAV capsid protein. In particular embodiments, the composite nucleic acid regulatory element is Mus022.CK or SEQ ID NO: 9. In certain embodiments, the artificial genome comprises a transgene encoding one of the therapeutics listed in Tables 4A, 4B and 4C.

### 5.4. Therapeutic and Prophylactic Uses

Another aspect relates to therapies which involve administering a transgene via a rAAV vector according to the invention to a subject in need thereof, for delaying, preventing, treating, and/or managing a disease or disorder, and/or ameliorating one or more symptoms associated therewith. A subject in need thereof includes a subject suffering from the disease or disorder, or a subject pre-disposed thereto, e.g., a subject at risk of developing or having a recurrence of the disease or disorder. Generally, a rAAV carrying a particular transgene will find use with respect to a given disease or disorder in a subject where the subject's native gene, corresponding to the transgene, is defective in providing the correct gene product, or correct amounts of the gene product. The transgene then can provide a copy of a gene that is defective in the subject.

Generally, the transgene comprises cDNA that restores protein function to a subject having a genetic mutation(s) in the corresponding native gene. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

In some aspects, the therapeutic encoded by one or more of the disclosed transgenes can by a microdystrophin. Microdystrophins include those having the amino acid sequence of microdystrophins that consist of dystrophin domains arranged amino-terminus to the carboxy terminus; ABD-H1-R1-R2-R3-H3-R24-H4-CR-CT, wherein ABD is an actin-binding domain of dystrophin, H1 is a hinge 1 region of dystrophin, R1 is a spectrin 1 region of dystrophin. R2 is a spectrin 2 region of dystrophin, R3 is a spectrin 3 region of dystrophin, H3 is a hinge 3 region of dystrophin, R24 is a spectrin 24 region of dystrophin, H4 is a hinge 4 region of dystrophin, CR is a cysteine-rich region of dystrophin and CT is the C terminal domain (and comprises at least the portion of the CT domain containing the al-syntrophin binding site), such as the microdystrophin of RGX-202. The amino acid sequences for the components of dystrophin used to form a microdystrophin or a mini-dystrophin, are described in the full length human DMD protein UniProtDB-11532, which is incorporated by reference herein. Additional embodiments are disclosed in International Application PCT/US2020/062484, filed Nov. 27, 2020, which is hereby incorporated by reference in its entirety.

Provided are methods of treating human subjects for any muscular dystrophy disease that can be treated by providing a functional dystrophin. In some aspects, the functional dystrophin is one or more of the microdystrophins disclosed herein. DMD is the most common muscular dystrophy disease, but other diseases can be treated such as, but not limited to, Becker muscular dystrophy (BMD), myotonic muscular dystrophy (Steinert's disease), Facioscapulohumeral disease (FSHD), limb-girdle muscular dystrophy, X-linked dilated cardiomyopathy, or oculopharyngeal muscular dystrophy.

Tables 4A, 4B and 4C below provides a list of transgenes that may be used in any of the recombinant expression cassettes described herein, preferably to treat or prevent the disease with which it is associated, also listed in Tables 4A-4B. Table 4A includes, but is not limited to, a number of transgenes when delivered to a patient via muscle secretion, via blood circulation, and/or via the CNS, at a level determined to be efficacious are known to repair a patient's muscle cells or muscle activity. Several transgenes are muscle-derived proteins. As described herein, the AAV vector may be engineered as described herein to target the appropriate tissue for delivery of the transgene to effect the therapeutic or prophylactic use. The appropriate AAV serotype may be chosen to engineer to optimize the tissue tropism and transduction of the vector.

TABLE 4A

| Disease | Transgene |
|---|---|
| Spinal Muscular Atrophy (SMA) | SMN |
| Duchenne Muscular Dystrophy | Mini-/Micro- |
| | Dystrophin Gene |
| Limb Girdle Muscular Dystrophy Type 2C\|Gamma- | human-alpha- |
| sarcoglycanopathy | sarcoglycan |
| Limb Girdle Muscular Dystrophy Type 2C\|Gamma- | gamma-sarcoglycan |
| sarcoglycanopathy | |
| Becker Muscular Dystrophy and Sporadic | huFollistatin344 |
| Inclusion Body Myositis | |
| Duchenne Muscular Dystrophy | GALGT2 |
| Limb-Girdle Muscular Dystrophy, Type 2E | hSGCB |
| Limb-Girdle Muscular Dystrophy (various types) | hAUF1 |
| Limb Girdle Muscular Dystrophy Type 2B | dysferlin |
| Limb Girdle Muscular Dystrophy Type 2i | FKRP |

TABLE 4B

| ANTIGENS | | TRANSGENE/ANTIBODIES | INDICATIONS |
|---|---|---|---|
| Repulsive guidance molecule-A | | elezanumab | multiple sclerosis |
| Complement Component 5 | | ravulizumab | Myasthenia Gravis |
| Connective tissue growth factor | | pamrevlumab | fibrotic diseases, e.g. |
| (CTGF) | | | diabetic nephropathy, |
| | | | liver fibrosis, idiopathic |
| | | | pulmonary fibrosis |
| Integrin beta 7 | | etrolizumab | ulcerative colitis, Crohn's |
| | | | disease |
| Sclerostin | | romosozumab (EVENITY ®) | Osteoporosis, abnormal |
| | | | bone loss or weakness |
| Interleukin receptor 6 (IL6R) or | | Satralizumab | Adverse immune |
| Interleukin 6 (IL6) | | Sarilumab | responses (e.g. cytokine |
| | | Tocilizumab | storm, CAR-T therapy) |
| | | siltuximab, | |
| | | clazakizumab | |
| | | sirukumab | |
| | | olokizumab | |
| | | gerilimzumab | |
| Immunoglobin E (IgE) | | omolizumab | Asthma, COPD, |
| | | | eosinophilic asthma, |
| | | | chronic idiopathic |
| | | | urticaria |
| Thymic stromal lymphopoietin | | tezelipumab | Asthma, COPD |
| (TSLP) | | | |
| Interleukin 5 (IL5) | | benralizumab | Asthma, COPD |
| Interleukin 5 receptor (IL5R) | | reslizumab | Asthma, COPD, |
| | | | eosinophilic asthma |
| Interleukin 13 (IL13) | | tralokinumab | Atopic dermatitis |
| Interleukin 31 recptor alpha | | nemolizumab | Atopic dermatitis |
| (IL31RA) | | | |
| IL17A | | Ixekizumab | Plaque psoriasis, |
| | | secukinumab | psoriatic arthritis, |
| | | | ankylosing sponylitis |
| Interleukins or | IL-17A | ixekizumab (TALTZ ®) | Plaque psoriasis, |
| interleukin | | secukinumab | psoriatic arthritis, |
| receptors | | (COSENTYX ®) | ankylosing sponylitis |
| | IL-5 | mepolizumab (NUCALA ®) | Asthma |
| | IL-12/IL-23 | ustekinumab (STELARA ®) | Psoriasis & Crohn's |
| | | | disease |
| | IL-4R | dupilumab | Atopic dermatitis |
| Integrin | | vedolizumab (ENTYVIO ®) | Ulcerative colitis & |
| | | | Crohn's disease |
| | | Natalizumab (anti-integrin | Multiple sclerosis & |
| | | alpha 4) | Crohn's disease |
| Cardiovascular | PCSK9 | alirocumab (PRALUENT ®) | HeFH & HoFH |
| Targets | | evolucomab (REPATHA ®) | |
| | ANGPTL3 | evinacumab | HoFH & severe forms of |
| | | | dyslipidema |
| | Proinflammatory/ | E06-scFv | Cardiovascular diseases |
| | proatherogenic | | such as atherosclerosis |
| | phospholipids | | |
| RANKL | | denosumab (XGEVA ® and | Osteoporosis, increasing |
| | | PROLIA ®) | bone mass in breast and |
| | | | prostate cancer patients, |
| | | | & preventing skeletal- |
| | | | related events due to |
| | | | bone metastasis |
| PD-1, or PD-L1 or PD-L2 | | nivolumab (OPDIVO ®) | Metastatic melanoma, |
| | | pembrolizumab | lymphoma, non-small |
| | | (KEYTRUDA ®) | cell lung carcinoma |
| BLyS (B-lymphocyte stimulator. also | | belimumab (BENLYSTA ®) | Systemic lupus |
| known as B-cell activating factor | | | erythromatosis |
| (BAFF)) | | | |
| TNF-alpha | | adalimumab (HUMIRA ®) | Rheumatoid arthritis, |
| | | and | psoriatic arthritis, |
| | | infliximab (REMICADE ®) | askylosing spondylitis, |
| | | | Crohn's disease, plaque |
| | | | psoriasis, ulcerative |
| | | | colitis |
| Plasma Protein | C5, C5a | eculizumab (SOLIRIS ®) | Paroxysmal nocturnal |
| targets | | | hemoglobinuria, atypical |
| | | | hemolytic uremic |
| | | | syndrome, complement- |
| | | | mediated thrombotic |
| | | | microangiopathy |
| | Plasma kallikrein | lanadelumab | Hereditary angioedema |
| | | | (HAE) |

In some embodiments, provided are recombinant expression cassettes comprising a minidystrophin or microdystrophin transgene. In embodiments, the microdystrophin comprises dystrophin domains arranged amino-terminus to the carboxy terminus: ABD-H1-R1-R2-R3-H3-R24-H4-CR-CT, w herein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, H4 is a hinge 4 region of dystrophin, CR is a cysteine-rich region of dystrophin and CT is the C terminal domain, or a portion thereof.

The present disclosure contemplates variants of microdystrophin so long as the therapeutic efficacy of microdystrophin comprising such variants is substantially maintained. Functional activity includes (1) binding to one of, a combination of, or all of actin, β-dystroglycan, α1-syntrophin, α-dystrobrevin, and nNOS; (2) improved muscle function in an animal model (for example, in the mdx mouse model) or in human subjects; and/or (3) cardioprotective or improvement in cardiac muscle function in animal models or human patients.

Table 4C provides the amino acid sequences of the microdystrophin embodiments in accordance with the present disclosure. In certain embodiments, the microdystrophin has an amino acid sequence of SEQ ID NOs: 73 (DYS1), 74 (DYS3), or 75 (DYS5). In other embodiments, the microdystrophin has an amino acid sequence of SEQ ID NO: 76 (human MD1 (R4-R23/ACT), SEQ ID NO: 77 (microdystrophin), SEQ ID NO: 78 (Dys3978), SEQ ID NO: 79 (MD3) or SEQ ID NO: 80 (MD4). It is also contemplated that other embodiments are substituted variants of microdystrophins as defined by SEQ ID NOs: 73 (DYS1), 74 (DYS3), or 75 (DYS5).

In embodiments, microdystrophin may have at least 60%, at least 70%, at least 80%, at least 85%, at least 90%, at least 95%, at least 96%, at least 97%, at least 98%, or at least 99% sequence identity to the amino acid sequence of SEQ ID NOs: 73, 74 or 75 and maintain functional microdystrophin activity, as determined, for example, by one or more of the in vitro assays or in vivo assays in animal models disclosed in, for example, WO 2021/108755.

TABLE 4C
Amino acid sequences of RGX-DYS and Microdystrophin proteins

| | SEQ | |
|---|---|---|
| | ID | |
| Structure | NO: | Amino Acid Sequence |
| DYS1 | 73 | MLWWEEVEDCYEREDVQKKTFTKWVNAQFSKFGKQHIENLFSDLQDGRRL |
| | | LDLLEGLTGQKLPKEKGSTRVHALNNVNKALRVLQNNNVDLVNIGSTDIV |
| | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSL |
| | | QKLAVLKADLEKKKQSMGKLYSLKQDLLSTLKNKSVTQKTEAWLDNFARC |
| | | WDNLVQKLEKSTAQISQQPDLAPGLTTIGASPTQTVTLVTQPVVTKETAI |
| | | SKLEMPSSLMLEVPTLERLQELQEATDELDLKLRQAEVIKGSWQPVGDLL |
| | | IDSLQDHLEKVKALRGEIAPLKENVSHVNDLARQLTTLGIQLSPYNLSTL |
| | | EDLNTRWKLLQVAVEDRVRQLHEAHRDFGPASQHELSTSVQGPWERAISP |
| | | NKVPYYINHETQTTCWDHPKMTELYQSLADLNNVRFSAYRTAMKLRRLQK |
| | | ALCLDLLSLSAACDALDQHNLKQNDQPMDILQIINCLTTIYDRLEQEHNN |
| | | LVNVPLCVDMCLNWLLNVYDTGRTGRIRVLSFKTGIISLCKAHLEDKYRY |
| | | LFKQVASSTGFCDQRRLGLLLHDSIQIPRQLGEVASFGGSNIEPSVRSCF |
| | | QFANNKPEIEAALFLDWMRLEPQSMVWLPVLHRVAAAETAKHQAKCNICK |
| | | ECPIIGFRYRSLKHFNYDICQSCFFSGRVAKGHKMHYPMVEYCTPTTSGE |
| | | DVRDFAKVLKNKFRTKRYFAKHPRMGYLPVQTVLEGDNMETPVTLINFWP |
| | | VDSAPASSPQLSHDDTHSRIEHYASRLAEMENSNGSYLNDSISPNESIDD |
| | | EHLLIQHYCQSLNQDSPLSQPRSPAQILISLESEERGELERILADLEEEN |
| | | RNLQAEYDRLKQQHEHKGLSPLPSPPEMMPTSPQSPR |
| DYS3 | 74 | MLWWEEVEDCYEREDVQKKTFTKWVNAQFSKFGKQHIENLESDLQDGRRLL |
| | | DLLEGLTGQKLPKEKGSTRVHALNNVNKALRVLQNNNVDLVNIGSTDIVDG |
| | | NHKLTLGLIWNIILHWQVKNVMKNIMAGLQQTNSEKILLSWVRQSTRNYPQ |
| | | VNVINFTTSWSDGLALNALIHSHRPDLFDWNSVVCQQSATQRLEHAFNIAR |
| | | YQLGIEKLLDPEDVDTTYPDKKSILMYITSLFQVLPQQVSIEAIQEVEMLP |
| | | RPPKVTKEEHFQLHHQMHYSQQITVSLAQGYERTSSPKPRFKSYAYTQAAY |
| | | VTTSDPTRSPFPSQHLEAPEDKSFGSSLMESEVNLDRYQTALEEVLSWLLS |
| | | AEDTLQAQGEISNDVEVVKDQFHTHEGYMMDLTAHQGRVGNILQLGSKLIG |
| | | TGKLSEDEETEVQEQMNLLNSRWECLRVASMEKQSNLHRVLMDLQNQKLKE |
| | | LNDWLTKTEERTRKMEEEPLGPDLEDLKRQVQQHKVLQEDLEQEQVRVNSL |
| | | THMVVVVDESSGDHATAALEEQLKVLGDRWANICRWTEDRWVLLQDILLKW |
| | | QRLTEEQCLFSAWLSEKEDAVNKIHTTGFKDQNEMLSSLQKLAVLKADLEK |
| | | KKQSMGKLYSLKQDLLSTLKNKSVTQKTEAWLDNFARCWDNLVQKLEKSTA |
| | | QISQQPDLAPGLTTIGASPTQTVTLVTQPVVTKETAISKLEMPSSLMLEVP |
| | | TLERLQELQEATDELDLKLRQAEVIKGSWQPVGDLLIDSLQDHLEKVKALR |
| | | GEIAPLKENVSHVNDLARQLTTLGIQLSPYNLSTLEDLNTRWKLLQVAVED |
| | | RVRQLHEAHRDFGPASQHELSTSVQGPWERAISPNKVPYYINHETQTTCWD |
| | | HPKMTELYQSLADLNNVRESAYRTAMKLRRLQKALCLDLLSLSAACDALDQ |
| | | HNLKQNDQPMDILQIINCLTTIYDRLEQEHNNLVNVPLCVDMCLNWLLNVY |
| | | DTGRTGRIRVLSFKTGIISLCKAHLEDKYRYLFKQVASSTGFCDQRRLGLL |
| | | LHDSIQIPRQLGEVASFGGSNIEPSVRSCFQFANNKPEIEAALFLDWMRLE |
| | | PQSMVWLPVLHRVAAAETAKHQAKCNICKECPIIGFRYRSLKHENYDICQS |
| | | CFFSGRVAKGHKMHYPMVEYCTPTTSGEDVRDFAKVLKNKFRTKRYFAKHP |
| | | RMGYLPVQTVLEGDNMET |
| DYS5 | 75 | MLWWEEVEDCYEREDVQKKTFTKWVNAQFSKFGKQHIENLFSDLQDGRRLL |
| | | DLLEGLTGQKLPKEKGSTRVHALNNVNKALRVLQNNNVDLVNIGSTDIVDG |
| | | NHKLTLGLIWNIILHWQVKNVMKNIMAGLQQTNSEKILLSWVRQSTRNYPQ |
| | | VNVINFTTSWSDGLALNALIHSHRPDLFDWNSVVCQQSATQRLEHAFNIAR |
| | | YQLGIEKLLDPEDVDTTYPDKKSILMYITSLFQVLPQQVSIEAIQEVEMLP |
| | | RPPKVTKEEHFQLHHQMHYSQQITVSLAQGYERTSSPKPRFKSYAYTQAAY |
| | | VTTSDPTRSPFPSQHLEAPEDKSFGSSLMESEVNLDRYQTALEEVLSWLLS |
| | | AEDTLQAQGEISNDVEVVKDQFHTHEGYMMDLTAHQGRVGNILQLGSKLIG |
| | | TGKLSEDEETEVQEQMNLLNSRWECLRVASMEKQSNLHRVLMDLQNQKLKE |
| | | LNDWLTKTEERTRKMEEEPLGPDLEDLKRQVQQHKVLQEDLEQEQVRVNSL |
| | | THMVVVVDESSGDHATAALEEQLKVLGDRWANICRWTEDRWVLLQDILLKW |
| | | QRLTEEQCLFSAWLSEKEDAVNKIHTTGFKDQNEMLSSLQKLAVLKADLEK |
| | | KKQSMGKLYSLKQDLLSTLKNKSVTQKTEAWLDNFARCWDNLVQKLEKSTA |
| | | QISQQPDLAPGLTTIGASPTQTVTLVTQPVVTKETAISKLEMPSSLMLEVP |
| | | TLERLQELQEATDELDLKLRQAEVIKGSWQPVGDLLIDSLQDHLEKVKALR |
| | | GEIAPLKENVSHVNDLARQLTTLGIQLSPYNLSTLEDLNTRWKLLQVAVED |
| | | RVRQLHEAHRDFGPASQHELSTSVQGPWERAISPNKVPYYINHETQTTCWD |
| | | HPKMTELYQSLADLNNVRESAYRTAMKLRRLQKALCLDLLSLSAACDALDQ |
| | | HNLKQNDQPMDILQIINCLTTIYDRLEQEHNNLVNVPLCVDMCLNWLLNVY |
| | | DTGRTGRIRVLSFKTGIISLCKAHLEDKYRYLFKQVASSTGFCDQRRLGLL |
| | | LHDSIQIPRQLGEVASFGGSNIEPSVRSCFQFANNKPEIEAALFLDWMRLE |
| | | PQSMVWLPVLHRVAAAETAKHQAKCNICKECPIIGFRYRSLKHENYDICQS |
| | | CFFSGRVAKGHKMHYPMVEYCTPTTSGEDVRDFAKVLKNKFRTKRYFAKHP |
| | | RMGYLPVQTVLEGDNMETPVTLINFWPVDSAPASSPQLSHDDTHSRIEHYA |
| | | SRLAEMENSNGSYLNDSISPNESIDDEHLLIQHYCQSLNQDSPLSQPRSPA |
| | | QILISLES |
| human MD1 | 76 | MLWWEEVEDCYEREDVQKKTFTKWVNAQFSKFGKQHIENLFSDLQDGRRLL |
| (R4- | | DLLEGLTGQKLPKEKGSTRVHALNNVNKALRVLQNNNVDLVNIGSTDIVDG |
| R23/ΔCT) | | NHKLTLGLIWNIILHWQVKNVMKNIMAGLQQTNSEKILLSWVRQSTRNYPQ |
| | | VNVINFTTSWSDGLALNALIHSHRPDLFDWNSVVCQQSATQRLEHAFNIAR |
| | | YQLGIEKLLDPEDVDTTYPDKKSILMYITSLFQVLPQQVSIEAIQEVEMLP |
| | | RPPKVTKEEHFQLHHQMHYSQQITVSLAQGYERTSSPKPRFKSYAYTQAAY |
| | | VTTSDPTRSPFPSQHLEAPEDKSFGSSLMESEVNLDRYQTALEEVLSWLLS |
| | | AEDTLQAQGEISNDVEVVKDQFHTHEGYMMDLTAHQGRVGNILQLGSKLIG |
| | | TGKLSEDEETEVQEQMNLLNSRWECLRVASMEKQSNLHRVLMDLQNQKLKE |
| | | LNDWLTKTEERTRKMEEEPLGPDLEDLKRQVQQHKVLQEDLEQEQVRVNSL |
| | | THMVVVVDESSGDHATAALEEQLKVLGDRWANICRWTEDRWVLLQDILLKW |
| | | QRLTEEQCLFSAWLSEKEDAVNKIHTTGFKDQNEMLSSLQKLAVLKADLEK |
| | | KKQSMGKLYSLKQDLLSTLKNKSVTQKTEAWLDNFARCWDNLVQKLEKSTA |
| | | QISQAVTTTQPSLTQTTVMETVTTVTTREQILVKHAQEELPPPPPQKKRQI |
| | | TVDTLERLQELQEATDELDLKLRQAEVIKGSWQPVGDLLIDSLQDHLEKVK |
| | | ALRGEIAPLKENVSHVNDLARQLTTLGIQLSPYNLSTLEDLNTRWKLLQVA |
| | | VEDRVRQLHEAHRDFGPASQHELSTSVQGPWERAISPNKVPYYINHETQTT |
| | | CWDHPKMTELYQSLADLNNVRFSAYRTAMKLRRLQKALCLDLLSLSAACDA |
| | | LDQHNLKQNDQPMDILQIINCLTTIYDRLEQEHNNLVNVPLCVDMCLNWLL |
| | | NVYDTGRTGRIRVLSFKTGIISLCKAHLEDKYRYLFKQVASSTGFCDQRRL |
| | | GLLLHDSIQIPRQLGEVASFGGSNIEPSVRSCFQFANNKPEIEAALFLDWM |
| | | RLEPQSMVWLPVLHRVAAAETAKHQAKCNICKECPIIGFRYRSLKHENYDI |
| | | CQSCFFSGRVAKGHKMHYPMVEYCTPTTSGEDVRDFAKVLKNKFRTKRYFA |
| | | KHPRMGYLPVQTVLEGDNMETDTM |
| Human | 77 | MLWWEEVEDCYEREDVQKKTFTKWVNAQFSKFGKQHIENLFSDLQDGRRLL |
| micro- | | DLLEGLTGQKLPKEKGSTRVHALNNVNKALRVLQNNNVDLVNIGSTDIVDG |
| dystrophin | | NHKLTLGLIWNIILHWQVKNVMKNIMAGLQQTNSEKILLSWVRQSTRNYPQ |
| | | VNVINFTTSWSDGLALNALIHSHRPDLFDWNSVVCQQSATQRLEHAFNIAR |
| | | YQLGIEKLLDPEDVDTTYPDKKSILMYITSLFQVLPQQVSIEAIQEVEMLP |
| | | RPPKVTKEEHFQLHHQMHYSQQITVSLAQGYERTSSPKPRFKSYAYTQAAY |
| | | VTTSDPTRSPFPSQHLEAPEDKSFGSSLMESEVNLDRYQTALEEVLSWLLS |
| | | AEDTLQAQGEISNDVEVVKDQFHTHEGYMMDLTAHQGRVGNILQLGSKLIG |
| | | TGKLSEDEETEVQEQMNLLNSRWECLRVASMEKQSNLHRVLMDLQNQKLKE |
| | | LNDWLTKTEERTRKMEEEPLGPDLEDLKRQVQQHKVLQEDLEQEQVRVNSL |
| | | THMVVVVDESSGDHATAALEEQLKVLGDRWANICRWTEDRWVLLQDILLKW |
| | | QRLTEEQCLFSAWLSEKEDAVNKIHTTGFKDQNEMLSSLQKLAVLKADLEK |
| | | KKQSMGKLYSLKQDLLSTLKNKSVTQKTEAWLDNFARCWDNLVQKLEKSTA |
| | | QISQAVTTTQPSLTQTTVMETVTTVTTREQILVKHAQEELPPPPPQKKRTL |
| | | ERLQELQEATDELDLKLRQAEVIKGSWQPVGDLLIDSLQDHLEKVKALRGE |
| | | IAPLKENVSHVNDLARQLTTLGIQLSPYNLSTLEDLNTRWKLLQVAVEDRV |
| | | RQLHEAHRDFGPASQHFLSTSVQGPWERAISPNKVPYYINHETQTTCWDHP |
| | | KMTELYQSLADLNNVRESAYRTAMKLRRLQKALCLDLLSLSAACDALDQHN |
| | | LKQNDQPMDILQIINCLTTIYDRLEQEHNNLVNVPLCVDMCLNWLLNVYDT |
| | | GRTGRIRVLSFKTGIISLCKAHLEDKYRYLFKQVASSTGFCDQRRLGLLLH |
| | | DSIQIPRQLGEVASFGGSNIEPSVRSCFQFANNKPEIEAALFLDWMRLEPQ |
| | | SMVWLPVLHRVAAAETAKHQAKCNICKECPIIGFRYRSLKHENYDICQSCF |
| | | FSGRVAKGHKMHYPMVEYCTPTTSGEDVRDFAKVLKNKFRTKRYFAKHPRM |
| | | GYLPVQTVLEGDNMETDTM |
| Dys3978 | 78 | MLWWEEVEDCYEREDVQKKTFTKWVNAQFSKFGKQHIENLFSDLQDGRRLL |
| | | DLLEGLTGQKLPKEKGSTRVHALNNVNKALRVLQNNNVDLVNIGSTDIVDG |
| | | NHKLTLGLIWNIILHWQVKNVMKNIMAGLQQTNSEKILLSWVRQSTRNYPQ |
| | | VNVINFTTSWSDGLALNALIHSHRPDLFDWNSVVCQQSATQRLEHAFNIAR |
| | | YQLGIEKLLDPEDVDTTYPDKKSILMYITSLFQVLPQQVSIEAIQEVEMLP |
| | | RPPKVTKEEHFQLHHQMHYSQQITVSLAQGYERTSSPKPRFKSYAYTQAAY |
| | | VTTSDPTRSPFPSQHLEAPEDKSFGSSLMESEVNLDRYQTALEEVLSWLLS |
| | | AEDTLQAQGEISNDVEVVKDQFHTHEGYMMDLTAHQGRVGNILQLGSKLIG |
| | | TGKLSEDEETEVQEQMNLLNSRWECLRVASMEKQSNLHRVLMDLQNQKLKE |
| | | LNDWLTKTEERTRKMEEEPLGPDLEDLKRQVQQHKVLQEDLEQEQVRVNSL |
| | | THMVVVVDESSGDHATAALEEQLKVLGDRWANICRWTEDRWVLLQDQPDLA |
| | | PGLTTIGASPTQTVTLVTQPVVTKETAISKLEMPSSLMLEVPTHRLLQQFP |
| | | LDLEKFLAWLTEAETTANVLQDATRKERLLEDSKGVKELMKQWQDLQGEIE |
| | | AHTDVYHNLDENSQKILRSLEGSDDAVLLQRRLDNMNFKWSELRKKSLNIR |
| | | SHLEASSDQWKRLHLSLQELLVWLQLKDDELSRQAPIGGDFPAVQKQNDVH |
| | | RAFKRELKTKEPVIMSTLETVRIFLTEQPLEGLEKLYQEPRELPPEERAQN |
| | | VTRLLRKQAEEVNTEWEKLNLHSADWQRKIDETLERLQELQEATDELDLKL |
| | | RQAEVIKGSWQPVGDLLIDSLQDHLEKVKALRGEIAPLKENVSHVNDLARQ |
| | | LTTLGIQLSPYNLSTLEDLNTRWKLLQVAVEDRVRQLHEAHRDFGPASQHE |
| | | LSTSVQGPWERAISPNKVPYYINHETQTTCWDHPKMTELYQSLADLNNVRF |
| | | SAYRTAMKLRRLQKALCLDLLSLSAACDALDQHNLKQNDQPMDILQIINCL |
| | | TTIYDRLEQEHNNLVNVPLCVDMCLNWLLNVYDTGRTGRIRVLSEKTGIIS |
| | | LCKAHLEDKYRYLFKQVASSTGFCDQRRLGLLLHDSIQIPRQLGEVASEGG |
| | | SNIEPSVRSCFQFANNKPEIEAALFLDWMRLEPQSMVWLPVLHRVAAAETA |
| | | KHQAKCNICKECPIIGFRYRSLKHFNYDICQSCFFSGRVAKGHKMHYPMVE |
| | | YCTPTTSGEDVRDFAKVLKNKFRTKRYFAKHPRMGYLPVQTVLEGDNMET |
| Human | 79 | MLWWEEVEDCYEREDVQKKTFTKWVNAQFSKFGKQHIENLFSDLQDGRRLL |
| MD3 | | DLLEGLTGQKLPKEKGSTRVHALNNVNKALRVLQNNNVDLVNIGSTDIVDG |
| | | NHKLTLGLIWNIILHWQVKNVMKNIMAGLQQTNSEKILLSWVRQSTRNYPQ |
| | | VNVINFTTSWSDGLALNALIHSHRPDLFDWNSVVCQQSATQRLEHAFNIAR |
| | | YQLGIEKLLDPEDVDTTYPDKKSILMYITSLFQVLPQQVSIEAIQEVEMLP |
| | | RPPKVTKEEHFQLHHQMHYSQQITVSLAQGYERTSSPKPRFKSYAYTQAAY |
| | | VTTSDPTRSPFPSQHLEAPEDKSFGSSLMESEVNLDRYQTALEEVLSWLLS |
| | | AEDTLQAQGEISNDVEVVKDQFHTHEGYMMDLTAHQGRVGNILQLGSKLIG |
| | | TGKLSEDEETEVQEQMNLLNSRWECLRVASMEKQSNLHRVLMDLQNQKLKE |
| | | LNDWLTKTEERTRKMEEEPLGPDLEDLKRQVQQHKVLQEDLEQEQVRVNSL |
| | | THMVVVVDESSGDHATAALEEQLKVLGDRWANICRWTEDRWVLLQDILLKW |
| | | QRLTEEQCLFSAWLSEKEDAVNKIHTTGFKDQNEMLSSLQKLAVLKADLEK |
| | | KKQSMGKLYSLKQDLLSTLKNKSVTQKTEAWLDNFARCWDNLVQKLEKSTA |
| | | QISQAVTTTQPSLTQTTVMETVTTVTTREQILVKHAQEELPPPPPQKKRQI |
| | | TVDTLERLQELQEATDELDLKLRQAEVIKGSWQPVGDLLIDSLQDHLEKVK |
| | | ALRGEIAPLKENVSHVNDLARQLTTLGIQLSPYNLSTLEDLNTRWKLLQVA |
| | | VEDRVRQLHEAHRDFGPASQHELSTSVQGPWERAISPNKVPYYINHETQTT |
| | | CWDHPKMTELYQSLADLNNVRFSAYRTAMKLRRLQKALCLDLLSLSAACDA |
| | | LDQHNLKQNDQPMDILQIINCLTTIYDRLEQEHNNLVNVPLCVDMCLNWLL |
| | | NVYDTGRTGRIRVLSFKTGIISLCKAHLEDKYRYLFKQVASSTGFCDQRRL |
| | | GLLLHDSIQIPRQLGEVASFGGSNIEPSVRSCFQFANNKPEIEAALFLDWM |
| | | RLEPQSMVWLPVLHRVAAAETAKHQAKCNICKECPIIGFRYRSLKHENYDI |
| | | CQSCFFSGRVAKGHKMHYPMVEYCTPTTSGEDVRDFAKVLKNKFRTKRYFA |
| | | KHPRMGYLPVQTVLEGDNMETPVTLINFWPVDSAPASSPQLSHDDTHSRIE |
| | | HYASRLAEMENSNGSYLNDSISPNESIDDEHLLIQHYCQSLNQDSPLSQPR |
| | | SPAQILISLESEERGELERILADLEEENRNLQAEYDRLKQQHEHKGLSPLP |
| | | SPPEMMPTSPQSPRDAELIAEAKLLRQHKGRLEARMQILEDHNKQLESQLH |
| | | RLRQLLEQPQAEDTM |
| Human | 80 | MLWWEEVEDCYEREDVQKKTFTKWVNAQFSKFGKQHIENLFSDLQDGRRLL |
| MD4 | | DLLEGLTGQKLPKEKGSTRVHALNNVNKALRVLQNNNVDLVNIGSTDIVDG |
| | | NHKLTLGLIWNIILHWQVKNVMKNIMAGLQQTNSEKILLSWVRQSTRNYPQ |
| | | VNVINFTTSWSDGLALNALIHSHRPDLFDWNSVVCQQSATQRLEHAFNIAR |
| | | YQLGIEKLLDPEDVDTTYPDKKSILMYITSLFQVLPQQVSIEAIQEVEMLP |
| | | RPPKVTKEEHFQLHHQMHYSQQITVSLAQGYERTSSPKPRFKSYAYTQAAY |
| | | VTTSDPTRSPFPSQHLEAPEDKSFGSSLMESEVNLDRYQTALEEVLSWLLS |
| | | AEDTLQAQGEISNDVEVVKDQFHTHEGYMMDLTAHQGRVGNILQLGSKLIG |
| | | TGKLSEDEETEVQEQMNLLNSRWECLRVASMEKQSNLHRVLMDLQNQKLKE |
| | | LNDWLTKTEERTRKMEEEPLGPDLEDLKRQVQQHKVLQEDLEQEQVRVNSL |
| | | THMVVVVDESSGDHATAALEEQLKVLGDRWANICRWTEDRWVLLQDILLKW |
| | | QRLTEEQCLFSAWLSEKEDAVNKIHTTGFKDQNEMLSSLQKLAVLKADLEK |
| | | KKQSMGKLYSLKQDLLSTLKNKSVTQKTEAWLDNFARCWDNLVQKLEKSTA |
| | | QISQAVTTTQPSLTQTTVMETVTTVTTREQILVKHAQEELPPPPPQKKRQI |
| | | TVDTLERLQELQEATDELDLKLRQAEVIKGSWQPVGDLLIDSLQDHLEKVK |
| | | ALRGEIAPLKENVSHVNDLARQLTTLGIQLSPYNLSTLEDLNTRWKLLQVA |
| | | VEDRVRQLHEAHRDFGPASQHELSTSVQGPWERAISPNKVPYYINHETQTT |
| | | CWDHPKMTELYQSLADLNNVRFSAYRTAMKLRRLQKALCLDLLSLSAACDA |
| | | LDQHNLKQNDQPMDILQIINCLTTIYDRLEQEHNNLVNVPLCVDMCLNWLL |
| | | NVYDTGRTGRIRVLSFKTGIISLCKAHLEDKYRYLFKQVASSTGFCDQRRL |
| | | GLLLHDSIQIPRQLGEVASFGGSNIEPSVRSCFQFANNKPEIEAALFLDWM |
| | | RLEPQSMVWLPVLHRVAAAETAKHQAKCNICKECPIIGFRYRSLKHENYDI |
| | | CQSCFFSGRVAKGHKMHYPMVEYCTPTTSGEDVRDFAKVLKNKFRTKRYFA |
| | | KHPRMGYLPVQTVLEGDNMETPVTLINFWPVDSAPASSPQLSHDDTHSRIE |
| | | HYASRLAEMENSNGSYLNDSISPNESIDDEHLLIQHYCQSLNQDSPLSQPR |
| | | SPAQILISLESEERGELERILADLEEENRNLQAEYDRLKQQHEHKGLSPLP |
| | | SPPEMMPTSPQSPRDAELIAEAKLLRQHKGRLEARMQILEDHNKQLESQLH |
| | | RLRQLLEQPQAEAKVNGTTVSSPSTSLQRSDSSQPMLLRVVGSQTSDSMGE |
| | | EDLLSPPQDTSTGLEEVMEQLNNSFPSSRGRNTPGKPMREDTM |

In another example, a rAAV comprising a transgene encoding an anti-kallikrein antibody, such as lanadelumab finds use in treating/preventing/managing hereditary angioedema (HAF). In still another example, a rAAV comprising a transgene encoding a lysosomal enzyme finds use in treating/preventing/managing mucopolysaccharidosis. Generally, the rAAV vector is administered systemically, and following transduction, the vector's production of the protein product is enhanced by an expression cassette employing engineered muscle-specific nucleic acid regulatory elements. For example, the rAAV vector may be provided by intravenous, intramuscular, and/or intra-peritoneal administration for secretion of the protein of interest (encoded by the transgene) from muscle as the depot.

With respect to the therapeutic antibodies in Table 4B, the expression cassettes comprising the regulatory sequences operably linked to the transgene encoding the therapeutic antibody may be packaged in an rAAV for delivery that has an AAV8 capsid, an AAV9 capsid or an AAVrh74 capsid for targeting or expression in muscle cells.

In some aspects, the rAAVs of the present invention find use in delivery to target tissues associated with the disorder or disease to be treated/prevented. A disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject the an rAAV where the expression cassette comprises a nucleic acid regulatory element operably linked to a transgene.

Following transduction of target cells, the expression of the protein product is enhanced by employing such liver-specific and muscle-specific expression cassettes. Such enhancement may be measured by the following non-limiting list of determinations such as 1) protein titer by assays known to the skilled person, not limited to sandwich ELISA. Western Blot, histological staining, and liquid chromatography tandem mass spectrometry (LC-MS/MS); 2) protein activity, by assays such as binding assays, functional assays, enzymatic assays and/or substrate detection assays; and/or 3) serum half-life or long-term expression. Enhancement of transgene expression may be determined as efficacious and suitable for human treatment (Hintze, J. P. et al, Biomarker Insights 2011:6 69-78). Assessment of the quantitative and functional properties of a transgene using such in vitro and in vivo cellular, blood and tissue studies have been shown to correlate to the efficacy of certain therapies (Hintze, J. P. et al, 2011, supra), and are utilized to evaluate response to gene therapy treatment of the transgene with the vectors described herein.

rAAV vectors of the invention also can facilitate delivery, in particular, targeted delivery, of transgenes operably linked to the chimeric regulatory sequences described herein, including but not limited to oligonucleotides, drugs, imaging agents, inorganic nanoparticles, liposomes, antibodies to target cells or tissues. The rAAV vectors also can facilitate delivery, in particular, targeted delivery, of non-coding DNA, RNA, or oligonucleotides to target tissues.

The agents may be provided as pharmaceutically acceptable compositions as known in the art and/or as described herein. In some embodiments, the rAAV molecule may be administered alone or in combination with other prophylactic and/or therapeutic agents.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some preferred embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

A rAAV molecule of the invention generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10⁹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the invention can include a single treatment or can include a series of treatments. For example, pharmaceutical compositions comprising an agent of the invention may be administered once a day, twice a day, or three times a day. In some embodiments, the agent may be administered once a day, every other day, once a week, twice a week, once every two weeks, once a month, once every six weeks, once every two months, twice a year, or once per year. It will also be appreciated that the effective dosage of certain agents, e.g., the effective dosage of agents comprising a dual antigen-binding molecule of the invention, may increase or decrease over the course of treatment.

Methods of administering agents of the invention include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In certain embodiments, the transgene is administered intravenously even if intended to be expressed in the CNS, for example, by forming a depot in the liver where the transgene is expressed and secreted into the bloodstream.

In certain embodiments, the agents of the invention are administered intravenously or intramuscularly and may be administered together with other biologically active agents.

In another specific embodiment, agents of the invention may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the invention are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the invention can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Int'l. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref Biomed. Eng., 14:20, 1987; Buchwald et al., Surgery, 88:507, 1980; and Saudek et al., N. Engl. J Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem., 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e.g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In addition, the rAAVs can be used for in vivo delivery of transgenes for scientific studies such as gene knock-down with miRNAs, recombinase delivery for conditional gene deletion, gene editing with CRISPRs, and the like.

### 5.5. Pharmaceutical Compositions and Kits

The invention further provides a pharmaceutical composition comprising a pharmaceutically acceptable carrier and an agent of the invention, said agent comprising a rAAV molecule of the invention comprising a transgene cassette wherein the transgene expression is driven by the chimeric regulatory elements described herein. In preferred embodiments, the pharmaceutical composition comprises rAAV combined with a pharmaceutically acceptable carrier for administration to a subject. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously or intramuscularly. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In preferred embodiments, the agent of the invention is substantially purified (i.e., substantially free from substances that limit its effect or produce undesired side-effects). In a specific embodiment, the host or subject is an animal, preferably a mammal such as non-primate (e.g., cows, pigs, horses, cats, dogs, rats etc.) and a primate (e.g., monkey such as, a cynomolgous monkey and a human). In a preferred embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

### 6.1. Example 1—Cis-Regulatory Element (CRE) Discovery for Muscle-Specific Transgene Expression

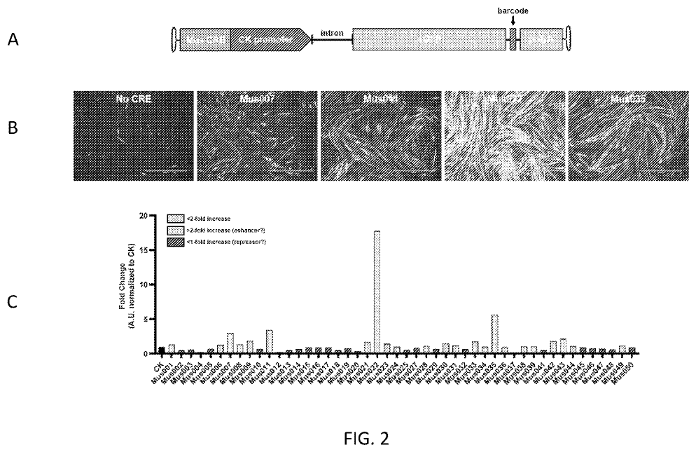

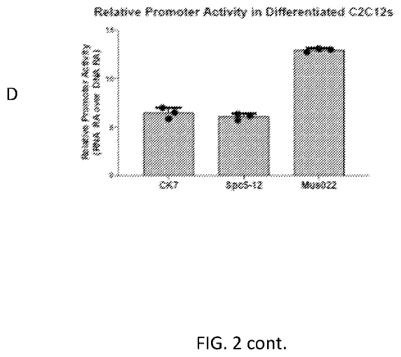

Candidate cis-regulatory element (CRE) sequences derived from regions proximal to genes that are specifically or significantly enriched in skeletal or cardiac muscle were identified in the ENCODE database (Davis, C. et al., 2018 Nucleic Acids Res. The Encyclopedia of DNA elements (ENCODE): data portal update; 46(D1):D794-D801. doi: 10.1093/nar/gkx1081). Compact CRE sequences were cloned upstream of the muscle-specific CK promoter in cis reporter plasmids individually barcoded between the eGFP coding sequence and RBG polyA tail (FIG. 2A). Cis plasmids were mixed to produce one vector prep, or individual preps can be made and then combined.

An AAV “promoter” library was produced and then screened for specificity of the enhancers in vitro, compared to CK promoter alone, by characterizing transgene expression in C2C12 myotubes. Enhancer activity of each CRE was semi-quantitated by calculating the fold-change of transgene expression (eGFP fluorescence) normalized to the control vector (containing CK promoter with no upstream CRE) (FIGS. 2B, 2C). Cis-regulatory modules (CRMs), Mus007, Mus011, Mus022 and Mus035 (also known as composite enhancer/promoters Mus007.CK, Mus011.CK, Mus022.CK and Mus035.CK, respectively) were identified as exhibiting a greater than 2-fold increase normalized to the control plasmid (CK promoter with no upstream CRE), with Mus022 and Mus035 exhibiting significantly increased enhancement of transgene expression and selected for further evaluation. Promoter activity of Mus022 was further evaluated in C2C12 mvoblasts and was represented by the RNA RA divided by the DNA RA for each barcode within each sample (FIG. 2D), which shows that Mus022 exhibits higher relative activity in differentiated C2C12 cells compared to CK7 and Spc5-12 promoter.

### 6.2. Example 2—Evaluation of Muscle-Specific Engineered Promoters in Mice

Selected engineered promoter cassettes containing novel muscle CREs upstream of the CK promoter region were further evaluated in mice. Utilizing the same barcoded AAV libraries (as in Example 1 hereinabove), Cis-regulatory modules (CRMs) that confer unique expression profiles to muscle promoters were evaluated in different tissues following injection of the vector pool in mice. Briefly, AAV vector preparations were generated following a 2L scaled version of suspension-adapted HEK293 cells triple-transfected with helper plasmid, rep/cap plasmid (AAV2/9), and transgene (cis) plasmid. Packaged AAV9 vectors were purified from supernatant and clarified cell lysate using iodixonal gradient ultracentrifugation and formulated. Vector titer was measured using appropriate transgene specific primers via ddPCR.

C57/BL6 mice (n=5) were systemically dosed at 5e13 GC/kg via tail vein injections. Promoter activity (RNA RA divided by DNA RA for each barcode within each sample) was calculated and normalized. Normalized promoter activity is calculated from the normalized RNA RA divided by the normalized DNA RA. Normalized RNA is the RNA RA (out of 1) for each barcode multiplied by the overall RNA copies per TATA box-binding protein (TBP) copies as measured on ddPCR via eGFP primer probes in cDNA. Normalized DNA is the DNA RA (out of 1) for each barcode multiplied by the overall AAV genomes per diploid genome as measured on ddPCR.

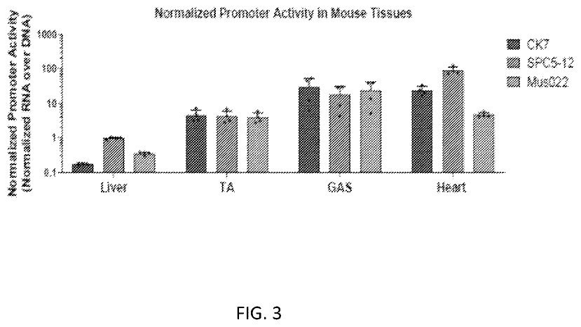

Mus022.CK CRM and Mus035.CK CRM, compared to CK7 and Spc5-12 promoters, displayed similar muscle-specific activity, except surprisingly lower activity in mouse heart tissue, as seen in Table 5 and FIG. 3. Mus022 CRM activity was approximately 5× lower in heart (FIG. 3), indicating that the effect of combining a Mus022 CRE “detargets” CK7 activity promoter in mouse heart.

TABLE 5
Normalized Promoter Activity (Normalized RNA over DNA)

| | Mus035.CK | | | | Mus022.CK | | | | CK7 | |
|---|---|---|---|---|---|---|---|---|---|---|
| Liver | 0.240917 | 0.262396 | 0.209578 | 0.230958 | 0.362915 | 0.372334 | 0.293158 | 0.361685 | 0.178425 | 0.180792 |
| TA | 0.443314 | 0.14861 | 0.113661 | 0.219972 | 5.696699 | 3.98887 | 3.104412 | 2.715334 | 7.106316 | 4.554758 |
| GAS | 1.032821 | 0.138785 | 0.240238 | 1.084653 | 38.46972 | 5.085865 | 13.32478 | 37.56691 | 51.82203 | 6.014851 |
| HEART | 6.254203 | 4.801462 | 4.120469 | 5.038181 | 5.611655 | 4.148835 | 4.09133 | 4.432654 | 33.22559 | 23.54777 |

| | CK7 | | SPC512 | | | |
|---|---|---|---|---|---|---|
| Liver | 0.169106 | 0.149275 | 1.002778 | 1.034325 | 0.878228 | 0.964899 |
| TA | 3.268161 | 3.190378 | 6.694017 | 4.208462 | 2.780011 | 3.13767 |
| GAS | 12.27395 | 44.25576 | 29.47996 | 4.344695 | 8.775262 | 28.24647 |
| HEART | 16.28074 | 20.49869 | 120.8451 | 84.80044 | 73.58515 | 72.34128 |

### 6.3. Example 3: Single Vector Evaluation of CK7 Versus Heart “Detargeted” Mus022 Promoters in Mdx Mice

Individual vector preps of AAV9 packaging CK7.μDys and Mus022.Dys were produced similarly to the method mentioned above. The cis plasmid encoded for a microdystrophin (μDys) protein. C57BL/10ScSn-Dmdmdx/J (mdx) mice (n=5) were systemically dosed at 5e13 GC/kg for each vector. Tissues were harvested 5 weeks post dosing. Briefly, 10 mdx mice, 5 in each group as follows: Group 1=AAV9.CK7.μDys and Group 2=AAV9.Mus022.CK.μDys. Mice were systemically dosed at 5e13GC/kg via tail vein injections for each group and necropsied 5 weeks post injection. Tissues were harvested and snap frozen in isopentane/LN2 double bath, then RNA extracted via KingFisher Apex, and DNA extracted via Qiagen DNEASY kit. cDNA was subsequently synthesized using MAXIMA RT kit with dsDNASEI and ddPCR duplex assay was utilized to assess normalized RNA and DNA levels. Heart was sectioned and stained with DysB and laminin.

### A. Quantification of μDys Protein

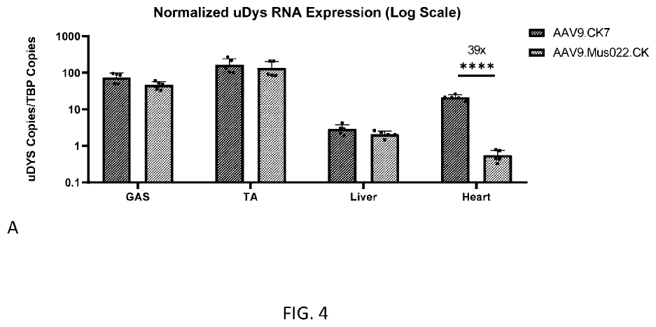

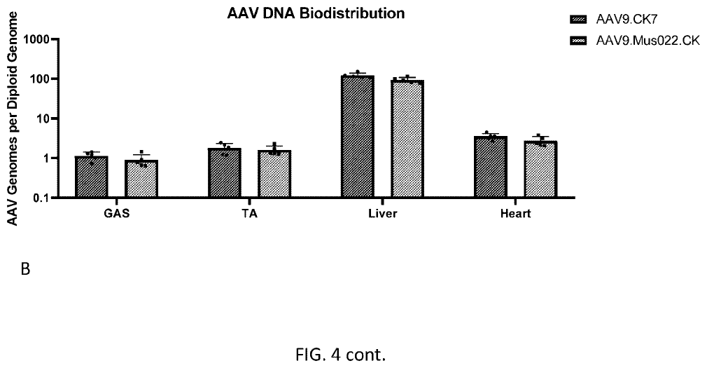

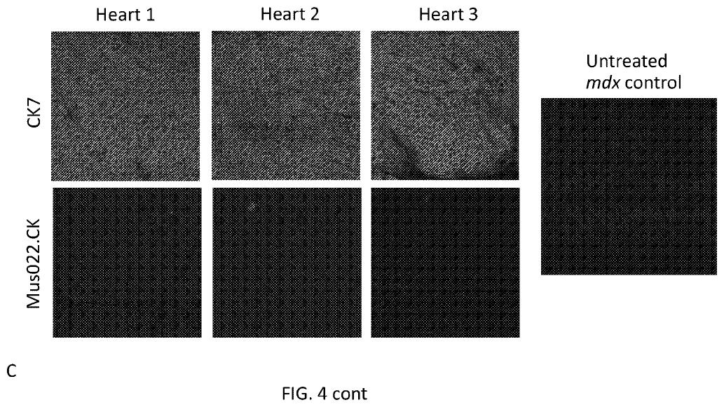

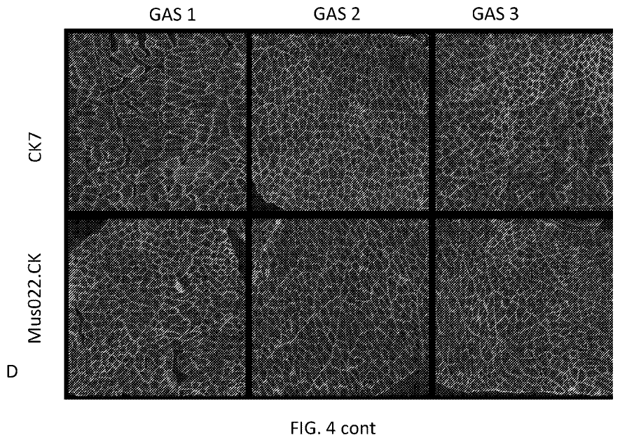

cDNA copies per TBP copies were analyzed in gastrocnemius (GAS), tibialis anterior (TA), quadricep (Quad), Liver, and Heart via ddPCR as represented in FIG. 4A.

### B. Quantification of AAV Genomes Per Diploid Genome in Tissues

DNA biodistribution for GAS, TA, Quad, Liver, and Heart as represented in FIG. 4B.

### C. Immunofluorescent (IF) Staining for Human Dystrophin in Heart

IF staining for human dystrophin reveals similar Mus022 detargeting in mouse heart on protein level in heart tissue sections comparing CK7.pDvs and Mus022.μDys-treated mice, as represented in FIG. 4C.

### D. IF Staining for Human Dystrophin in Gastrocnemius (GAS)

IF staining for human dystrophin in GAS sections from CK7.μDys and Mus022.μDys-treated mice illustrated similar protein levels, as represented in FIG. 4D.

Overall Mus022.CK is a ˜800 bp muscle-specific promoter consisting of a 347 bp CRE (SEQ ID NO: 10) and the mouse creatine kinase core promoter. The upstream Mus022 CRE was isolated from the most recent assembly (GRCh38) of the human genome and is derived from the MYLPF gene (also known as MYL11, MLC2B) which encodes the myosin light chain, phosphorylatable, fast skeletal muscle protein. Although Mus022.CK exhibited slightly weaker RNA expression in mdx skeletal muscle versus CK7, it exhibited significantly less RNA expression in the mdx heart providing a gene expression profile that is advantageous for linking operably the Mus022.CK promoter to therapeutic genes delivered preferentially to skeletal muscle. IF staining confirmed the large difference between the Mus022.CK and CK7 promoters in microdystrophin expression in mdx heart.

### 6.4. Example 4: Small Muscle-Specific Promoters

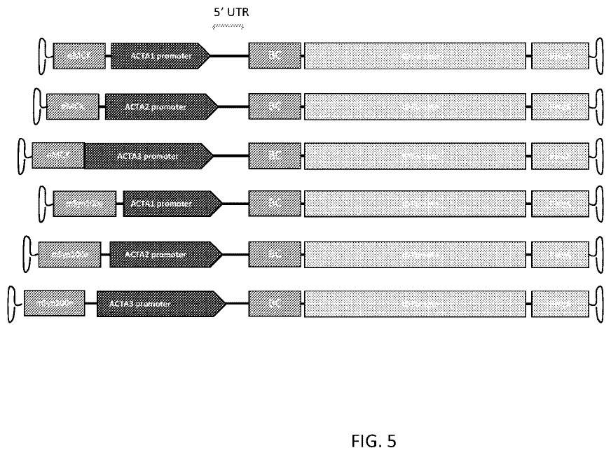

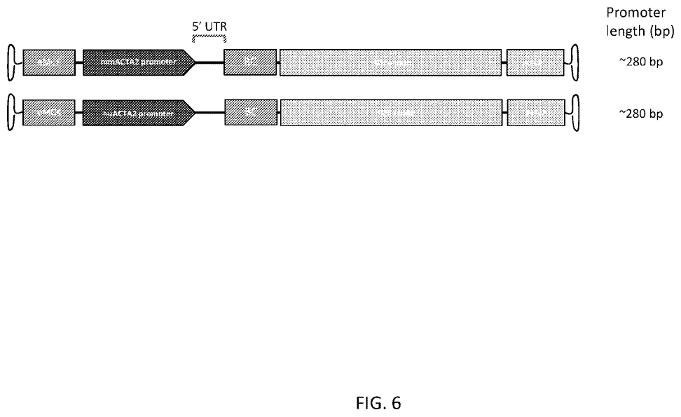

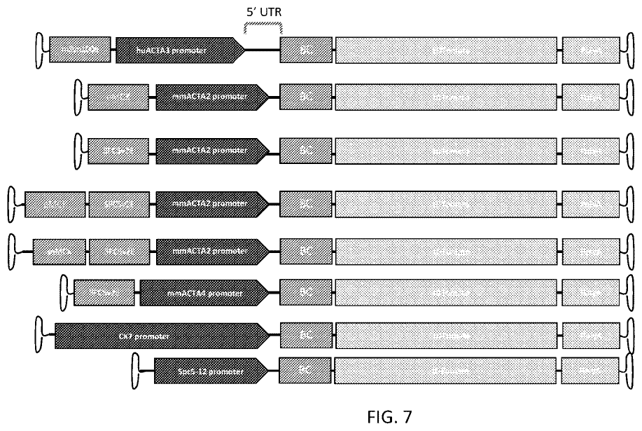

An evaluation of another AAV library, constructed analogously as above to make a vector pool, was utilized to evaluate various hybrid promoters driving a fluorescent marker transgene (FIG. 5). Muscle promoter pools were evaluated in mdx mice (n=5) at a systemic dose of 8e13GC/kg. The Engineered Muscle Promoters (EMPs) are novel muscle promoters designed using bioinformatics and rational engineering. Briefly, a library of Cis-Regulatory Element (CRE) sequences were derived from regions proximal to genes that are specifically or significantly enriched in skeletal or cardiac muscle, auch as alpha (α)-actin (ACTA) genes. Sequence databases such as ENCODE and TRANSFAC were utilized to assist in identification of important genetic features for muscle-specific transcriptional activity, such as positive and negative regulators of transcription, transcription factor binding sites, and the like. Novel promoters were created by engineering each CRE in a composite sequence including upstream enhancers, such as eMCK, with or without untranslated regions (UTRs), and cloned into an AAV vector transgene cassette with a unique barcode to allow for NGS-based evaluation of transgene expression (FIGS. 5 through 7).

Several iterations of EMP with novel enhancer regions were designed to further improve transcriptional activity in muscle and include tandem promoters, such as SPC5v2 upstream of the ACTA2 CRE, and include an untranslated region identified between the TSS and the start codon of the ACTA2 gene (the UTR within the CRE does not contain any ATGs that could aberrantly initiate early transcription of the transgene). In some EMPs, muscle expression is improved while either increasing or decreasing transcriptional activity in heart. The EMPs are also compact in size, e.g. less than −600 nucleotide bases, compared to a CAG promoter which is over 1600 bases in length. Small promoters are important in gene therapy so that the gene cassette size (limited to ˜4.7 kb for AAV) can be maximized with other regulatory elements, RNAi comopents, RNA stabilizing elements, or large transgenes.

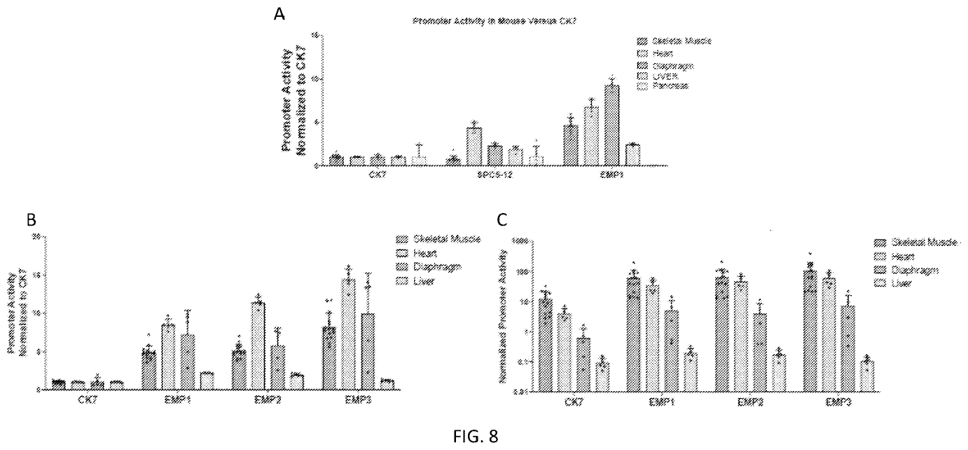

A comparison of CK7, SPC5-12, and eMCK.mmACTA2shortUTR muscle promoters using NGS in skeletal muscles (GAS, quad, TA), heart, diaphragm, liver, and pancreas was performed and activity in each tissue for SPC5-12 and eMCK.mmACTA2shortUTR is normalized to CK7 (FIG. 8A).

A comparison of second generation of engineered muscle promoters, SPC5v2.mmACTA2shortUTR and eMCK.SPC5v2.mmACTA2shortUTR with eMCK.mmACTA2shortUTR and CK7 was also performed. The activity is normalized to CK7 using NGS (FIG. 8B). Normalized promoter activity was calculated using RNA copies/TBP copy and AAV genomes per diploid genome (FIG. 8C). These Engineered Muscle Promoters (EMPs) exhibited enhanced activity in mouse muscle when compared to CK7. The small size of these promoters, particularly eMCK.mmACTA2shortUTR (˜280 bp), allows for the expression of large transgenes via rAAV vectors, which is highly useful due to constraint of the limited genome size that can be packaged by AAV. These new promoters have the potential for potent transcriptional activity in human muscle, which could ultimately decrease effective dose levels for muscle gene therapies.

### 6.5. Example 5: In Vivo Studies Measuring Promoter Activity of Small Muscle-Specific Promoters Driving Expression of a Muscle Protein

Eleven muscle specific promoters (Table) selected from previous studies were cloned into barcoded microdystrophin AAV transgene plasmids, similarly to FIGS. 5-7 except that the transgene downstream of the promoter encodes for a microdystrophin protein, and each microdystrophin plasmid contained a 3′ untranslated region (UTR) and barcode between the transgene and polvA sequences. Microdystrophin is an engineered protein meant to mimic the function of dystrophin, an important structural muscle protein with important functions including maintaining muscle membrane integrity. Individual AAV vector preps (packaged into a AAVhu32 capsid) for each barcoded muscle promoter transgene plasmid were produced by triple transfection and purified by iodixanol gradient. AAV preps were pooled at equal contributions to produce a single AAV muscle promoter pool comprised of these eleven unique muscle promoters driving expression of barcoded microdystrophin mRNAs.

TABLE 6
Promoters selected for in vivo studies

| Promoter name | SEQ ID NO: |
|---|---|
| CK7 | 1 |
| SPC5v2 | 7 |
| eMCK.HUACTA3 | 32 |
| mSYN100E.huACTA3 | 38 |
| eMCK.MMACTA2shortutr | 39 |
| spc5v2.MMACTA2shortUTR | 41 |
| eMCK.SPC5v2.MMACTA2shortUTR | 42 |
| seMCK.SPC5v2.MMACTA2shortUTR | 68 |
| eMCK.fSPC5v2.MMACTA2shortUTR | 69 |
| SPC5v2.MMACTA4 | 45 |
| eMCK.SPC5v2.HuACTA2midUTR | 51 |

Five 6-week old mice were dosed with the AAV muscle promoter pool at 1×10¹⁴ gc/kg per mouse. Tissues were harvested 4 weeks post dosing and DNA/RNA were extracted from tissues taken. Relative abundance (RA) of barcodes in each sample (both RNA and DNA) was determined by next generation sequencing (NGS) of barcode amplicons using the Illumina MiSeq platform. Relative promoter activity for each muscle promoter in each tissue was determined by dividing RNA relative abundance (RA) by DNA RA for each promoter. Normalized promoter activity was calculated by dividing normalized RNA by normalized DNA. Normalized RNA is the RNA RA (out of 1) for each barcode multiplied by the overall RNA copies per TATA box-binding protein (TBP) copies as measured on ddPCR via eGFP primer probes in cDNA. Normalized DNA was calculated similarly, however with total AAV genomes per diploid genome as measured on ddPCR.

Two NHPs were also dosed at 1×10¹⁴ gc/kg with the AAV muscle promoter pool and tissues were collected three months post dosing. Analysis of promoter activity was performed similarly to the mouse study.

### Results

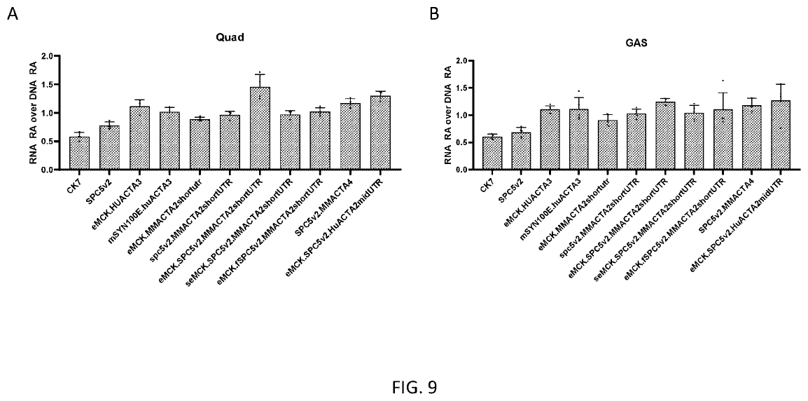

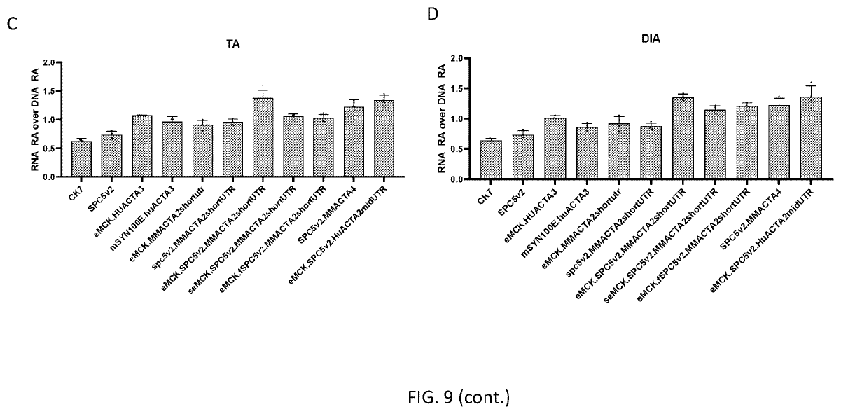

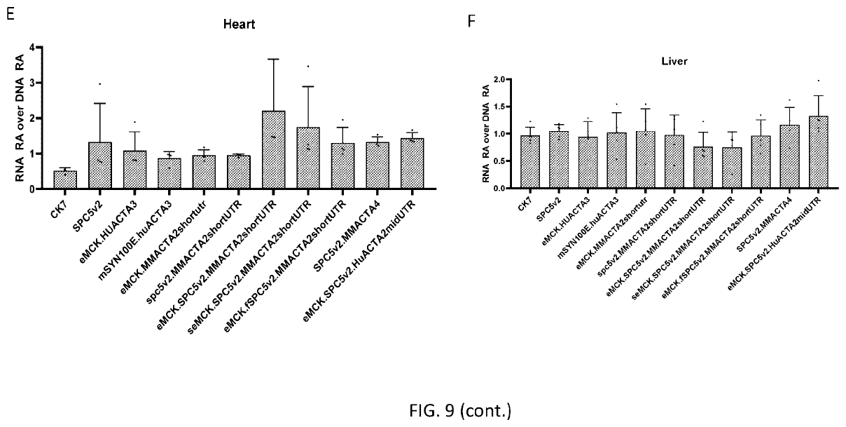

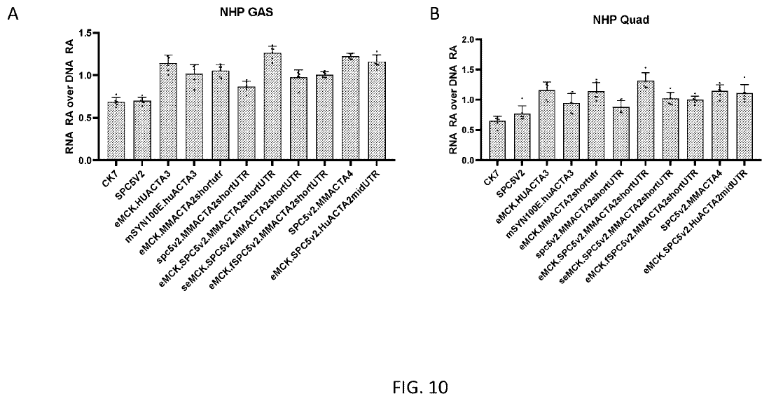

Muscle promoters were assessed for activity in different tissues against two controls, CK7 and SPC5v2. In this study, novel hybrid promoters were determined to have increased transgene expression in skeletal muscle and diaphragm, while having low expression in cardiac tissue, or high expression in cardiac tissue. Depending on the transgene to be delivered via gene therapy, there will be therapeutic utility for low or high expression promoters in heart, especially relatively small (less than ˜1000 bp) promoters. Results from NGS analysis of different mouse tissues revealed that the eMCK.SPC5v2.MMACTA2shortUTR, SPC5v2.MMACTA4, and eMCK.SPC5v2.HuACTA2midUTR promoters drove the strongest expression in skeletal muscle and diaphragm (FIGS. 9A-D), ranging from 2-3 fold better expression compared to the control muscle promoter, CK7. In heart, the eMCK.SPC5v2.MMACTA2shortUTR promoter drove the most expression by up to 4 fold more than CK7 (FIG. 9E). Other promoters, such as SPC5v2.MMACTA4 and eMCK.SPC5v2.HuACTA2midUTR, were still more potent than CK7, with nearly 2-fold more activity. NGS results from liver samples revealed that all the promoters in the pool had similar activity versus CK7 and SPC5v2 (FIG. 9F), which are well known to be muscle specific and liver detargeted promoters.

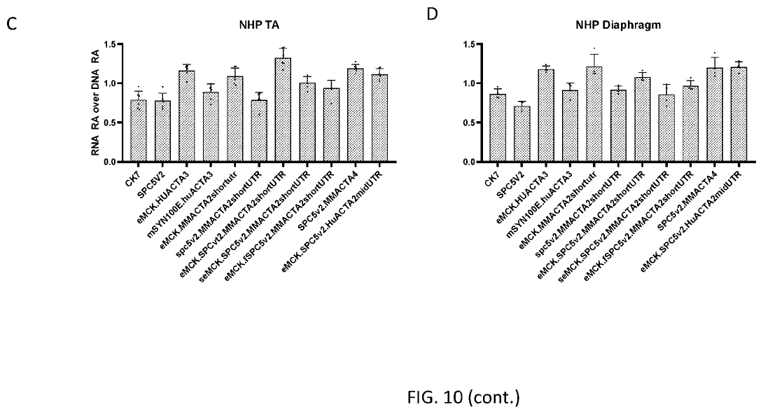

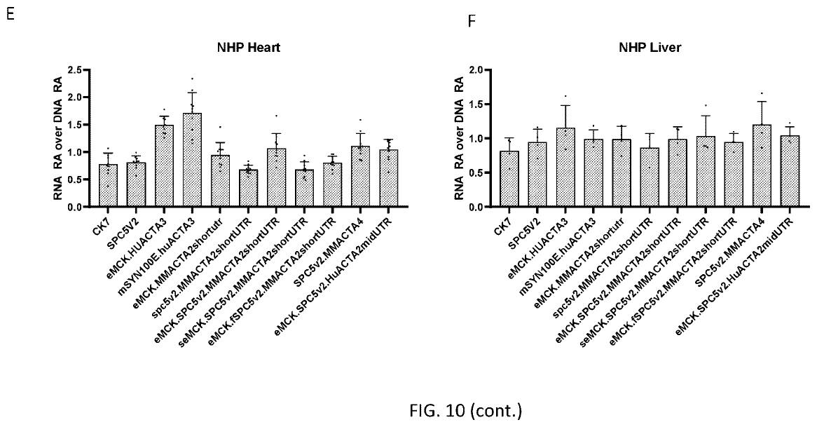

Results from the NHP study were similar, indicating that the same three promoters, eMCK.SPC5v2.MMACTA2shortUTR, SPC5v2.MMACTA4, and eMCK.SPC5v2.HuACTA2midUTR, were still the top performers in skeletal muscle in terms of promoter activity. These promoters were nearly two-fold better than CK7 in gastrocnemius (GAS), quadriceps (Quad), and tibalis anterior (TA) (FIGS. 10A-C). Interestingly, the most active promoters in NHP heart were different from the top performers in mouse heart, with eMCK.HUACTA3 and mSYN100E.HuACTA3 being the most potent heart promoters in this study (FIG. 10E). In diaphragm, the best promoter was eMCK.MMACTA2shortutr, outperforming CK7 by a slight margin (FIG. 10D). Finally, all the promoters in this study were similar in liver activity compared to CK7, indicating low promoter activity in NHP liver for all promoters tested (FIG. 10F).

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We claim:

1. A recombinant expression cassette comprising a composite nucleic acid regulatory element comprising a) Muscle cis regulatory element (CRE), and b) a CK promoter, a Spc5-12 promoter, or variant thereof, or an ACTA core promoter, operably linked to a transgene.

2. The recombinant expression cassette of claim 1, wherein the muscle CRE is Mus022 (SEQ ID NO: 10), Mus077 (SEQ ID NO: 11), Mus011 (SEQ ID NO: 12) or Mus035 (SEQ ID NO: 13).

3. The recombinant expression cassette of claim 2, wherein the muscle CRE is Mus022 (SEQ ID NO: 10).

4. The recombinant expression cassette of any one of claims 1 to 3, wherein the composite nucleic acid regulatory element is Mus022.CK (SEQ ID NO: 9).

5. The recombinant expression cassette of claim 1, wherein the muscle CRE is eMCK (SEQ ID NO: 4), seMCK (SEQ ID NO: 5), mSYNl00E (SEQ ID NO: 6) or SPc5v2 (SEQ ID NO: 7).

6. The recombinant expression cassette of any one of claims 1 to 3 or 5, wherein the ACTA promoter is mmACTA1 (SEQ ID NO: 14), mmACTA2 (SEQ ID NO: 15), mmACTA3 (SEQ ID NO: 16), mmACTA4 (SEQ ID NO: 17), huACTA1 (SEQ ID NO: 18), huACTA2 (SEQ ID NO: 19), huACTA3 (SEQ ID NO: 20), mmACTA2-shortUTR (SEQ ID NO: 21), mmACTA2-midUTR (SEQ ID NO: 22), huACTA2-shortUTR (SEQ ID NO: 23), huACTA2-midUTR (SEQ ID NO: 24), mmACTA3-shortUTR (SEQ ID NO: 25) or huACTA3-midUTR (SEQ ID NO: 26).

7. A recombinant expression cassette comprising a composite nucleic acid regulatory element comprising or consisting of a nucleic acid sequence having a) 99%, 95%, 90%, 85% or 80% sequence identity with any one of SEQ ID NOs: 9, 27-54, 68 and 69 and/or having b) 1 to 10 nucleotide substitutions compared to any one of SEQ ID NOs: 9, 27-54, 68 and 69, wherein the composite nucleic acid regulatory element retains biological activity of any one of SEQ ID NOs: 9, 27-54, 68 and 69.

8. The recombinant expression cassette of claim 7, wherein the composite nucleic acid regulatory element comprises any one of the nucleotide sequences of SEQ ID NO: 9, 27-54, 68, or 69.

9. The recombinant expression cassette of claim 8, wherein the composite nucleic acid regulatory element consists of any one of the nucleotide sequences of SEQ ID NO: 9, 27-54, 68, or 69

10. The recombinant expression cassette of any one of claims 1 to 9, further comprising an intron sequence between the composite nucleic acid regulatory element and the transgene.

11. The recombinant expression cassette of any one of claims 1 to 10, wherein the transgene is a gene or nucleic acid encoding a therapeutic listed in Tables 4A-4C.

12. The recombinant expression cassette of claim 11, wherein the transgene is a muscle-derived protein.

13. The recombinant expression cassette of claim 11, wherein the transgene is a minidystrophin gene or a microdystrophin gene.

14. The recombinant expression cassette of claim 13, wherein microdystrophin is Dys1 (SEQ ID NO: 73), Dys3 (SEQ ID NO: 74), Dys5 (SEQ ID NO: 75), human MD1 (R4-R23/ACT) (SEQ ID NO: 76), Human microdystrophin (SEQ ID NO: 77), Dys3978 (SEQ ID NO: 78), Human MD3 (SEQ ID NO: 79) or Human MD4 (SEQ ID NO: 80).

15. The recombinant expression cassette of any one of claims 1 to 10, wherein the transgene is a gene or nucleic acid encoding a therapeutic antibody listed in Table 4B, or antigen binding fragment thereof.

16. A vector comprising the recombinant expression cassette of any one of claims 1 to 15.

17. The vector of claim 16, further comprising AAV ITRs flanking the expression cassette.

18. The vector of claim 16 or claim 17 wherein the recombinant expression cassette is suitable for packaging in an ssAAV or scAAV vector.

19. An rAAV particle comprising the vector of any one of claims 16 to 18, and a capsid protein from an AAV capsid serotype selected from AAV1, AAV1, AAV2, AAV3, AAV4, AAV5, AAV6, AAV7, AAV8, AAV9, AAV10, AAV-11, AAV-12, AAV-13, AAV-14, AAV-15, AAV-16, AAV.rh8, AAV.rh10, AAV.rh20, AAV.rh39, AAV.Rh74, AAV.RHM4-1, AAV.hu37, AAV.hu32, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

20. A method for enhancing expression of a transgene in a subject, said method comprising delivering a viral vector comprising a recombinant expression cassette comprising a composite nucleic acid regulatory element comprising in a 5′ to 3′ arrangement a) a Mus022 sequence; b) at least one muscle-specific promoter; c) a transgene; and d) a polyadenylation signal sequence.

21. The method of claim 20 wherein the muscle specific promoter is CK promoter (SEQ ID NO: 8).

22. The method of claim 20 or claim 21, wherein the viral vector is administered intravenously or intramuscularly.

23. The method of any one of claims 20 to 22, wherein transgene expression is enhanced in circulation or systemically.

24. The method of any one of claims 20 to 23, wherein transgene expression is enhanced in liver or skeletal muscle.

25. A method of treatment comprising delivering a rAAV comprising the recombinant expression cassette of any one of claims 1-15, or the vector of any one of claims 16 to 18, or delivering the rAAV of claim 19.


SEQUENCE LISTING (80 sequences)

SEQ ID NO: 1 - DNA, 587 bp, synthetic construct
  source 1..587: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcggtacccgggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 2 - DNA, 400 bp, synthetic construct
  source 1..400: mol_type = other DNA
cggccgtccgccctcggcaccattcctcacgacacccaaatatggcgacgggtgaggaatggtggggagttatttttagagcggtgaggaatggtgggcaggcagcaggtgttgggggagttatttttagagcggggagttatttttagagcggtgaggaatggtggacacccaaatatggcgacgggtgaggaatggtgccgtcgccatatttgggtgtcccgtccgccctcggccggggccgcattcctgggggccgggcggtgctcccgcccgcctcgataaaaggctccggggccggcggcggcccacgagctacccggaggagcgggaggcgtctctgccagcggtccgacgcgcagtcagcaccaggtaggtgggcaccgcgccgtgccgtgcc

SEQ ID NO: 3 - DNA, 328 bp, synthetic construct
  source 1..328: mol_type = other DNA
ggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagc

SEQ ID NO: 4 - DNA, 144 bp, synthetic construct
  source 1..144: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 5 - DNA, 89 bp, synthetic construct
  source 1..89: mol_type = other DNA
tgcctggttataattaacccagacatgtggctgccccccccccccccaacacctgctgcctctaaaaataaccctgtccctggtggatc

SEQ ID NO: 6 - DNA, 233 bp, synthetic construct
  source 1..233: mol_type = other DNA
cggccgtccgccctcggcaccattcctcacgacacccaaatatggcgacgggtgaggaatggtggggagttatttttagagcggtgaggaatggtgggcaggcagcaggtgttgggggagttatttttagagcggggagttatttttagagcggtgaggaatggtggacacccaaatatggcgacgggtgaggaatggtgccgtcgccatatttgggtgtcccgtccgccctc

SEQ ID NO: 7 - DNA, 217 bp, synthetic construct
  source 1..217: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccct

SEQ ID NO: 8 - DNA, 433 bp, synthetic construct
  source 1..433: mol_type = other DNA
ccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 9 - DNA, 795 bp, synthetic construct
  source 1..795: mol_type = other DNA
ctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagagggagcggagaggggtacccgggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 10 - DNA, 347 bp, synthetic construct
  source 1..347: mol_type = other DNA
ctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagagggagcgg

SEQ ID NO: 11 - DNA, 351 bp, synthetic construct
  source 1..351: mol_type = other DNA
aaccattgtgggcagccagcctggcgggagaggctagggcacgaagcgccgcccagttaccatggtaacactccctcgtcccaaccccgtcccagtttactggcccaaggtaaggccgggagccgtactccagggcgactgggaggatccactccctgccgcggctctccctccagtggactgccttcccgcctgcctggcggtgggagggcgcggcagagggacgtcacatccgggcgggttggtgagttccggtatttcagggcgtagcaggcggaagtaagggtgagaggaggctgcaacgccgagcggaggaggcaggaaccggagcgcgagcagtagctgggtggg

SEQ ID NO: 12 - DNA, 346 bp, synthetic construct
  source 1..346: mol_type = other DNA
ctcccctcttcctcttcttttaaaacatccatagagctgcccagaatgagtggaaagtttcagggtcagggagaacctcttccctcccccactttccattgtctaaaatcagcctcagctgttgctagatattttttttgctggtgatgtcagttcatcaaagcgtctcaccctgtgttcccccaggagaacaatgatggggtggagggagctggaaagaccatattaggaacaaaaggtggagctatgagcacagataaagactcaagtctggggacctcctggtcactcaggcagcagccccttctttcttgccccagtctccagttctccagtgttcacaggt

SEQ ID NO: 13 - DNA, 330 bp, synthetic construct
  source 1..330: mol_type = other DNA
ggcactggggtgtgtctccttaggacacccctgcccattactctctccttctccaaatggggagcatggctggggctccctaacctcctgcttgcgaggcctctctctggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagcccctgtggctccagtcccctgggatctgttggcttctggctggagatgaagattagggcagaggaga

SEQ ID NO: 14 - DNA, 188 bp, synthetic construct
  source 1..188: mol_type = other DNA
gaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 15 - DNA, 215 bp, synthetic construct
  source 1..215: mol_type = other DNA
agtgcccgacacccaaatatggcttgggaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 16 - DNA, 367 bp, synthetic construct
  source 1..367: mol_type = other DNA
agttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 17 - DNA, 181 bp, synthetic construct
  source 1..181: mol_type = other DNA
agttgggaggggcagacagctggggatactctccatatacggcctggtccggtccagtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 18 - DNA, 170 bp, synthetic construct
  source 1..170: mol_type = other DNA
gaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 19 - DNA, 196 bp, synthetic construct
  source 1..196: mol_type = other DNA
gtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 20 - DNA, 338 bp, synthetic construct
  source 1..338: mol_type = other DNA
aggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 21 - DNA, 126 bp, synthetic construct
  source 1..126: mol_type = other DNA
agtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 22 - DNA, 165 bp, synthetic construct
  source 1..165: mol_type = other DNA
agtgcccgacacccaaatatggcttgggaagggcagcaacattccttcggggcggtgtggggagagctcccgggactatataaaaacctgtgcaaggggacaggcggtcacacggacgtgaagcctcacttcctaccctcggcacccagggccagagtcagagca

SEQ ID NO: 23 - DNA, 122 bp, synthetic construct
  source 1..122: mol_type = other DNA
gtgcccaacacccaaatatggctcgagaagggcagcgacattcctgcggggtggcgcggagggaatgcccgcgggctatataaaacctgagcagagggacaagcggccaccgcagcggacag

SEQ ID NO: 24 - DNA, 159 bp, synthetic construct
  source 1..159: mol_type = other DNA
gtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgacca

SEQ ID NO: 25 - DNA, 278 bp, synthetic construct
  source 1..278: mol_type = other DNA
agttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 26 - DNA, 301 bp, synthetic construct
  source 1..301: mol_type = other DNA
aggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgacca

SEQ ID NO: 27 - DNA, 342 bp, synthetic construct
  source 1..342: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcggtacccggggaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 28 - DNA, 369 bp, synthetic construct
  source 1..369: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcggtacccgggagtgcccgacacccaaatatggcttgggaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 29 - DNA, 521 bp, synthetic construct
  source 1..521: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcggtacccgggagttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 30 - DNA, 324 bp, synthetic construct
  source 1..324: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcggtacccggggaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 31 - DNA, 350 bp, synthetic construct
  source 1..350: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcggtacccggggtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 32 - DNA, 492 bp, synthetic construct
  source 1..492: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcggtacccgggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 33 - DNA, 431 bp, synthetic construct
  source 1..431: mol_type = other DNA
cggccgtccgccctcggcaccattcctcacgacacccaaatatggcgacgggtgaggaatggtggggagttatttttagagcggtgaggaatggtgggcaggcagcaggtgttgggggagttatttttagagcggggagttatttttagagcggtgaggaatggtggacacccaaatatggcgacgggtgaggaatggtgccgtcgccatatttgggtgtcccgtccgccctcggtacccggggaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 34 - DNA, 458 bp, synthetic construct
  source 1..458: mol_type = other DNA
cggccgtccgccctcggcaccattcctcacgacacccaaatatggcgacgggtgaggaatggtggggagttatttttagagcggtgaggaatggtgggcaggcagcaggtgttgggggagttatttttagagcggggagttatttttagagcggtgaggaatggtggacacccaaatatggcgacgggtgaggaatggtgccgtcgccatatttgggtgtcccgtccgccctcggtacccgggagtgcccgacacccaaatatggcttgggaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 35 - DNA, 610 bp, synthetic construct
  source 1..610: mol_type = other DNA
cggccgtccgccctcggcaccattcctcacgacacccaaatatggcgacgggtgaggaatggtggggagttatttttagagcggtgaggaatggtgggcaggcagcaggtgttgggggagttatttttagagcggggagttatttttagagcggtgaggaatggtggacacccaaatatggcgacgggtgaggaatggtgccgtcgccatatttgggtgtcccgtccgccctcggtacccgggagttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 36 - DNA, 413 bp, synthetic construct
  source 1..413: mol_type = other DNA
cggccgtccgccctcggcaccattcctcacgacacccaaatatggcgacgggtgaggaatggtggggagttatttttagagcggtgaggaatggtgggcaggcagcaggtgttgggggagttatttttagagcggggagttatttttagagcggtgaggaatggtggacacccaaatatggcgacgggtgaggaatggtgccgtcgccatatttgggtgtcccgtccgccctcggtacccggggaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 37 - DNA, 439 bp, synthetic construct
  source 1..439: mol_type = other DNA
cggccgtccgccctcggcaccattcctcacgacacccaaatatggcgacgggtgaggaatggtggggagttatttttagagcggtgaggaatggtgggcaggcagcaggtgttgggggagttatttttagagcggggagttatttttagagcggtgaggaatggtggacacccaaatatggcgacgggtgaggaatggtgccgtcgccatatttgggtgtcccgtccgccctcggtacccggggtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 38 - DNA, 581 bp, synthetic construct
  source 1..581: mol_type = other DNA
cggccgtccgccctcggcaccattcctcacgacacccaaatatggcgacgggtgaggaatggtggggagttatttttagagcggtgaggaatggtgggcaggcagcaggtgttgggggagttatttttagagcggggagttatttttagagcggtgaggaatggtggacacccaaatatggcgacgggtgaggaatggtgccgtcgccatatttgggtgtcccgtccgccctcggtacccgggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 39 - DNA, 280 bp, synthetic construct
  source 1..280: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcggtacccgggagtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 40 - DNA, 276 bp, synthetic construct
  source 1..276: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcggtacccggggtgcccaacacccaaatatggctcgagaagggcagcgacattcctgcggggtggcgcggagggaatgcccgcgggctatataaaacctgagcagagggacaagcggccaccgcagcggacag

SEQ ID NO: 41 - DNA, 353 bp, synthetic construct
  source 1..353: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggagtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 42 - DNA, 500 bp, synthetic construct
  source 1..500: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcgtcggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggagtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 43 - DNA, 539 bp, synthetic construct
  source 1..539: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcgtcggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggagtgcccgacacccaaatatggcttgggaagggcagcaacattccttcggggcggtgtggggagagctcccgggactatataaaaacctgtgcaaggggacaggcggtcacacggacgtgaagcctcacttcctaccctcggcacccagggccagagtcagagca

SEQ ID NO: 44 - DNA, 505 bp, synthetic construct
  source 1..505: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggagttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 45 - DNA, 408 bp, synthetic construct
  source 1..408: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggagttgggaggggcagacagctggggatactctccatatacggcctggtccggtccagtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 46 - DNA, 408 bp, synthetic construct; identical to SEQ ID NO: 45
  source 1..408: mol_type = other DNA

SEQ ID NO: 47 - DNA, 503 bp, synthetic construct
  source 1..503: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaag

SEQ ID NO: 48 - DNA, 528 bp, synthetic construct
  source 1..528: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgacca

SEQ ID NO: 49 - DNA, 386 bp, synthetic construct
  source 1..386: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccggggtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgacca

SEQ ID NO: 50 - DNA, 675 bp, synthetic construct
  source 1..675: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcgtcggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgacca

SEQ ID NO: 51 - DNA, 533 bp, synthetic construct
  source 1..533: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcgtcggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccggggtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgacca

SEQ ID NO: 52 - DNA, 441 bp, synthetic construct
  source 1..441: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggaggggcaaacccgctagggagacactccatatacggcccggcccgcgttacctgggtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgacca

SEQ ID NO: 53 - DNA, 565 bp, synthetic construct
  source 1..565: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcc

SEQ ID NO: 54 - DNA, 594 bp, synthetic construct
  source 1..594: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggagttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtcacacggacgtgaagcctcacttcctaccctcggcacccagggccagagtcagagcagcacttagggtggaggtggggagggtgacctcgagacccagcaaagaaag

SEQ ID NO: 55 - DNA, 100 bp, synthetic construct
  source 1..100: mol_type = other DNA
aggttaatttttaaaaagcagtcaaaagtccaagtggcccttggcagcatttactctctctgtttgctctggttaataatctcaggagcacaaacattcc

SEQ ID NO: 56 - DNA, 200 bp, synthetic construct
  source 1..200: mol_type = other DNA
aggttaatttttaaaaagcagtcaaaagtccaagtggcccttggcagcatttactctctctgtttgctctggttaataatctcaggagcacaaacattccaggttaatttttaaaaagcagtcaaaagtccaagtggcccttggcagcatttactctctctgtttgctctggttaataatctcaggagcacaaacattcc

SEQ ID NO: 57 - DNA, 288 bp, synthetic construct
  source 1..288: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 58 - DNA, 432 bp, synthetic construct
  source 1..432: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 59 - DNA, 199 bp, synthetic construct
  source 1..199: mol_type = other DNA
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggc

SEQ ID NO: 60 - DNA, 433 bp, synthetic construct; identical to SEQ ID NO: 8
  source 1..433: mol_type = other DNA

SEQ ID NO: 61 - DNA, 174 bp, synthetic construct
  source 1..174: mol_type = other DNA
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaag

SEQ ID NO: 62 - DNA, 1676 bp, synthetic construct
  source 1..1676: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 63 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 64 - DNA, 82 bp, synthetic construct
  source 1..82: mol_type = other DNA
gtgagtatctcagggatccagacatggggatatgggaggtgcctctgatcccagggctcactgtgggtctctctgttcacag

SEQ ID NO: 65 - DNA, 97 bp, synthetic construct
  source 1..97: mol_type = other DNA
gtaagtttagtctttttgtcttttatttcaggtcccggatccggtggtggtgcaaatcaaagaactgctcctcagtggatgttgcctttacttctag

SEQ ID NO: 66 - DNA, 55 bp, synthetic construct
  source 1..55: mol_type = other DNA
ataaaggaaatttattttcattgcaatagtgtgttggaattttttgtgtctctca

SEQ ID NO: 67 - DNA, 127 bp, synthetic construct
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 68 - DNA, 445 bp, synthetic construct
  source 1..445: mol_type = other DNA
tgcctggttataattaacccagacatgtggctgccccccccccccccaacacctgctgcctctaaaaataaccctgtccctggtggatcgtcggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctggtacccgggagtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 69 - DNA, 415 bp, synthetic construct
  source 1..415: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatctgaggaaggtgggcaggcagcaggtgttgggggagttatttttagagcggggagttatttttagagcggaggaatggtggacacccaaatatggcgacggttcctcacggacacccaaatatggcgacgggccctggtacccgggagtgcccgacacccaaatatggcttgggaagggcagcaacattccttcggggcggtgtggggagagctcccgggactatataaaaacctgtgcaaggggacaggcggtcacacggacgtgaagcct

SEQ ID NO: 70 - DNA, 135 bp, synthetic construct
  source 1..135: mol_type = other DNA
tgaggaaggtgggcaggcagcaggtgttgggggagttatttttagagcggggagttatttttagagcggaggaatggtggacacccaaatatggcgacggttcctcacggacacccaaatatggcgacgggccct

SEQ ID NO: 71 - protein, 7 aa, synthetic construct
LGETTRP

SEQ ID NO: 72 - protein, 9 aa, synthetic construct
LALGETTRP

SEQ ID NO: 73 - protein, 1337 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPR

SEQ ID NO: 74 - protein, 1191 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 75 - protein, 1283 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLES

SEQ ID NO: 76 - protein, 1197 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 77 - protein, 1192 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 78 - protein, 1325 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 79 - protein, 1392 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEDTM

SEQ ID NO: 80 - protein, 1471 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM